(19) United States
(12) Patent Application Publication
(10) Pub. No.: US 2026/0347471 A1
(43) Pub. Date: Nov. 5, 2026

(54) ANTIBODIES BINDING TO CD3

(71) Applicant: Hoffmann-La Roche Inc., Little Falls, NJ (US)

(72) Inventors: Stephan MAERSCH, Paehl (DE); Ivan JELCIC, Riehen (CH); Christian Robert GASSNER, Penzberg (DE); Christian KLEIN, Bonstetten (CH); Johannes SAM, Baden (CH); Alain Charles TISSOT-DAGUETTE, Muenchen (DE); Alexander Christian BUJOTZEK, Muenchen (DE); Laurent LARIVIERE, Westendorf (DE)

(73) Assignee: Hoffmann-La Roche Inc., Little Falls, NJ (US)

(21) Appl. No.: 19/455,409

(22) Filed: Jan. 21, 2026

(30) Foreign Application Priority Data
Jan. 23, 2025 (EP) 25153473.1

(51) Int. Cl. C07K 16/28; A61K 39/00; A61P 37/02

(52) U.S. Cl. CPC C07K 16/2809; A61P 37/02; C07K 16/2887; A61K 2039/505; C07K 2317/24; C07K 2317/31; C07K 2317/522; C07K 2317/55; C07K 2317/565

(57) ABSTRACT

The present invention generally relates to antibodies that bind to CD3, including multispecific antibodies e.g. for activating T cells. In addition, the present invention relates to polynucleotides encoding such antibodies, and vectors and host cells comprising such polynucleotides. The invention further relates to methods for producing the antibodies, and to methods of using them in the treatment of disease.

### CROSS REFERENCE TO RELATED APPLICATIONS

This application claims priority under 35 U.S.C. 119(b) to European Patent Application No. 25153473.1, filed Jan. 23, 2025, which is incorporated herein by reference in its entirety.

### SEQUENCE LISTING

This application contains a Sequence Listing which has been submitted electronically in XML format and is hereby incorporated by reference in its entirety. Said XML copy, created on Jan. 14, 2026, is named P39832-US-1_Sequence_Listing.xml and is 44,576 bytes in size.

### FIELD OF THE INVENTION

The present invention generally relates to antibodies that bind to CD3, including multispecific antibodies e.g. for activating T cells. In addition, the present invention relates to polynucleotides encoding such antibodies, and vectors and host cells comprising such polynucleotides. The invention further relates to methods for producing the antibodies, and to methods of using them in the treatment of disease.

### BACKGROUND

CD3 (cluster of differentiation 3) is a protein complex composed of four subunits, the CD3γ chain, the CD3δ chain, and two CD3ε chains. CD3 associates with the T-cell receptor and the ζ chain to generate an activation signal in T lymphocytes.

CD3 has been extensively explored as drug target. Monoclonal antibodies targeting CD3 have been used as immunosuppressant therapies in autoimmune diseases such as type I diabetes, or in the treatment of transplant rejection. The anti-CD3 antibody muromonab-CD3 (OKT3) was the first monoclonal antibody ever approved for clinical use in humans, in 1985.

A more recent application of anti-CD3 antibodies is in the form of bispecific antibodies, binding CD3 on the one hand and a target cell antigen on the other hand. The simultaneous binding of such an antibody to both of its targets will force a temporary interaction between target cell and T cell, causing activation of any cytotoxic T cell and subsequent lysis of the target cell. Anti-CD3 antibodies, including bispecific such antibodies, are described e.g. in PCT publication no. WO 2021/255142 (incorporated herein by reference it its entirety).

An interesting target antigen for therapeutic purposes is for example CD20. Bispecific antibodies that bind to CD3 and CD20 are described e.g. in PCT publication no. WO 2016/020309 (incorporated herein by reference in its entirety). A particular bispecific antibody that binds to CD3 and CD20 is glofitamab (WHO Drug Information (International Nonproprietary Names for Pharmaceutical Substances), Recommended INN: List 83, 2020, vol. 34, no. 1, p. 39).

Efficacy and safety are key requirements of antibodies for therapeutic purposes. These can be hampered, however, by liabilities such as immunogenicity, non-specific binding, suboptimal pharmacokinetic (PK) properties and the like.

Given the tremendous therapeutic potential of antibodies, particularly bispecific antibodies for the activation of T cells, there is a need for CD3 antibodies with optimized properties.

### SUMMARY OF THE INVENTION

The present inventors aimed at generating antibodies that bind to CD3, with optimized properties for therapeutic use, e.g. as bispecific antibodies in cancer immunotherapy and other therapeutic fields.

High affinity binding to CD3 was not considered a priority. Rather to the contrary, the inventors aimed at modulating binding affinity to be in an intermediate to low range, in order to reduce the potential for adverse events such as excessive cytokine release induced by the antibodies (cytokine release syndrome (CRS) being a severe and common side effect of CD3 antibodies, in particular CD3 bispecific antibodies as used in cancer immunotherapy and other therapeutic fields). Minimization of adverse events is important in any therapeutic field, and in particular in non-oncology indications.

The inventors also aimed at minimizing immunogenicity of the antibodies, in order to minimize the risk of anti-drug antibodies (ADA) being formed against them, which can interfere with therapeutic efficacy or even pose a safety risk in patients.

Further, the inventors aimed at antibodies that bind to CD3-expressing cells with high specificity, to prevent undesired effects on non-target cells. The reduction of non-specific binding to cells and/or cellular accumulation (e.g. due to a reduction in positively charged patches and hydrophobic, surface-exposed residues) also serves to reduce non-specific clearance and thereby improve pharmacokinetic properties and may also contribute to reducing the risk of ADA formation.

At the same time, of course, the CD3 antibodies should show sufficient functional activity for therapeutic efficacy, and preserve desirable properties like produceability and stability.

The invention accordingly provides antibodies that bind to CD3 which combine the above-mentioned favorable properties for therapeutic use.

In one aspect, the invention provides an antibody that binds to CD3, wherein the antibody comprises a heavy chain variable region (VH_{CD3}) and a light chain variable region (VL_{CD3}), wherein
(a) the VH_{CD3} comprises the heavy chain complementarity determining region (HCDR) 1 of SEQ ID NO: 1, the HCDR 2 of SEQ ID NO: 9, and the HCDR 3 of SEQ ID NO: 10, and the VL_{CD3} comprises the light chain complementarity determining region (LCDR) 1 of SEQ ID NO: 5, the LCDR 2 of SEQ ID NO: 6, and the LCDR 3 of SEQ ID NO: 7;
(b) the VH_{CD3} comprises the heavy chain complementarity determining region (HCDR) 1 of SEQ ID NO: 1, the HCDR 2 of SEQ ID NO: 2, and the HCDR 3 of SEQ ID NO: 3, and the VL_{CD3} comprises the light chain complementarity determining region (LCDR) 1 of SEQ ID NO: 5, the LCDR 2 of SEQ ID NO: 6, and the LCDR 3 of SEQ ID NO: 7; or
(c) the VH_{CD3} comprises the heavy chain complementarity determining region (HCDR) 1 of SEQ ID NO: 1, the HCDR 2 of SEQ ID NO: 2, and the HCDR 3 of SEQ ID NO: 10, and the VL_{CD3} comprises the light chain complementarity determining region (LCDR) 1 of SEQ ID NO: 5, the LCDR 2 of SEQ ID NO: 6, and the LCDR 3 of SEQ ID NO: 13.

In one aspect,
(a) the VH_{CD3} comprises an amino acid sequence having at least 95%, at least 96%, at least 97%, at least 98%, at least 99% or 100% sequence identity to the VH sequence of SEQ ID NO: 11, and the VL_{CD3} comprises an amino acid sequence having at least 95%, at least 96%, at least 97%, at least 98%, at least 99% or 100% sequence identity to the VH sequence of SEQ ID NO: 8;
(b) the VH_{CD3} comprises an amino acid sequence having at least 95%, at least 96%, at least 97%, at least 98%, at least 99% or 100% sequence identity to the VH sequence of SEQ ID NO: 4, and the VL_{CD3} comprises an amino acid sequence having at least 95%, at least 96%, at least 97%, at least 98%, at least 99% or 100% sequence identity to the VH sequence of SEQ ID NO: 8; or
(c) the VH_{CD3} comprises an amino acid sequence having at least 95%, at least 96%, at least 97%, at least 98%, at least 99% or 100% sequence identity to the VH sequence of SEQ ID NO: 12, and the VL_{CD3} comprises an amino acid sequence having at least 95%, at least 96%, at least 97%, at least 98%, at least 99% or 100% sequence identity to the VH sequence of SEQ ID NO: 14.

In a further aspect, the invention provides an antibody that binds to CD3, comprising
(a) a heavy chain variable region (VH_{CD3}) comprising the amino acid sequence of SEQ ID NO: 11, and a light chain variable region (VL_{CD3}) comprising the amino acid sequence of SEQ ID NO: 8;
(b) a heavy chain variable region (VH_{CD3}) comprising the amino acid sequence of SEQ ID NO: 4, and a light chain variable region (VL_{CD3}) comprising the amino acid sequence of SEQ ID NO: 8; or
(c) a heavy chain variable region (VH_{CD3}) comprising the amino acid sequence of SEQ ID NO: 12, and a light chain variable region (VL_{CD3}) comprising the amino acid sequence of SEQ ID NO: 14.

In one aspect, the antibody is (a) a full-length antibody, particularly a full-length IgG antibody; or (b) an antibody fragment, particularly an antibody fragment selected from the group of an Fv molecule, a scFv molecule, a Fab molecule, and a F(ab′)₂ molecule.

In one aspect, the antibody is a multispecific antibody, particularly a bispecific antibody.

In one aspect, the antibody comprises (a) a first antigen binding domain that binds to CD3, comprising the VH_{CD3} and the VL_{CD3}, and (b) a second and optionally a third antigen binding domain that bind to a second antigen. In one aspect, the first antigen binding domain, the second antigen binding domain and/or the third antigen binding domain is a Fab molecule. In one aspect, the first antigen binding domain is a Fab molecule wherein the variable domains VH and VL or the constant domains CH1 and CL of the Fab heavy and light chains are replaced by each other. In one aspect, the second antigen binding domain and, where present, the third antigen binding domain are each a conventional Fab molecule. In one aspect, in the constant domain CL of the second antigen binding domain and, where present, the third antigen binding domain the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the constant domain CH1 of the second antigen binding domain and, where present, the third antigen binding domain the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In one aspect, the second antigen is a target cell antigen, particularly a B-cell antigen. In one aspect, the second antigen is CD20. In one aspect, the second antigen binding domain and, where present, the third antigen binding domain comprises a heavy chain variable region (VH_{CD20}) comprising the heavy chain complementarity determining region (HCDR) 1 of SEQ ID NO: 15, the HCDR 2 of SEQ ID NO: 16, and the HCDR 3 of SEQ ID NO: 17, and a light chain variable region (VL_{CD20}) comprising the light chain complementarity determining region (LCDR) 1 of SEQ ID NO: 19, the LCDR 2 of SEQ ID NO: 20, and the LCDR 3 of SEQ ID NO: 21. In one aspect, the second antigen binding domain and, where present, the third antigen binding domain comprises a VH_{CD20} comprising an amino acid sequence having at least 95%, at least 96%, at least 97%, at least 98%, at least 99% or 100% sequence identity to the VH sequence of SEQ ID NO: 18, and a VL_{CD20} comprising an amino acid sequence having at least 95%, at least 96%, at least 97%, at least 98%, at least 99% or 100% sequence identity to the VH sequence of SEQ ID NO: 22.

In one aspect, the first antigen binding domain, the second antigen binding domain and, where present, the third antigen binding domain are each a Fab molecule and (i) the first antigen binding domain is fused at the C-terminus of the Fab heavy chain to the N-terminus of the Fab heavy chain of the second antigen binding domain, or (ii) the second antigen binding domain is fused at the C-terminus of the Fab heavy chain to the N-terminus of the Fab heavy chain of the first antigen binding domain.

In one aspect, (a) the antibody comprises an Fc domain composed of a first and a second subunit, (b) (i) the first antigen binding domain is fused at the C-terminus of the Fab heavy chain to the N-terminus of one of the subunits of the Fc domain, or (ii) the second antigen binding domain is fused at the C-terminus of the Fab heavy chain to the N-terminus of one of the subunits of the Fc domain, and (c), where present, the third antigen binding domain is fused at the C-terminus of the Fab heavy chain to the N-terminus of the other one of the subunits of the Fc domain.

In one aspect, the antibody comprises an Fc domain composed of a first and a second subunit. In one aspect, the Fc domain is an IgG Fc domain, particularly an IgG₁ Fc domain, more particularly a human IgG₁ Fc domain. In one aspect, the Fc domain comprises a modification, particularly an amino acid substitution or combination of amino acid substitutions, promoting the association of the first and the second subunit of the Fc domain. In one aspect, the Fc domain comprises a modification, particularly an amino acid substitution or combination of amino acid substitutions, that reduces the binding affinity to an Fc receptor and/or effector function of the antibody, as compared to a corresponding antibody comprising an Fc domain without such modification. In one aspect, the Fc domain comprises a modification, particularly an amino acid substitution or combination of amino acid substitutions, that increases the binding affinity to an FcRn receptor and/or the serum half-life of the antibody, as compared to a corresponding antibody comprising an Fc domain without such modification.

In one aspect, the antibody comprises
(a) a first heavy chain (HC1) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 23, a first light chain (LC1) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 28, a second heavy chain (HC2) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 25, and a second light chain (LC2) (particularly two LC2) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 26;
(b) a first heavy chain (HC1) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 23, a first light chain (LC1) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 27, a second heavy chain (HC2) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 25, and a second light chain (LC2) (particularly two LC2) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 26; or
(c) a first heavy chain (HC1) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 24, a first light chain (LC1) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 29, a second heavy chain (HC2) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 25, and a second light chain (LC2) (particularly two LC2) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 26.

According to a further aspect of the invention there is provided an isolated polynucleotide encoding an antibody of the invention, and a host cell comprising the isolated polynucleotide of the invention.

In another aspect is provided a method of producing an antibody that binds to CD3, comprising the steps of (a) culturing the host cell of the invention under conditions suitable for the expression of the antibody and optionally (b) recovering the antibody. The invention also encompasses an antibody that binds to CD3 produced by the method of the invention.

The invention further provides a pharmaceutical composition comprising the antibody of the invention and a pharmaceutically acceptable carrier.

Also encompassed by the invention are methods of using the antibody and pharmaceutical composition of the invention. In one aspect the invention provides an antibody or pharmaceutical composition according to the invention for use as a medicament. In one aspect is provided an antibody or pharmaceutical composition according to the invention for use in the treatment of a disease.

Also provided is the use of an antibody or pharmaceutical composition according to the invention in the manufacture of a medicament, the use of an antibody or pharmaceutical composition according to the invention in the manufacture of a medicament for the treatment of a disease.

The invention also provides a method of treating a disease in an individual, comprising administering to said individual an effective amount of the antibody or pharmaceutical composition according to the invention.

According to any of the above aspects, in a specific aspect, the disease is cancer or an autoimmune disease.

### BRIEF DESCRIPTION OF THE DRAWINGS

FIG. 1. Exemplary configurations of the (multispecific) antibodies of the invention, comprising a “crossover” Fab molecule that binds to CD3 (first antigen binding domain), one or two conventional Fab molecule that binds to a second antigen (second and third antigen binding domain), and an Fc domain. (FIG. 1A) “1+1” molecule with the second antigen binding domain fused at its C-terminus to the N-terminus of the first antigen binding domain, and the first antigen binding domain fused at its C-terminus to the N-terminus of the Fc domain. (FIG. 1B) “1+1” molecule with the first and second antigen binding domains each fused at its C-terminus to the N-terminus of the Fc domain. (FIG. 1C) “1+1” molecule with the first antigen binding domain fused at its C-terminus to the N-terminus of the second antigen binding domain, and the second antigen binding domain fused at its C-terminus to the N-terminus of the Fc domain. (FIG. 1D) “2+1” molecule with the second antigen binding domain fused at its C-terminus to the N-terminus of the first antigen binding domain, and the first antigen binding domain and the third antigen binding domain each fused at its C-terminus to the N-terminus of the Fc domain. (FIG. 1E) “2+1” molecule with the first antigen binding domain fused at its C-terminus to the N-terminus of the second antigen binding domain, and the second antigen binding domain and the third antigen binding domain each fused at its C-terminus to the N-terminus of the Fc domain. Black dot: optional modification promoting heterodimerization (e.g. “knob-into-hole”) in the Fc domain. ++, −−: optional charge modifications (e.g. CH1(EE)=147E, 213E; CL(RK)=123R, 124K) in the CH1 and CL domains of the second and third antigen binding domains. The first antigen binding domain is depicted as comprising an exchange of VH and VL regions, but may alternatively comprise an exchange of the CH1 and CL domains (in such aspects without charge modifications introduced in CH1 and CL domains of the second and third antigen binding domains).

FIG. 2. (FIG. 2A) Schematic illustration of the “2+1” T-cell bispecific antibody (TCB) molecules used in the Examples. (FIG. 2B-2E) Components for the assembly of the TCB: light chain of anti-target antigen Fab molecule with charge modifications in CL (FIG. 2B), light chain of anti-CD3 crossover Fab molecule (FIG. 2C), heavy chain with charge modifications in CH1 and knob and PG LALA mutations in Fc region (FIG. 2D), heavy chain with hole and PG LALA mutations in Fc region (FIG. 2E). EE=147E, 213E; RK=123R, 124K.

FIG. 3. Result of the ELISA of Example 7, assessing non-specific binding of different anti-CD3 antibodies to CD3-negative cells. Signals are given as % of the signal for glofitamab, which was used as reference in the assay.

FIG. 4. LUCA rate (cellular accumulation rate) for the optimized anti-CD3 antibodies (as CD3×CD20 TCBs P1AN0288 and P1AN0289) as compared to glofitamab, wherein glofitamab is normalized to a LUCA rate of 1.

FIG. 5. B-cell depletion induced by a set of anti-CD3 antibodies (as CD3×CD20 TCBs) of different CD3 affinities, as determined in the assay of Example 9.

FIG. 6. IL-6 secretion induced by a set of anti-CD3 antibodies (as CD3×CD20 TCBs) of different CD3 binding affinities, as determined in the assay of Example 9. Eₘₐₓ was not calculable for P1AN0290.

FIG. 7. Schematic overview of the human tonsil assay according to Example 10.

FIG. 8. B-cell depletion in cultured tonsillar mononuclear cells (tMNCs) with the various compounds and concentrations upon 72 h, according to Example 10. Depicted are the overall counts of B-cells (FIG. 8A), and memory B-cell subsets (switched memory as defined by CD27+IgD-(FIG. 8B) and unswitched memory as defined by CD27+IgD+(FIG. 8C)). Exemplary data from one donor.

FIG. 9. Cytokine release in cell culture supernatants of compound-treated tonsillar mononuclear cells (tMNCs), according to Example 10. Depicted are IL-2 (FIG. 9A), IFN-γ (FIG. 9B) and TNF (FIG. 9C). Exemplary data from one donor.

FIG. 10. Design of the in vivo study in Example 11.

FIG. 11. Body weight kinetics as determined in the in vivo study of Example 11. Depicted are (FIG. 11A) glofitamab, (FIG. 11B) P1AM3067, (FIG. 11C) P1AN0288, (FIG. 11D) P1AN0289. Black circles=vehicle, open circles=0.5 mg/kg, black squares=0.15 mg/kg, open squares=0.05 mg/kg.

FIG. 12. Serum cytokine levels as determined in the in vivo study of Example 11. Depicted are (FIG. 12A) IL-6, (FIG. 12B) IFN-γ, (FIG. 12C) TNF-α. Study groups (A-M) are indicated according to the labelling in FIG. 10 and Table 12.

FIG. 13. B-cell depletion as determined in the in vivo study of Example 11. Depicted are (FIG. 13A) spleen, (FIG. 13B) blood. Study groups (A-M) are indicated according to the labelling in FIG. 10 and Table 12.

### DETAILED DESCRIPTION OF THE INVENTION

### I. Definitions

Terms are used herein as generally used in the art, unless otherwise defined in the following.

As used herein, the terms “first”, “second” or “third” with respect to antigen binding domains etc., are used for convenience of distinguishing when there is more than one of each type of moiety.

Use of these terms is not intended to confer a specific order or orientation of the moiety unless explicitly so stated.

The terms “anti-CD3 antibody” and “an antibody that binds to CD3” refer to an antibody that is capable of binding CD3 with sufficient affinity such that the antibody is useful as a diagnostic and/or therapeutic agent in targeting CD3. In one aspect, the extent of binding of an anti-CD3 antibody to an unrelated, non-CD3 protein is less than about 10% of the binding of the antibody to CD3 as measured, e.g., by surface plasmon resonance (SPR). In certain aspects, an antibody that binds to CD3 has a dissociation constant (K_{D}) of 1 μM, ≤500 nM, ≤200 nM, or 100 nM, particularly a K_{D} of 500 nM, as measured by SPR at 25 °C. In a particular aspect, an antibody that binds to CD3 has a K_{D} of about 100-500 nM, particularly about 150-400 nM, as measured by SPR at 25 °C.

By “specific binding” is meant that the binding is selective for the antigen and can be discriminated from unwanted or non-specific interactions. Suitable assays for determining the specificity of an antibody of the present invention are described herein, including in the Examples hereinbelow. In some aspects, the extent of binding of an antibody to an unrelated protein is less than about 10% of the binding of the antibody to the antigen as measured, e.g., by SPR.

The term “antibody” encompasses various antibody structures exhibiting the desired antigen-binding activity, including but not limited to monoclonal antibodies, polyclonal antibodies, multispecific antibodies (e.g. bispecific antibodies), and antibody fragments.

An “antibody fragment” refers to a molecule other than a full-length antibody that comprises a portion of a full-length antibody that binds the antigen to which the full-length antibody binds. Examples of antibody fragments include but are not limited to Fv molecules, Fab molecules, Fab′ molecules, Fab′-SH molecules, F(ab′)₂ molecules, diabodies, linear antibodies, single-chain antibody molecules (e.g. scFv and scFab molecules), single-domain antibody molecules, and multispecific (e.g. bispecific) antibodies formed from antibody fragments. For a review of certain antibody fragments, see Hollinger and Hudson, Nature Biotechnology 23:1126-1136 (2005).

The term “full-length antibody” refers to an antibody having the structure of an immunoglobulin molecule comprising two light chains and two heavy chains, and comprising an Fc domain as defined herein. In one aspect, the antibody is a full-length IgG₁ antibody.

The term “monoclonal antibody” as used herein refers to an antibody obtained from a population of substantially homogeneous antibodies, i.e. the antibodies forming this population are essentially identical, except for possible post-translational modifications arising e.g. during manufacturing and/or storage. These antibodies are directed against the same epitope (or the same group of epitopes in the case of multispecific monoclonal antibodies, e.g. the same pair of epitopes in the case of bispecific monoclonal antibodies). This definition expressly excludes polyclonal antibody preparations which are mixtures of antibodies directed against different epitopes. Monoclonal antibodies in accordance with the present invention may be made by a variety of techniques, including but not limited to hybridoma methodology, recombinant DNA methods, phage-display methods, and methods utilizing transgenic animals containing all or part of the human immunoglobulin loci, such methods and other exemplary methods for making monoclonal antibodies being described herein.

An “isolated” antibody is one which has been separated from a component of its natural environment. In one aspect, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC, affinity chromatography, size exclusion chromatography) methods. For review of methods for assessment of antibody purity, see, e.g., Flatman et al., J. Chromatogr. B 848:79-87 (2007). In one aspect, the antibody provided by the present invention is an isolated antibody.

A “humanized” antibody refers to an antibody comprising amino acid residues from non-human CDRs and amino acid residues from human FRs. In one aspect, a humanized antibody will comprise substantially all of at least one, and typically two, variable domains, in which all or substantially all of the CDRs correspond to those of a non-human antibody, and all or substantially all of the FRs correspond to those of a human antibody. One or more FR residues in a humanized antibody may be substituted with corresponding residues from a non-human antibody (e.g., the antibody from which the CDR residues are derived), e.g., to restore or improve antibody specificity or affinity. Such variable domains are referred to herein as “humanized variable region”. A humanized antibody optionally may comprise at least a portion of an antibody constant region derived from a human antibody. A “humanized form” of an antibody, e.g., a non-human antibody, refers to an antibody that has undergone humanization.

Typically, a non-human antibody is humanized to reduce immunogenicity to humans, while retaining the specificity and affinity of the parental non-human antibody.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. In one aspect, a human antibody is derived from a non-human transgenic mammal, for example a mouse, a rat, or a rabbit. In one aspect, a human antibody is derived from a hybridoma cell line. Antibodies or antibody fragments isolated from human antibody libraries are also considered human antibodies or human antibody fragments herein.

The term “antigen binding domain” refers to the part of an antibody that comprises the area which binds to and is complementary to part or all of an antigen. An antigen binding domain may be provided by, for example, one or more antibody variable domains (also called antibody variable regions). In a preferred aspect, an antigen binding domain comprises an antibody light chain variable domain (VL) and an antibody heavy chain variable domain (VH).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and complementarity determining regions (CDRs). See, e.g., Kindt et al., Kuby Immunology, 6^{th} ed., W.H. Freeman & Co., page 91 (2007). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

Glutamine or glutamate residues at the N-terminus of antibody heavy or light chains may be converted to pyro-glutamate spontaneously (see e.g. Liu et al., Journal of Pharmaceutical Sciences 97, 2426-2447 (2008), Rehder et al., Journal of Chromatography A 1102, 164-175 (2006), Chelius et al., Anal Chem 78, 2370-2376 (2006)). Hence, variable regions or variable domains disclosed herein which comprise either a glutamine (Q) or a glutamate (E) amino acid residue at the N-terminus of an antibody heavy or light chain, may comprise an N-terminal pyro-glutamate (pyroE) residue instead of the N-terminal Q or E residue. Likewise, antibody heavy chains or light chains disclosed herein which comprise either a glutamine (Q) or a glutamate (E) amino acid residue at the N-terminus, may comprise an N terminal pyro-glutamate (pyroE) residue instead of the N-terminal Q or E residue. Accordingly, for each antibody heavy chain, light chain, or variable domain or region sequence disclosed herein that contains an N-terminal Q or E residue, the corresponding sequence with an N-terminal pyroE residue is also encompassed.

Unless otherwise indicated, CDR residues and other residues in the variable domain (e.g., FR residues) are numbered herein according to Kabat et al., Sequences of Proteins of Immunological Interest, 5th Ed. Public Health Service, National Institutes of Health, Bethesda, MD (1991).

The term “complementarity determining region” or “CDR” as used herein refers to each of the regions of an antibody variable domain which are hypervariable in sequence and which determine antigen binding specificity. Generally, antibodies comprise six CDRs: three in the VH (HCDR1, HCDR2, HCDR3), and three in the VL (LCDR1, LCDR2, LCDR3). CDRs are defined by a variety of methods/systems by those skilled in the art. These systems and/or definitions have been developed and refined over a number of years and include Kabat, Chothia, IMGT, AbM, and Contact. The Kabat definition is based on sequence variability and generally is the most commonly used. The Chothia definition is based on the location of the structural loop regions. The IMGT system is based on sequence variability and location within the structure of the variable domain. The AbM definition is a compromise between Kabat and Chothia. The Contact definition is based on analyses of the available antibody crystal structures. Software programs (e.g., abYsis: http://www.abysis.org/abysis/sequence_input/key_annotation/key_annotation.cgi) are available and known to those of skill in the art for analysis of antibody sequences and determination of CDRs.

Exemplary CDRs herein include (numbering of amino acid residues according to the reference cited, i.e. Chothia numbering for the Chothia and Contact definition, Kabat numbering for the Kabat definition and IMGT numbering for the IMGT definition):
(a) hypervariable loops occurring at amino acid residues 26-32 (L1), 50-52 (L2), 91-96 (L3), 26-32 (H1), 53-55 (H2), and 96-101 (H3), according to Chothia and Lesk, J. Mol. Biol. 196:901-917 (1987) (“Chothia definition”);
(b) CDRs occurring at amino acid residues 24-34 (L1), 50-56 (L2), 89-97 (L3), 31-35bB (H1), 50-65 (H2), and 95-102 (H3), according to Kabat et al., Sequences of Proteins of Immunological Interest, 5th Ed. Public Health Service, National Institutes of Health, Bethesda, MD (1991) (“Kabat definition”);
(c) antigen contacts occurring at amino acid residues 3027c-36 (L1), 46-55 (L2), 89-96 (L3), 30-35b (H1), 47-58 (H2), and 93-101 (H3), according to MacCallum et al. J. Mol. Biol. 262: 732-745 (1996) (“Contact definition”); and
(d) CDRs occurring at amino acid residues residues 27-38 (L1), 56-65 (L2), 105-117 (L3), 27-38 (H1), 56-65 (H2), and 105-117 (H3), according to Lefranc et al. Dev. Comp. Immunol. 27: 55-77 (2003) (“IMGT definition”).

Unless otherwise indicated, the CDRs are determined herein according to Kabat et al., supra. One of skill in the art will understand that the CDR designations can also be determined according to Chothia, supra, MacCallum, supra, Lefranc, supra, or any other scientifically accepted definition/system.

“Framework” or “FR” refers to variable domain residues other than complementarity determining regions (CDRs). The FR of a variable domain generally consists of four FR domains: FR1, FR2, FR3, and FR4. Accordingly, the CDR and FR sequences generally appear in the following order in VH (or VL): FR1-HCDR1(LCDR1)-FR2-HCDR2(LCDR2)-FR3-HCDR3(LCDR3)-FR4.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some aspects, the number of amino acid changes is 10 or less, 9 or less, 8 or less, 7 or less, 6 or less, 5 or less, 4 or less, 3 or less, or 2 or less. In some aspects, the VL acceptor human framework is identical in sequence to the VL human immunoglobulin framework sequence or human consensus framework sequence.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3.

The terms “constant region derived from human origin” or “human constant region” as used herein denotes a constant region of a human antibody, in particular a heavy chain constant region of a human antibody of the subclass IgG₁, IgG₂, IgG₃, or IgG₄ and/or a light chain kappa or lambda constant region. Such constant regions are well known in the state of the art and e.g. described by Kabat, E. A., et al., Sequences of Proteins of Immunological Interest, 5th ed., Public Health Service, National Institutes of Health, Bethesda, MD (1991). Unless otherwise specified herein, numbering of amino acid residues in the constant region is according to the numbering system as described in Kabat, E. A. et al., Sequences of Proteins of Immunological Interest, 5th ed., Public Health Service, National Institutes of Health, Bethesda, MD (1991), NIH Publication 91-3242. Specifically, the Kabat numbering system (referred to as “numbering according to Kabat” or “Kabat numbering” herein; see pages 647-660 of Kabat et al., supra) is used for the light chain constant domain of kappa and lambda isotype, and the Kabat EU index numbering system (referred to as “numbering according to Kabat EU index”, “Kabat EU index numbering” or “Kabat EU numbering” herein, see pages 661-723 of Kabat et al., supra) is used for the heavy chain constant domains.

The term “immunoglobulin molecule” herein refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable domain (VH), also called a variable heavy domain or a heavy chain variable region, followed by three constant domains (CH1, CH2, and CH3), also called a heavy chain constant region. Similarly, from N- to C-terminus, each light chain has a variable domain (VL), also called a variable light domain or a light chain variable region, followed by a constant light (CL) domain, also called a light chain constant region. The heavy chain of an immunoglobulin may be assigned to one of five types (or classes), called α (IgA), δ (IgD), ε (IgE), γ (IgG), or μ (IgM), some of which may be further divided into subtypes (or subclasses), e.g. γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (λ), based on the amino acid sequence of its constant domain.

An immunoglobulin essentially consists of two Fab molecules and an Fc domain, linked via the immunoglobulin hinge region.

The “class” of an antibody or immunoglobulin refers to the type of constant domain or constant region possessed by its heavy chain. There are five major classes of antibodies: IgA, IgD, IgE, IgG, and IgM. The heavy chain constant domains that correspond to the different classes of immunoglobulins are called α, δ, ε, γ, and μ, respectively. Several of the antibody classes may be further divided into subclasses (isotypes), e.g., IgG₁, IgG₂, IgG₃, IgG₄, IgA₁, and IgA₂, with corresponding heavy chain constant domains γ₁ (IgG₁), γ₂ (IgG₂), γ₃ (IgG₃), γ₄ (IgG₄), α₁ (IgA₁) and α₂ (IgA₂). The light chain of an antibody may be assigned to one of two types, called kappa (κ) and lambda (λ), based on the amino acid sequence of its constant domain.

A “Fab molecule” or “Fab fragment” refers to a protein consisting of the VH and CH1 domain of the heavy chain (the “Fab heavy chain”) and the VL and CL domain of the light chain (the “Fab light chain”) of an immunoglobulin.

By a “crossover” Fab molecule (also termed “Crossfab”) is meant a Fab molecule wherein the variable domains or the constant domains of the Fab heavy and light chain are exchanged (i.e. replaced by each other), i.e. the crossover Fab molecule comprises a peptide chain composed of the light chain variable domain VL and the heavy chain constant domain 1 CH1 (VL-CH1, in N- to C-terminal direction), and a peptide chain composed of the heavy chain variable domain VH and the light chain constant domain CL (VH-CL, in N- to C-terminal direction). For clarity, in a crossover Fab molecule wherein the variable domains of the Fab light chain and the Fab heavy chain are exchanged, the peptide chain comprising the heavy chain constant domain 1 (CH1) is referred to herein as the “heavy chain” of the (crossover) Fab molecule. Conversely, in a crossover Fab molecule wherein the constant domains of the Fab light chain and the Fab heavy chain are exchanged, the peptide chain comprising the heavy chain variable domain (VH) is referred to herein as the “heavy chain” of the (crossover) Fab molecule.

In contrast thereto, by a “conventional” Fab molecule is meant a Fab molecule in its natural format, i.e. comprising a heavy chain composed of the heavy chain variable and constant domains (VH-CH1, in N- to C-terminal direction), and a light chain composed of the light chain variable and constant domains (VL-CL, in N- to C-terminal direction).

The term “Fc domain” or “Fc region” (used interchangeably) herein is used to define a C-terminal region of an immunoglobulin heavy chain that contains at least a portion of the constant region. The term includes native sequence Fc domains and variant Fc domains. In one aspect, a human IgG heavy chain Fc domain extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case in particular where the final two C-terminal amino acids of the heavy chain are glycine (G446) and lysine (K447, numbering according to Kabat EU index). Therefore, the C-terminal lysine (Lys447), or the C-terminal glycine (Gly446) and lysine (Lys447), of the Fc domain may or may not be present. Amino acid sequences of heavy chains including an Fc domain (or a subunit of an Fc domain as defined herein) are denoted herein without C-terminal lysine if not indicated otherwise. The corresponding sequence including a C-terminal lysine residue is also encompassed, however. Accordingly, in one aspect, a heavy chain including an Fc domain (subunit) as specified herein comprises an additional C-terminal lysine residue (K447, Kabat EU numbering). Also encompassed is the corresponding sequence without the C-terminal glycine residue. Accordingly, in one aspect, a heavy chain including an Fc domain (subunit) as specified herein lacks the C-terminal glycine residue (G446, Kabat EU numbering). In such a heavy chain, the C-terminal amino acid residue may be proline (P445, Kabat EU numbering) or proline amide (P445-NH₂, Kabat EU numbering). Unless otherwise specified herein, numbering of amino acid residues in the Fc domain or heavy chain constant region is according to the EU numbering system, also called the EU index, as described in Kabat et al., Sequences of Proteins of Immunological Interest, 5th Ed. Public Health Service, National Institutes of Health, Bethesda, MD, 1991 (see also above). A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

By “fused” is meant that the components (e.g. a Fab molecule and an Fc domain subunit) are linked by peptide bonds, either directly or via one or more peptide linkers.

The term “multispecific” means that the antibody is able to specifically bind to at least two distinct antigenic determinants. A multispecific antibody can be, for example, a bispecific antibody.

Typically, a bispecific antibody comprises two antigen binding sites, each of which is specific for a different antigenic determinant. In certain aspects, the multispecific (e.g. bispecific) antibody is capable of simultaneously binding two antigenic determinants, particularly two antigenic determinants expressed on two distinct cells.

The term “valent” as used herein denotes the presence of a specified number of antigen binding sites in an antibody. As such, e.g. the term “monovalent” denotes the presence of one (and not more than one) antigen binding site specific for the antigen in the antibody.

An “antigen binding site” refers to the site, i.e. one or more amino acid residues, of an antigen binding molecule which provides interaction with the antigen. For example, the antigen binding site of an antibody comprises amino acid residues from the complementarity determining regions (CDRs). A native immunoglobulin molecule typically has two antigen binding sites, a Fab molecule typically has a single antigen binding site.

As used herein, the term “antigenic determinant” or “antigen” refers to a site (e.g. a contiguous stretch of amino acids or a conformational configuration made up of different regions of non-contiguous amino acids) on a polypeptide macromolecule to which an antigen binding domain binds, forming an antigen binding domain-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). In a preferred aspect, the antigen is a human protein.

“CD3” as used herein refers to human CD3. “CD3E” refers to the epsilon subunit of human CD3. The amino acid sequence of human CD3E is shown in SEQ ID NO: 30 (without signal peptide). See also UniProt (www.uniprot.org) accession no. P07766 (entry version 233), or NCBI (www.ncbi.nlm.nih.gov/) RefSeq NP_000724.1. In certain aspects the antibody of the invention binds to an epitope of CD3, particularly of CD3E, that is conserved among the CD3 antigens from different species, particularly human and cynomolgus (Macaca fascicularis) CD3. The amino acid sequence of cynomolgus CD3E is shown in SEQ ID NO: 31 (without signal peptide). See also NCBI GenBank no. BAB71849.1.

A “target cell antigen” as used herein refers to an antigenic determinant presented on the surface of a target cell, for example a cell in a tumor such as a cancer cell or a cell of the tumor stroma (in that case a “tumor cell antigen”). Preferably, the target cell antigen is not CD3, and/or is expressed on a different cell than CD3. In one aspect, the target cell antigen is a B-cell antigen (i.e. an antigenic determinant presented on the surface of a B-cell). In one aspect, the target cell antigen is CD20, specifically human CD20.

“CD20”, also known as “B-lymphocyte antigen B1”, as used herein refers to human CD20. See for the human protein UniProt (www.uniprot.org) accession no. P11836 (entry version 221), or NCBI (www.ncbi.nlm.nih.gov/) RefSeq NP_068769.2. An exemplary sequence of human CD20 is given in SEQ ID NO: 32.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., an antibody and an antigen). The affinity of a molecule X for its partner Y can generally be represented by the dissociation constant (K_{D}). Affinity can be measured by well-established methods known in the art, including those described herein. A preferred method for measuring affinity is Surface Plasmon Resonance (SPR).

“Reduced binding”, for example reduced binding to an Fc receptor, refers to a decrease in affinity for the respective interaction, as measured for example by SPR. For clarity, the term includes also reduction of the affinity to zero (or below the detection limit of the analytic method), i.e. complete abolishment of the interaction. Conversely, “increased binding” refers to an increase in binding affinity for the respective interaction.

“T cell activation” as used herein refers to one or more cellular response of a T lymphocyte, particularly a cytotoxic T lymphocyte, selected from: proliferation, differentiation, cytokine secretion, cytotoxic effector molecule release, cytotoxic activity, and expression of activation markers. Suitable assays to measure T cell activation are known in the art and described herein.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein preferably includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which may be non-identical in the sense that further components fused to each of the subunits (e.g. antigen binding domains) are not the same. In some aspects, the modification promoting the association of the first and the second subunit of the Fc domain comprises an amino acid mutation in the Fc domain, specifically an amino acid substitution. In a preferred aspect, the modification promoting the association of the first and the second subunit of the Fc domain comprises a separate amino acid mutation, specifically an amino acid substitution, in each of the two subunits of the Fc domain.

The term “effector functions” refers to those biological activities attributable to the Fc domain of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

An “activating Fc receptor” is an Fc receptor that following engagement by an Fc domain of an antibody elicits signaling events that stimulate the receptor-bearing cell to perform effector functions. Human activating Fc receptors include FcγRIIIa (CD16a), FcγRI (CD64), FcγRIIa (CD32), and FcαRI (CD89).

Antibody-dependent cell-mediated cytotoxicity (ADCC) is an immune mechanism leading to the lysis of antibody-coated target cells by immune effector cells. The target cells are cells to which antibodies or derivatives thereof comprising an Fc domain specifically bind, generally via the protein part that is N-terminal to the Fc domain. As used herein, the term “reduced ADCC” is defined as either a reduction in the number of target cells that are lysed in a given time, at a given concentration of antibody in the medium surrounding the target cells, by the mechanism of ADCC defined above, and/or an increase in the concentration of antibody in the medium surrounding the target cells, required to achieve the lysis of a given number of target cells in a given time, by the mechanism of ADCC. The reduction in ADCC is relative to the ADCC mediated by the same antibody produced by the same type of host cells, using the same standard production, purification, formulation and storage methods (which are known to those skilled in the art), but that has not been engineered. For example, the reduction in ADCC mediated by an antibody comprising in its Fc domain an amino acid substitution that reduces ADCC, is relative to the ADCC mediated by the same antibody without this amino acid substitution in the Fc domain. Suitable assays to measure ADCC are well known in the art (see e.g. PCT publication no. WO 2006/082515 or PCT publication no. WO 2012/130831).

As used herein, the terms “engineer, engineered, engineering”, are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches.

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics, e.g., reduced binding to an Fc receptor, or increased association with another peptide. Amino acid sequence deletions and insertions include amino- and/or carboxy-terminal deletions and insertions of amino acids. Preferred amino acid mutations are amino acid substitutions. For the purpose of altering e.g. the binding characteristics of an Fc domain, non-conservative amino acid substitutions, i.e. replacing one amino acid with another amino acid having different structural and/or chemical properties, are particularly preferred. Amino acid substitutions include replacement by non-naturally occurring amino acids or by naturally occurring amino acid derivatives of the twenty standard amino acids (e.g. 4-hydroxyproline, 3-methylhistidine, ornithine, homoserine, 5-hydroxylysine). Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful. Various designations may be used herein to indicate the same amino acid mutation. For example, a substitution from proline at position 329 of the Fc domain to glycine can be indicated as 329G, G329, G329, P329G, or Pro329Gly.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, % amino acid sequence identity values are generated using the ggsearch program of the FASTA package version 36.3.8c or later with a BLOSUM50 comparison matrix. The FASTA program package was authored by W. R. Pearson and D. J. Lipman (“Improved Tools for Biological Sequence Analysis”, PNAS 85 (1988) 2444-2448), W. R. Pearson (“Effective protein sequence comparison” Meth. Enzymol. 266 (1996) 227-258), and Pearson et. al. (Genomics 46 (1997) 24-36) and is publicly available from www.fasta.bioch.virginia.edu/fasta_www2/fasta_down.shtmi or www.ebi.ac.uk/Tools/sss/fasta. Alternatively, a public server accessible at fasta.bioch.virginia.edu/fasta_www2/index.cgi can be used to compare the sequences, using the ggsearch (global protein:protein) program and default options (BLOSUM50; open: -10; ext: -2; Ktup=2) to ensure a global, rather than local, alignment is performed. Percent amino acid identity is given in the output alignment header.

The term “polynucleotide” or “nucleic acid molecule” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler et al. (2017) Nature Medicine 23:815-817, or EP 2101823 B1).

An “isolated” nucleic acid molecule refers to a nucleic acid molecule that has been separated from a component of its natural environment. An isolated nucleic acid molecule includes a nucleic acid molecule contained in cells that ordinarily contain the nucleic acid molecule, but the nucleic acid molecule is present extrachromosomally or at a chromosomal location that is different from its natural chromosomal location.

“Isolated polynucleotide (or nucleic acid) encoding an antibody” refers to one or more polynucleotide molecules encoding antibody heavy and light chains (or fragments thereof), including such polynucleotide molecule(s) in a single vector or separate vectors, and such polynucleotide molecule(s) present at one or more locations in a host cell.

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. Suitable host cells may include, for example, CHO cells, HEK-293 cells, Expi293F cells, PER.C6 cells, NSO cells, lymphocytic cells, prokaryotic cells such as E. coli, and other eukaryotic hosts such as plant cells and fungi. Human host cells are included with the proviso that they are not used within the human body. In one aspect, the host cell of the invention is a eukaryotic cell, particularly a mammalian cell. In one aspect, the host cell is not a cell within a human body.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the composition would be administered. Specifically, the term refers to a preparation of the antibody of the invention and one or more pharmaceutically acceptable carriers or excipients.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, surfactant and/or preservative.

As used herein, “treatment” (and grammatical variations thereof such as “treat” or “treating”) refers to clinical intervention in an attempt to alter the natural course of a disease in the individual being treated, performed during the course of clinical pathology. Desirable effects of treatment include, but are not limited to, alleviation of symptoms, diminishment of any direct or indirect pathological consequences of the disease, decreasing the rate of disease progression, amelioration or palliation of the disease state, and remission or improved prognosis. For example, an individual is successfully “treated” if one or more symptoms associated with the disease are mitigated or eliminated, the individual's quality of life is improved, the dose of other medications required to treat the disease can be reduced, and/or the individual's survival is prolonged.

An “effective amount” of an agent, e.g., a pharmaceutical composition, refers to an amount of the antibody or medicament effective, at dosages and for periods of time necessary, to achieve the desired treatment as defined above.

An “individual” or “subject” is a mammal. In one aspect, the individual or subject is a human. In one aspect, the individual is in need of treatment.

The term “package insert” is used to refer to instructions customarily included in commercial packages of therapeutic products, that contain information about the indications, usage, dosage, administration, combination therapy, contraindications and/or warnings concerning the use of such therapeutic products.

### II. Compositions and Methods

The invention provides antibodies that bind CD3, including multispecific antibodies that bind CD3 and a second antigen. The antibodies show reduced immunogenicity risk, non-specific binding to cells and cellular accumulation, combined with other favorable properties for therapeutic application, e.g. with respect to efficacy and safety, pharmacokinetics, as well as produceability. Antibodies of the invention are useful, e.g., for the treatment of diseases such as cancer.

### A. Anti-CD3 Antibodies

In one aspect, the invention provides antibodies that bind to CD3. In one aspect, provided are isolated antibodies that bind to CD3. In one aspect, the invention provides antibodies that specifically bind to CD3. In one aspect, the invention provides antibodies that bind to CD3, specifically human CD3E, with a K_{D} of about 100-500 nM, particularly about 150-400 nM, as measured by SPR at 25 °C.

In one aspect, the invention provides an antibody that binds to CD3, wherein the antibody comprises a heavy chain variable region (VH_{CD3}) and a light chain variable region (VL_{CD3}).

### 1. Antibody FV043862

Antibody “FV043862” and its heavy chain variable region (VH_{CD3}) and light chain variable region (VL_{CD3}) are described in the following.

In one aspect, the VH_{CD3} comprises the heavy chain complementarity determining region (HCDR) 1 of SEQ ID NO: 1, the HCDR 2 of SEQ ID NO: 2, and the HCDR 3 of SEQ ID NO: 3.

In one aspect, the antibody is a humanized antibody. In one aspect, the VH_{CD3} is a humanized variable region. In one aspect, the VH_{CD3} comprises an acceptor human framework, e.g. a human immunoglobulin framework or a human consensus framework.

In one aspect, the VH_{CD3} comprises one or more heavy chain framework sequence (i.e. the FR1, FR2, FR3 and/or FR4 sequence) of the heavy chain variable region sequence of SEQ ID NO: 4.

In one aspect, the VH_{CD3} comprises an amino acid sequence having at least 95%, at least 96%, at least 97%, at least 98% or at least 99% sequence identity to the VH sequence of SEQ ID NO: 4. In one aspect, the VH_{CD3} comprises an amino acid sequence having at least 95% sequence identity to the VH sequence of SEQ ID NO: 4. In one aspect, the VH_{CD3} comprises an amino acid sequence having at least 98% sequence identity to the VH sequence of SEQ ID NO: 4.

In one aspect, a VH sequence having at least 95%, 96%, 97%, 98%, or 99% identity contains substitutions (e.g., conservative substitutions), insertions, or deletions relative to the reference sequence, but an antibody (or antigen binding domain) comprising that sequence retains the ability to bind to CD3. In one aspect, a total of 1 to 10 amino acids have been substituted, inserted and/or deleted in the amino acid sequence of SEQ ID NO: 4. In one aspect, substitutions, insertions, or deletions occur in regions outside the CDRs (i.e., in the FRs).

In one aspect, the VH_{CD3} comprises the amino acid sequence of SEQ ID NO: 4 comprising up to two (i.e. 0, 1 or 2) amino acid substitutions within said amino acid sequence. In one aspect, the VH_{CD3} comprises the amino acid sequence of SEQ ID NO: 4 comprising one amino acid substitution within said amino acid sequence. In one aspect, the VH_{CD3} comprises the amino acid sequence of SEQ ID NO: 4 comprising two amino acid substitutions within said amino acid sequence. In one aspect, the substitutions are in the FR of the VH.

In one aspect, the VH_{CD3} comprises the HCDR 1 of SEQ ID NO: 1, the HCDR 2 of SEQ ID NO: 2, and the HCDR 3 of SEQ ID NO: 3, and an amino acid sequence having at least 95%, at least 96%, at least 97%, at least 98% or at least 99% sequence identity to the amino acid sequence of SEQ ID NO: 4. In one aspect, the VH_{CD3} comprises the HCDR 1 of SEQ ID NO: 1, the HCDR 2 of SEQ ID NO: 2, and the HCDR 3 of SEQ ID NO: 3, and an amino acid sequence having at least 95% sequence identity to the amino acid sequence of SEQ ID NO: 4. In one aspect, the VH_{CD3} comprises the HCDR 1 of SEQ ID NO: 1, the HCDR 2 of SEQ ID NO: 2, and the HCDR 3 of SEQ ID NO: 3, and an amino acid sequence having at least 98% sequence identity to the amino acid sequence of SEQ ID NO: 4.

In one aspect, the VH_{CD3} comprises the HCDR 1 of SEQ ID NO: 1, the HCDR 2 of SEQ ID NO: 2, and the HCDR 3 of SEQ ID NO: 3, and the amino acid sequence of SEQ ID NO: 4 comprising up to two (i.e. 0, 1 or 2) amino acid substitutions within (the framework region of) said amino acid sequence. In one aspect, the VH_{CD3} comprises the HCDR 1 of SEQ ID NO: 1, the HCDR 2 of SEQ ID NO: 2, and the HCDR 3 of SEQ ID NO: 3, and the amino acid sequence of SEQ ID NO: 4 comprising one amino acid substitution within (the framework region of) said amino acid sequence. In one aspect, the VH_{CD3} comprises the HCDR 1 of SEQ ID NO: 1, the HCDR 2 of SEQ ID NO: 2, and the HCDR 3 of SEQ ID NO: 3, and the amino acid sequence of SEQ ID NO: 4 comprising two amino acid substitutions within (the framework region of) said amino acid sequence.

In one aspect, the VH_{CD3} comprises the heavy chain CDR sequences (i.e. HCDR1, HCDR2 and HCDR3 amino acid sequences) of the VH of SEQ ID NO: 4.

In one aspect, the VH_{CD3} comprises the heavy chain CDR sequences of the VH of SEQ ID NO: 4, and a framework sequence having at least 95%, at least 96%, at least 97%, at least 98% or at least 99% sequence identity to the framework sequence of the VH of SEQ ID NO: 4. In one aspect, the VH_{CD3} comprises the heavy chain CDR sequences of the VH of SEQ ID NO: 4, and a framework sequence having at least 95% sequence identity to the framework sequence of the VH of SEQ ID NO: 4. In one aspect, the VH_{CD3} comprises the heavy chain CDR sequences of the VH of SEQ ID NO: 4, and a framework sequence having at least 98% sequence identity to the framework sequence of the VH of SEQ ID NO: 4.

In one aspect, the VH_{CD3} comprises the heavy chain CDR sequences of the VH of SEQ ID NO: 4, and the framework sequence of the VH of SEQ ID NO: 4 comprising up to two (i.e. 0, 1 or 2) amino acid substitutions within said framework sequence. In one aspect, the VH_{CD3} comprises the heavy chain CDR sequences of the VH of SEQ ID NO: 4, and the framework sequence of the VH of SEQ ID NO: 4 comprising one amino acid substitution within said framework sequence. In one aspect, the VH_{CD3} comprises the heavy chain CDR sequences of the VH of SEQ ID NO: 4, and the framework sequence of the VH of SEQ ID NO: 4 comprising two amino acid substitutions within said framework sequence.

Preferably, the CDR sequences of the VH according to the above aspects are according to the Kabat definition. Alternatively, the CDRs of the VH according to the above aspects are according to the Chothia definition. Further alternatively, the CDRs of the VH according to the above aspects are according to the Contact definition. Still further alternatively, the CDRs of the VH according to the above aspects are according to the IMGT definition.

In one aspect, the VH_{CD3} comprises the amino acid sequence of SEQ ID NO: 4. Optionally, the VH_{CD3} comprises the amino acid sequence of SEQ ID NO: 4, including post-translational modifications of that sequence.

In one aspect, the VL_{CD3} comprises the light chain complementarity determining region (LCDR) 1 of SEQ ID NO: 5, the LCDR 2 of SEQ ID NO: 6, and the LCDR 3 of SEQ ID NO: 7.

In one aspect, the antibody is a humanized antibody. In one aspect, the VL_{CD3} is a humanized variable region. In one aspect, the VL_{CD3} comprises an acceptor human framework, e.g. a human immunoglobulin framework or a human consensus framework.

In some aspects, the VL_{CD3} comprises one or more light chain framework sequence (i.e. the FR1, FR2, FR3 and/or FR4 sequence) of the light chain variable region sequence of SEQ ID NO: 8.

In one aspect, the VL_{CD3} comprises an amino acid sequence having at least 95%, at least 96%, at least 97%, at least 98% or at least 99% sequence identity to the VL sequence of SEQ ID NO: 8. In one aspect, the VL_{CD3} comprises an amino acid sequence having at least 95% sequence identity to the VL sequence of SEQ ID NO: 8. In one aspect, the VL_{CD3} comprises an amino acid sequence having at least 98% sequence identity to the VL sequence of SEQ ID NO: 8.

In one aspect, a VL sequence having at least 95%, 96%, 97%, 98%, or 99% identity contains substitutions (e.g., conservative substitutions), insertions, or deletions relative to the reference sequence, but an antibody (or antigen binding domain) comprising that sequence retains the ability to bind to CD3. In one aspect, a total of 1 to 10 amino acids have been substituted, inserted and/or deleted in the amino acid sequence of SEQ ID NO: 8. In one aspect, substitutions, insertions, or deletions occur in regions outside the CDRs (i.e., in the FRs).

In one aspect, the VL_{CD3} comprises the amino acid sequence of SEQ ID NO: 8 comprising up to two (i.e. 0, 1 or 2) amino acid substitutions within said amino acid sequence. In one aspect, the VL_{CD3} comprises the amino acid sequence of SEQ ID NO: 8 comprising one amino acid substitution within said amino acid sequence. In one aspect, the VL_{CD3} comprises the amino acid sequence of SEQ ID NO: 8 comprising two amino acid substitutions within said amino acid sequence. In one aspect, the substitutions are in the FR of the VL.

In one aspect, the VL_{CD3} comprises the LCDR 1 of SEQ ID NO: 5, the LCDR 2 of SEQ ID NO: 6, and the LCDR 3 of SEQ ID NO: 7, and an amino acid sequence having at least 95%, at least 96%, at least 97%, at least 98% or at least 99% sequence identity to the amino acid sequence of SEQ ID NO: 8. In one aspect, the VL_{CD3} comprises the LCDR 1 of SEQ ID NO: 5, the LCDR 2 of SEQ ID NO: 6, and the LCDR 3 of SEQ ID NO: 7, and an amino acid sequence having at least 95% sequence identity to the amino acid sequence of SEQ ID NO: 8. In one aspect, the VL_{CD3} comprises the LCDR 1 of SEQ ID NO: 5, the LCDR 2 of SEQ ID NO: 6, and the LCDR 3 of SEQ ID NO: 7, and an amino acid sequence having at least 98% sequence identity to the amino acid sequence of SEQ ID NO: 8.

In one aspect, the VL_{CD3} comprises the LCDR 1 of SEQ ID NO: 5, the LCDR 2 of SEQ ID NO: 6, and the LCDR 3 of SEQ ID NO: 7, and the amino acid sequence of SEQ ID NO: 8 comprising up to two (i.e. 0, 1 or 2) amino acid substitutions within (the framework region of) said amino acid sequence. In one aspect, the VL_{CD3} comprises the LCDR 1 of SEQ ID NO: 5, the LCDR 2 of SEQ ID NO: 6, and the LCDR 3 of SEQ ID NO: 7, and the amino acid sequence of SEQ ID NO: 8 comprising one amino acid substitution within (the framework region of) said amino acid sequence. In one aspect, the VL_{CD3} comprises the LCDR 1 of SEQ ID NO: 5, the LCDR 2 of SEQ ID NO: 6, and the LCDR 3 of SEQ ID NO: 7, and the amino acid sequence of SEQ ID NO: 8 comprising two amino acid substitutions within (the framework region of) said amino acid sequence.

In one aspect, the VL_{CD3} comprises the light chain CDR sequences (i.e. LCDR1, LCDR2 and LCDR3 amino acid sequences) of the VL of SEQ ID NO: 8.

In one aspect, the VL_{CD3} comprises the light chain CDR sequences of the VL of SEQ ID NO: 8, and a framework sequence having at least 95%, at least 96%, at least 97%, at least 98% or at least 99% sequence identity to the framework sequence of the VL of SEQ ID NO: 8. In one aspect, the VL_{CD3} comprises the light chain CDR sequences of the VL of SEQ ID NO: 8, and a framework sequence having at least 95% sequence identity to the framework sequence of the VL of SEQ ID NO: 8. In one aspect, the VL_{CD3} comprises the light chain CDR sequences of the VL of SEQ ID NO: 8, and a framework sequence having at least 98% sequence identity to the framework sequence of the VL of SEQ ID NO: 8.

In one aspect, the VL_{CD3} comprises the light chain CDR sequences of the VL of SEQ ID NO: 8, and the framework sequence of the VH of SEQ ID NO: 8 comprising up to two (i.e. 0, 1 or 2) amino acid substitutions within said framework sequence. In one aspect, the VL_{CD3} comprises the light chain CDR sequences of the VL of SEQ ID NO: 8, and the framework sequence of the VL of SEQ ID NO: 8 comprising one amino acid substitution within said framework sequence. In one aspect, the VL_{CD3} comprises the light chain CDR sequences of the VL of SEQ ID NO: 8, and the framework sequence of the VL of SEQ ID NO: 8 comprising two amino acid substitutions within said framework sequence.

Preferably, the CDR sequences of the VL according to the above aspects are according to the Kabat definition. Alternatively, the CDRs of the VL according to the above aspects are according to the Chothia definition. Further alternatively, the CDRs of the VL according to the above aspects are according to the Contact definition. Still further alternatively, the CDRs of the VL according to the above aspects are according to the IMGT definition.

In one aspect, the VL_{CD3} comprises the amino acid sequence of SEQ ID NO: 8. Optionally, the VL_{CD3} comprises the amino acid sequence of SEQ ID NO: 8, including post-translational modifications of that sequence.

In particular aspects, the antibody (or the first antigen binding domain, as described herein) comprises a VH sequence (VH_{CD3}) as in any of the aspects provided above, and a VL sequence (VL_{CD3}) as in any of the aspects provided above.

In one aspect, the antibody (or the first antigen binding domain, as described herein) comprises a heavy chain variable region (VH_{CD3}) comprising the HCDR 1 of SEQ ID NO: 1, the HCDR 2 of SEQ ID NO: 2, and the HCDR 3 of SEQ ID NO: 3; and a light chain variable region (VL_{CD3}) comprising the LCDR 1 of SEQ ID NO: 5, the LCDR 2 of SEQ ID NO: 6 and the LCDR 3 of SEQ ID NO: 7.

In one aspect, the antibody (or the first antigen binding domain, as described herein) comprises a heavy chain variable region (VH_{CD3}) comprising an amino acid sequence having at least 95%, at least 96%, at least 97%, at least 98% or at least 99% sequence identity to the VH sequence of SEQ ID NO: 4; and a light chain variable region (VL_{CD3}) comprising an amino acid sequence having at least 95%, at least 96%, at least 97%, at least 98% or at least 99% sequence identity to the VL sequence of SEQ ID NO: 8.

In one aspect, the antibody (or the first antigen binding domain, as described herein) comprises a heavy chain variable region (VH_{CD3}) comprising the amino acid sequence of SEQ ID NO: 4 comprising up to two (i.e. 0, 1 or 2) amino acid substitutions within said amino acid sequence; and a light chain variable region (VL_{CD3}) comprising the amino acid sequence of SEQ ID NO: 8 comprising up to two (i.e. 0, 1 or 2) amino acid substitutions within said amino acid sequence.

In one aspect, the antibody (or the first antigen binding domain, as described herein) comprises a heavy chain variable region (VH_{CD3}) comprising the HCDR 1 of SEQ ID NO: 1, the HCDR 2 of SEQ ID NO: 2, and the HCDR 3 of SEQ ID NO: 3, and an amino acid sequence having at least 95%, at least 96%, at least 97%, at least 98% or at least 99% sequence identity to the amino acid sequence of SEQ ID NO: 4; and a light chain variable region (VL_{CD3}) comprising the LCDR 1 of SEQ ID NO: 5, the LCDR 2 of SEQ ID NO: 6, and the LCDR 3 of SEQ ID NO: 7, and an amino acid sequence having at least 95%, at least 96%, at least 97%, at least 98% or at least 99% sequence identity to the amino acid sequence of SEQ ID NO: 8.

In one aspect, the antibody (or the first antigen binding domain, as described herein) comprises a heavy chain variable region (VH_{CD3}) comprising the HCDR 1 of SEQ ID NO: 1, the HCDR 2 of SEQ ID NO: 2, and the HCDR 3 of SEQ ID NO: 3, and the amino acid sequence of SEQ ID NO: 4 comprising up to two (i.e. 0, 1 or 2) amino acid substitutions within (the framework region of) said amino acid sequence; and a light chain variable region (VL_{CD3}) comprising the LCDR 1 of SEQ ID NO: 5, the LCDR 2 of SEQ ID NO: 6, and the LCDR 3 of SEQ ID NO: 7, and the amino acid sequence of SEQ ID NO: 8 comprising up to two (i.e. 0, 1 or 2) amino acid substitutions within (the framework region of) said amino acid sequence.

In one aspect, the antibody (or the first antigen binding domain, as described herein) comprises a heavy chain variable region (VH_{CD3}) comprising the heavy chain CDR sequences (i.e. HCDR1, HCDR2 and HCDR3 amino acid sequences) of the VH of SEQ ID NO: 4; and a light chain variable region (VL_{CD3}) comprising the light chain CDR sequences (i.e. LCDR1, LCDR2 and LCDR3 amino acid sequences) of the VL of SEQ ID NO: 8.

In one aspect, the antibody (or the first antigen binding domain, as described herein) comprises a heavy chain variable region (VH_{CD3}) comprising the heavy chain CDR sequences of the VH of SEQ ID NO: 4, and a framework sequence having at least 95%, at least 96%, at least 97%, at least 98% or at least 99% sequence identity to the framework sequence of the VH of SEQ ID NO: 4; and a light chain variable region (VL_{CD3}) comprising the light chain CDR sequences of the VL of SEQ ID NO: 8, and a framework sequence having at least 95%, at least 96%, at least 97%, at least 98% or at least 99% sequence identity to the framework sequence of the VL of SEQ ID NO: 8.

In one aspect, the antibody (or the first antigen binding domain, as described herein) comprises a heavy chain variable region (VH_{CD3}) comprising the heavy chain CDR sequences of the VH of SEQ ID NO: 4, and the framework sequence of the VH of SEQ ID NO: 4 comprising up to two (i.e. 0, 1 or 2) amino acid substitutions within said framework sequence; and a light chain variable region (VL_{CD3}) comprising the light chain CDR sequences of the VL of SEQ ID NO: 8, and the framework sequence of the VL of SEQ ID NO: 8 comprising up to two (i.e. 0, 1 or 2) amino acid substitutions within said framework sequence.

Preferably, the CDR sequences of the VH and/or VL according to the above aspects are according to the Kabat definition. Alternatively, the CDRs of the VH and/or VL according to the above aspects are according to the Chothia definition. Further alternatively, the CDRs of the VH and/or VL according to the above aspects are according to the Contact definition. Still further alternatively, the CDRs of the VH and/or VL according to the above aspects are according to the IMGT definition.

In one aspect, the antibody (or the first antigen binding domain, as described herein) comprises a heavy chain variable region (VH_{CD3}) comprising the amino acid sequence of SEQ ID NO: 4; and a light chain variable region (VL_{CD3}) comprising the amino acid sequence of SEQ ID NO: 8.

### Antibody FV043861

Antibody “FV043861” and its heavy chain variable region (VH_{CD3}) and light chain variable region (VL_{CD3}) are described in the following.

In one aspect, the VH_{CD3} comprises the heavy chain complementarity determining region (HCDR) 1 of SEQ ID NO: 1, the HCDR 2 of SEQ ID NO: 9, and the HCDR 3 of SEQ ID NO: 10.

In one aspect, the antibody is a humanized antibody. In one aspect, the VH_{CD3} is a humanized variable region. In one aspect, the VH_{CD3} comprises an acceptor human framework, e.g. a human immunoglobulin framework or a human consensus framework.

In some aspects, the VH_{CD3} comprises one or more heavy chain framework sequence (i.e. the FR1, FR2, FR3 and/or FR4 sequence) of the heavy chain variable region sequence of SEQ ID NO: 11.

In one aspect, the VH_{CD3} comprises an amino acid sequence having at least 95%, at least 96%, at least 97%, at least 98% or at least 99% sequence identity to the VH sequence of SEQ ID NO: 11. In one aspect, the VH_{CD3} comprises an amino acid sequence having at least 95% sequence identity to the VH sequence of SEQ ID NO: 11. In one aspect, the VH_{CD3} comprises an amino acid sequence having at least 98% sequence identity to the VH sequence of SEQ ID NO: 11.

In one aspect, a VH sequence having at least 95%, 96%, 97%, 98%, or 99% identity contains substitutions (e.g., conservative substitutions), insertions, or deletions relative to the reference sequence, but an antibody (or antigen binding domain) comprising that sequence retains the ability to bind to CD3. In one aspect, a total of 1 to 10 amino acids have been substituted, inserted and/or deleted in the amino acid sequence of SEQ ID NO: 11. In one aspect, substitutions, insertions, or deletions occur in regions outside the CDRs (i.e., in the FRs).

In one aspect, the VH_{CD3} comprises the amino acid sequence of SEQ ID NO: 11 comprising up to two (i.e. 0, 1 or 2) amino acid substitutions within said amino acid sequence. In one aspect, the VH_{CD3} comprises the amino acid sequence of SEQ ID NO: 11 comprising one amino acid substitution within said amino acid sequence. In one aspect, the VH_{CD3} comprises the amino acid sequence of SEQ ID NO: 11 comprising two amino acid substitutions within said amino acid sequence. In one aspect, the substitutions are in the FR of the VH.

In one aspect, the VH_{CD3} comprises the HCDR 1 of SEQ ID NO: 1, the HCDR 2 of SEQ ID NO: 9, and the HCDR 3 of SEQ ID NO: 10, and an amino acid sequence having at least 95%, at least 96%, at least 97%, at least 98% or at least 99% sequence identity to the amino acid sequence of SEQ ID NO: 11. In one aspect, the VH_{CD3} comprises the HCDR 1 of SEQ ID NO: 1, the HCDR 2 of SEQ ID NO: 9, and the HCDR 3 of SEQ ID NO: 10, and an amino acid sequence having at least 95% sequence identity to the amino acid sequence of SEQ ID NO: 11. In one aspect, the VH_{CD3} comprises the HCDR 1 of SEQ ID NO: 1, the HCDR 2 of SEQ ID NO: 9, and the HCDR 3 of SEQ ID NO: 10, and an amino acid sequence having at least 98% sequence identity to the amino acid sequence of SEQ ID NO: 11.

In one aspect, the VH_{CD3} comprises the HCDR 1 of SEQ ID NO: 1, the HCDR 2 of SEQ ID NO: 9, and the HCDR 3 of SEQ ID NO: 10, and the amino acid sequence of SEQ ID NO: 11 comprising up to two (i.e. 0, 1 or 2) amino acid substitutions within (the framework region of) said amino acid sequence. In one aspect, the VH_{CD3} comprises the HCDR 1 of SEQ ID NO: 1, the HCDR 2 of SEQ ID NO: 9, and the HCDR 3 of SEQ ID NO: 10, and the amino acid sequence of SEQ ID NO: 11 comprising one amino acid substitution within (the framework region of) said amino acid sequence. In one aspect, the VH_{CD3} comprises the HCDR 1 of SEQ ID NO: 1, the HCDR 2 of SEQ ID NO: 9, and the HCDR 3 of SEQ ID NO: 10, and the amino acid sequence of SEQ ID NO: 11 comprising two amino acid substitutions within (the framework region of) said amino acid sequence.

In one aspect, the VH_{CD3} comprises the heavy chain CDR sequences (i.e. HCDR1, HCDR2 and HCDR3 amino acid sequences) of the VH of SEQ ID NO: 11.

In one aspect, the VH_{CD3} comprises the heavy chain CDR sequences of the VH of SEQ ID NO: 11, and a framework sequence having at least 95%, at least 96%, at least 97%, at least 98% or at least 99% sequence identity to the framework sequence of the VH of SEQ ID NO: 11. In one aspect, the VH_{CD3} comprises the heavy chain CDR sequences of the VH of SEQ ID NO: 11, and a framework sequence having at least 95% sequence identity to the framework sequence of the VH of SEQ ID NO: 11. In one aspect, the VH_{CD3} comprises the heavy chain CDR sequences of the VH of SEQ ID NO: 11, and a framework sequence having at least 98% sequence identity to the framework sequence of the VH of SEQ ID NO: 11.

In one aspect, the VH_{CD3} comprises the heavy chain CDR sequences of the VH of SEQ ID NO: 11, and the framework sequence of the VH of SEQ ID NO: 11 comprising up to two (i.e. 0, 1 or 2) amino acid substitutions within said framework sequence. In one aspect, the VH_{CD3} comprises the heavy chain CDR sequences of the VH of SEQ ID NO: 11, and the framework sequence of the VH of SEQ ID NO: 11 comprising one amino acid substitution within said framework sequence. In one aspect, the VH_{CD3} comprises the heavy chain CDR sequences of the VH of SEQ ID NO: 11, and the framework sequence of the VH of SEQ ID NO: 11 comprising two amino acid substitutions within said framework sequence.

Preferably, the CDR sequences of the VH according to the above aspects are according to the Kabat definition. Alternatively, the CDRs of the VH according to the above aspects are according to the Chothia definition. Further alternatively, the CDRs of the VH according to the above aspects are according to the Contact definition. Still further alternatively, the CDRs of the VH according to the above aspects are according to the IMGT definition.

In one aspect, the VH_{CD3} comprises the amino acid sequence of SEQ ID NO: 11. Optionally, the VH_{CD3} comprises the amino acid sequence of SEQ ID NO: 11, including post-translational modifications of that sequence.

In one aspect, the VL_{CD3} comprises the light chain complementarity determining region (LCDR) 1 of SEQ ID NO: 5, the LCDR 2 of SEQ ID NO: 6, and the LCDR 3 of SEQ ID NO: 7.

In one aspect, the antibody is a humanized antibody. In one aspect, the VL_{CD3} is a humanized variable region. In one aspect, the VL_{CD3} comprises an acceptor human framework, e.g. a human immunoglobulin framework or a human consensus framework.

In some aspects, the VL_{CD3} comprises one or more light chain framework sequence (i.e. the FR1, FR2, FR3 and/or FR4 sequence) of the light chain variable region sequence of SEQ ID NO: 8.

In one aspect, the VL_{CD3} comprises an amino acid sequence having at least 95%, at least 96%, at least 97%, at least 98% or at least 99% sequence identity to the VL sequence of SEQ ID NO: 8. In one aspect, the VL_{CD3} comprises an amino acid sequence having at least 95% sequence identity to the VL sequence of SEQ ID NO: 8. In one aspect, the VL_{CD3} comprises an amino acid sequence having at least 98% sequence identity to the VL sequence of SEQ ID NO: 8.

In one aspect, a VL sequence having at least 95%, 96%, 97%, 98%, or 99% identity contains substitutions (e.g., conservative substitutions), insertions, or deletions relative to the reference sequence, but an antibody (or antigen binding domain) comprising that sequence retains the ability to bind to CD3. In one aspect, a total of 1 to 10 amino acids have been substituted, inserted and/or deleted in the amino acid sequence of SEQ ID NO: 8. In one aspect, substitutions, insertions, or deletions occur in regions outside the CDRs (i.e., in the FRs).

In one aspect, the VL_{CD3} comprises the amino acid sequence of SEQ ID NO: 8 comprising up to two (i.e. 0, 1 or 2) amino acid substitutions within said amino acid sequence. In one aspect, the VL_{CD3} comprises the amino acid sequence of SEQ ID NO: 8 comprising one amino acid substitution within said amino acid sequence. In one aspect, the VL_{CD3} comprises the amino acid sequence of SEQ ID NO: 8 comprising two amino acid substitutions within said amino acid sequence. In one aspect, the substitutions are in the FR of the VL.

In one aspect, the VL_{CD3} comprises the LCDR 1 of SEQ ID NO: 5, the LCDR 2 of SEQ ID NO: 6, and the LCDR 3 of SEQ ID NO: 7, and an amino acid sequence having at least 95%, at least 96%, at least 97%, at least 98% or at least 99% sequence identity to the amino acid sequence of SEQ ID NO: 8. In one aspect, the VL_{CD3} comprises the LCDR 1 of SEQ ID NO: 5, the LCDR 2 of SEQ ID NO: 6, and the LCDR 3 of SEQ ID NO: 7, and an amino acid sequence having at least 95% sequence identity to the amino acid sequence of SEQ ID NO: 8. In one aspect, the VL_{CD3} comprises the LCDR 1 of SEQ ID NO: 5, the LCDR 2 of SEQ ID NO: 6, and the LCDR 3 of SEQ ID NO: 7, and an amino acid sequence having at least 98% sequence identity to the amino acid sequence of SEQ ID NO: 8.

In one aspect, the VL_{CD3} comprises the LCDR 1 of SEQ ID NO: 5, the LCDR 2 of SEQ ID NO: 6, and the LCDR 3 of SEQ ID NO: 7, and the amino acid sequence of SEQ ID NO: 8 comprising up to two (i.e. 0, 1 or 2) amino acid substitutions within (the framework region of) said amino acid sequence. In one aspect, the VL_{CD3} comprises the LCDR 1 of SEQ ID NO: 5, the LCDR 2 of SEQ ID NO: 6, and the LCDR 3 of SEQ ID NO: 7, and the amino acid sequence of SEQ ID NO: 8 comprising one amino acid substitution within (the framework region of) said amino acid sequence. In one aspect, the VL_{CD3} comprises the LCDR 1 of SEQ ID NO: 5, the LCDR 2 of SEQ ID NO: 6, and the LCDR 3 of SEQ ID NO: 7, and the amino acid sequence of SEQ ID NO: 8 comprising two amino acid substitutions within (the framework region of) said amino acid sequence.

In one aspect, the VL_{CD3} comprises the light chain CDR sequences (i.e. LCDR1, LCDR2 and LCDR3 amino acid sequences) of the VL of SEQ ID NO: 8.

In one aspect, the VL_{CD3} comprises the light chain CDR sequences of the VL of SEQ ID NO: 8, and a framework sequence having at least 95%, at least 96%, at least 97%, at least 98% or at least 99% sequence identity to the framework sequence of the VL of SEQ ID NO: 8. In one aspect, the VL_{CD3} comprises the light chain CDR sequences of the VL of SEQ ID NO: 8, and a framework sequence having at least 95% sequence identity to the framework sequence of the VL of SEQ ID NO: 8. In one aspect, the VL_{CD3} comprises the light chain CDR sequences of the VL of SEQ ID NO: 8, and a framework sequence having at least 98% sequence identity to the framework sequence of the VL of SEQ ID NO: 8.

In one aspect, the VL_{CD3} comprises the light chain CDR sequences of the VL of SEQ ID NO: 8, and the framework sequence of the VH of SEQ ID NO: 8 comprising up to two (i.e. 0, 1 or 2) amino acid substitutions within said framework sequence. In one aspect, the VL_{CD3} comprises the light chain CDR sequences of the VL of SEQ ID NO: 8, and the framework sequence of the VL of SEQ ID NO: 8 comprising one amino acid substitution within said framework sequence. In one aspect, the VL_{CD3} comprises the light chain CDR sequences of the VL of SEQ ID NO: 8, and the framework sequence of the VL of SEQ ID NO: 8 comprising two amino acid substitutions within said framework sequence.

Preferably, the CDR sequences of the VL according to the above aspects are according to the Kabat definition. Alternatively, the CDRs of the VL according to the above aspects are according to the Chothia definition. Further alternatively, the CDRs of the VL according to the above aspects are according to the Contact definition. Still further alternatively, the CDRs of the VL according to the above aspects are according to the IMGT definition.

In one aspect, the VL_{CD3} comprises the amino acid sequence of SEQ ID NO: 8. Optionally, the VL_{CD3} comprises the amino acid sequence of SEQ ID NO: 8, including post-translational modifications of that sequence.

In particular aspects, the antibody (or the first antigen binding domain, as described herein) comprises a VH sequence (VH_{CD3}) as in any of the aspects provided above, and a VL sequence (VL_{CD3}) as in any of the aspects provided above.

In one aspect, the antibody (or the first antigen binding domain, as described herein) comprises a heavy chain variable region (VH_{CD3}) comprising the HCDR 1 of SEQ ID NO: 1, the HCDR 2 of SEQ ID NO: 9, and the HCDR 3 of SEQ ID NO: 10; and a light chain variable region (VL_{CD3}) comprising the LCDR 1 of SEQ ID NO: 5, the LCDR 2 of SEQ ID NO: 6 and the LCDR 3 of SEQ ID NO: 7.

In one aspect, the antibody (or the first antigen binding domain, as described herein) comprises a heavy chain variable region (VH_{CD3}) comprising an amino acid sequence having at least 95%, at least 96%, at least 97%, at least 98% or at least 99% sequence identity to the VH sequence of SEQ ID NO: 11; and a light chain variable region (VL_{CD3}) comprising an amino acid sequence having at least 95%, at least 96%, at least 97%, at least 98% or at least 99% sequence identity to the VL sequence of SEQ ID NO: 8.

In one aspect, the antibody (or the first antigen binding domain, as described herein) comprises a heavy chain variable region (VH_{CD3}) comprising the amino acid sequence of SEQ ID NO: 11 comprising up to two (i.e. 0, 1 or 2) amino acid substitutions within said amino acid sequence; and a light chain variable region (VL_{CD3}) comprising the amino acid sequence of SEQ ID NO: 8 comprising up to two (i.e. 0, 1 or 2) amino acid substitutions within said amino acid sequence.

In one aspect, the antibody (or the first antigen binding domain, as described herein) comprises a heavy chain variable region (VH_{CD3}) comprising the HCDR 1 of SEQ ID NO: 1, the HCDR 2 of SEQ ID NO: 9, and the HCDR 3 of SEQ ID NO: 10, and an amino acid sequence having at least 95%, at least 96%, at least 97%, at least 98% or at least 99% sequence identity to the amino acid sequence of SEQ ID NO: 11; and a light chain variable region (VL_{CD3}) comprising the LCDR 1 of SEQ ID NO: 5, the LCDR 2 of SEQ ID NO: 6, and the LCDR 3 of SEQ ID NO: 7, and an amino acid sequence having at least 95%, at least 96%, at least 97%, at least 98% or at least 99% sequence identity to the amino acid sequence of SEQ ID NO: 8.

In one aspect, the antibody (or the first antigen binding domain, as described herein) comprises a heavy chain variable region (VH_{CD3}) comprising the HCDR 1 of SEQ ID NO: 1, the HCDR 2 of SEQ ID NO: 9, and the HCDR 3 of SEQ ID NO: 10, and the amino acid sequence of SEQ ID NO: 11 comprising up to two (i.e. 0, 1 or 2) amino acid substitutions within (the framework region of) said amino acid sequence; and a light chain variable region (VL_{CD3}) comprising the LCDR 1 of SEQ ID NO: 5, the LCDR 2 of SEQ ID NO: 6, and the LCDR 3 of SEQ ID NO: 7, and the amino acid sequence of SEQ ID NO: 8 comprising up to two (i.e. 0, 1 or 2) amino acid substitutions within (the framework region of) said amino acid sequence.

In one aspect, the antibody (or the first antigen binding domain, as described herein) comprises a heavy chain variable region (VH_{CD3}) comprising the heavy chain CDR sequences (i.e. HCDR1, HCDR2 and HCDR3 amino acid sequences) of the VH of SEQ ID NO: 11; and a light chain variable region (VL_{CD3}) comprising the light chain CDR sequences (i.e. LCDR1, LCDR2 and LCDR3 amino acid sequences) of the VL of SEQ ID NO: 8.

In one aspect, the antibody (or the first antigen binding domain, as described herein) comprises a heavy chain variable region (VH_{CD3}) comprising the heavy chain CDR sequences of the VH of SEQ ID NO: 11, and a framework sequence having at least 95%, at least 96%, at least 97%, at least 98% or at least 99% sequence identity to the framework sequence of the VH of SEQ ID NO: 11; and a light chain variable region (VL_{CD3}) comprising the light chain CDR sequences of the VL of SEQ ID NO: 8, and a framework sequence having at least 95%, at least 96%, at least 97%, at least 98% or at least 99% sequence identity to the framework sequence of the VL of SEQ ID NO: 8.

In one aspect, the antibody (or the first antigen binding domain, as described herein) comprises a heavy chain variable region (VH_{CD3}) comprising the heavy chain CDR sequences of the VH of SEQ ID NO: 11, and the framework sequence of the VH of SEQ ID NO: 11 comprising up to two (i.e. 0, 1 or 2) amino acid substitutions within said framework sequence; and a light chain variable region (VL_{CD3}) comprising the light chain CDR sequences of the VL of SEQ ID NO: 8, and the framework sequence of the VL of SEQ ID NO: 8 comprising up to two (i.e. 0, 1 or 2) amino acid substitutions within said framework sequence.

Preferably, the CDR sequences of the VH and/or VL according to the above aspects are according to the Kabat definition. Alternatively, the CDRs of the VH and/or VL according to the above aspects are according to the Chothia definition. Further alternatively, the CDRs of the VH and/or VL according to the above aspects are according to the Contact definition. Still further alternatively, the CDRs of the VH and/or VL according to the above aspects are according to the IMGT definition.

In one aspect, the antibody (or the first antigen binding domain, as described herein) comprises a heavy chain variable region (VH_{CD3}) comprising the amino acid sequence of SEQ ID NO: 11; and a light chain variable region (VL_{CD3}) comprising the amino acid sequence of SEQ ID NO: 8.

### 2. Antibody FV032417

Antibody “FV032417” and its heavy chain variable region (VH_{CD3}) and light chain variable region (VL_{CD3}) are described in the following.

In one aspect, the VH_{CD3} comprises the heavy chain complementarity determining region (HCDR) 1 of SEQ ID NO: 1, the HCDR 2 of SEQ ID NO: 2, and the HCDR 3 of SEQ ID NO: 10.

In one aspect, the antibody is a humanized antibody. In one aspect, the VH_{CD3} is a humanized variable region. In one aspect, the VH_{CD3} comprises an acceptor human framework, e.g. a human immunoglobulin framework or a human consensus framework.

In some aspects, the VH_{CD3} comprises one or more heavy chain framework sequence (i.e. the FR1, FR2, FR3 and/or FR4 sequence) of the heavy chain variable region sequence of SEQ ID NO: 12.

In one aspect, the VH_{CD3} comprises an amino acid sequence having at least 95%, at least 96%, at least 97%, at least 98% or at least 99% sequence identity to the VH sequence of SEQ ID NO: 12. In one aspect, the VH_{CD3} comprises an amino acid sequence having at least 95% sequence identity to the VH sequence of SEQ ID NO: 12. In one aspect, the VH_{CD3} comprises an amino acid sequence having at least 98% sequence identity to the VH sequence of SEQ ID NO: 12.

In one aspect, a VH sequence having at least 95%, 96%, 97%, 98%, or 99% identity contains substitutions (e.g., conservative substitutions), insertions, or deletions relative to the reference sequence, but an antibody (or antigen binding domain) comprising that sequence retains the ability to bind to CD3. In one aspect, a total of 1 to 10 amino acids have been substituted, inserted and/or deleted in the amino acid sequence of SEQ ID NO: 12. In one aspect, substitutions, insertions, or deletions occur in regions outside the CDRs (i.e., in the FRs).

In one aspect, the VH_{CD3} comprises the amino acid sequence of SEQ ID NO: 12 comprising up to two (i.e. 0, 1 or 2) amino acid substitutions within said amino acid sequence. In one aspect, the VH_{CD3} comprises the amino acid sequence of SEQ ID NO: 12 comprising one amino acid substitution within said amino acid sequence. In one aspect, the VH_{CD3} comprises the amino acid sequence of SEQ ID NO: 12 comprising two amino acid substitutions within said amino acid sequence. In one aspect, the substitutions are in the FR of the VH.

In one aspect, the VH_{CD3} comprises the HCDR 1 of SEQ ID NO: 1, the HCDR 2 of SEQ ID NO: 2, and the HCDR 3 of SEQ ID NO: 10, and an amino acid sequence having at least 95%, at least 96%, at least 97%, at least 98% or at least 99% sequence identity to the amino acid sequence of SEQ ID NO: 12. In one aspect, the VH_{CD3} comprises the HCDR 1 of SEQ ID NO: 1, the HCDR 2 of SEQ ID NO: 2, and the HCDR 3 of SEQ ID NO: 10, and an amino acid sequence having at least 95% sequence identity to the amino acid sequence of SEQ ID NO: 12. In one aspect, the VH_{CD3} comprises the HCDR 1 of SEQ ID NO: 1, the HCDR 2 of SEQ ID NO: 2, and the HCDR 3 of SEQ ID NO: 10, and an amino acid sequence having at least 98% sequence identity to the amino acid sequence of SEQ ID NO: 12.

In one aspect, the VH_{CD3} comprises the HCDR 1 of SEQ ID NO: 1, the HCDR 2 of SEQ ID NO: 2, and the HCDR 3 of SEQ ID NO: 10, and the amino acid sequence of SEQ ID NO: 12 comprising up to two (i.e. 0, 1 or 2) amino acid substitutions within (the framework region of) said amino acid sequence. In one aspect, the VH_{CD3} comprises the HCDR 1 of SEQ ID NO: 1, the HCDR 2 of SEQ ID NO: 2, and the HCDR 3 of SEQ ID NO: 10, and the amino acid sequence of SEQ ID NO: 12 comprising one amino acid substitution within (the framework region of) said amino acid sequence. In one aspect, the VH_{CD3} comprises the HCDR 1 of SEQ ID NO: 1, the HCDR 2 of SEQ ID NO: 2, and the HCDR 3 of SEQ ID NO: 10, and the amino acid sequence of SEQ ID NO: 12 comprising two amino acid substitutions within (the framework region of) said amino acid sequence.

In one aspect, the VH_{CD3} comprises the heavy chain CDR sequences (i.e. HCDR1, HCDR2 and HCDR3 amino acid sequences) of the VH of SEQ ID NO: 12.

In one aspect, the VH_{CD3} comprises the heavy chain CDR sequences of the VH of SEQ ID NO: 12, and a framework sequence having at least 95%, at least 96%, at least 97%, at least 98% or at least 99% sequence identity to the framework sequence of the VH of SEQ ID NO: 12. In one aspect, the VH_{CD3} comprises the heavy chain CDR sequences of the VH of SEQ ID NO: 12, and a framework sequence having at least 95% sequence identity to the framework sequence of the VH of SEQ ID NO: 12. In one aspect, the VH_{CD3} comprises the heavy chain CDR sequences of the VH of SEQ ID NO: 12, and a framework sequence having at least 98% sequence identity to the framework sequence of the VH of SEQ ID NO: 12.

In one aspect, the VH_{CD3} comprises the heavy chain CDR sequences of the VH of SEQ ID NO: 12, and the framework sequence of the VH of SEQ ID NO: 12 comprising up to two (i.e. 0, 1 or 2) amino acid substitutions within said framework sequence. In one aspect, the VH_{CD3} comprises the heavy chain CDR sequences of the VH of SEQ ID NO: 12, and the framework sequence of the VH of SEQ ID NO: 12 comprising one amino acid substitution within said framework sequence. In one aspect, the VH_{CD3} comprises the heavy chain CDR sequences of the VH of SEQ ID NO: 12, and the framework sequence of the VH of SEQ ID NO: 12 comprising two amino acid substitutions within said framework sequence.

Preferably, the CDR sequences of the VH according to the above aspects are according to the Kabat definition. Alternatively, the CDRs of the VH according to the above aspects are according to the Chothia definition. Further alternatively, the CDRs of the VH according to the above aspects are according to the Contact definition. Still further alternatively, the CDRs of the VH according to the above aspects are according to the IMGT definition.

In one aspect, the VH_{CD3} comprises the amino acid sequence of SEQ ID NO: 12. Optionally, the VH_{CD3} comprises the amino acid sequence of SEQ ID NO: 12, including post-translational modifications of that sequence.

In one aspect, the VL_{CD3} comprises the light chain complementarity determining region (LCDR) 1 of SEQ ID NO: 5, the LCDR 2 of SEQ ID NO: 6, and the LCDR 3 of SEQ ID NO: 13.

In one aspect, the antibody is a humanized antibody. In one aspect, the VL_{CD3} is a humanized variable region. In one aspect, the VL_{CD3} comprises an acceptor human framework, e.g. a human immunoglobulin framework or a human consensus framework.

In some aspects, the VL_{CD3} comprises one or more light chain framework sequence (i.e. the FR1, FR2, FR3 and/or FR4 sequence) of the light chain variable region sequence of SEQ ID NO: 14.

In one aspect, the VL_{CD3} comprises an amino acid sequence having at least 95%, at least 96%, at least 97%, at least 98% or at least 99% sequence identity to the VL sequence of SEQ ID NO: 14. In one aspect, the VL_{CD3} comprises an amino acid sequence having at least 95% sequence identity to the VL sequence of SEQ ID NO: 14. In one aspect, the VL_{CD3} comprises an amino acid sequence having at least 98% sequence identity to the VL sequence of SEQ ID NO: 14.

In one aspect, a VL sequence having at least 95%, 96%, 97%, 98%, or 99% identity contains substitutions (e.g., conservative substitutions), insertions, or deletions relative to the reference sequence, but an antibody (or antigen binding domain) comprising that sequence retains the ability to bind to CD3. In one aspect, a total of 1 to 10 amino acids have been substituted, inserted and/or deleted in the amino acid sequence of SEQ ID NO: 14. In one aspect, substitutions, insertions, or deletions occur in regions outside the CDRs (i.e., in the FRs).

In one aspect, the VL_{CD3} comprises the amino acid sequence of SEQ ID NO: 14 comprising up to two (i.e. 0, 1 or 2) amino acid substitutions within said amino acid sequence. In one aspect, the VL_{CD3} comprises the amino acid sequence of SEQ ID NO: 14 comprising one amino acid substitution within said amino acid sequence. In one aspect, the VL_{CD3} comprises the amino acid sequence of SEQ ID NO: 14 comprising two amino acid substitutions within said amino acid sequence. In one aspect, the substitutions are in the FR of the VL.

In one aspect, the VL_{CD3} comprises the LCDR 1 of SEQ ID NO: 5, the LCDR 2 of SEQ ID NO: 6, and the LCDR 3 of SEQ ID NO: 13, and an amino acid sequence having at least 95%, at least 96%, at least 97%, at least 98% or at least 99% sequence identity to the amino acid sequence of SEQ ID NO: 14. In one aspect, the VL_{CD3} comprises the LCDR 1 of SEQ ID NO: 5, the LCDR 2 of SEQ ID NO: 6, and the LCDR 3 of SEQ ID NO: 13, and an amino acid sequence having at least 95% sequence identity to the amino acid sequence of SEQ ID NO: 14. In one aspect, the VL_{CD3} comprises the LCDR 1 of SEQ ID NO: 5, the LCDR 2 of SEQ ID NO: 6, and the LCDR 3 of SEQ ID NO: 13, and an amino acid sequence having at least 98% sequence identity to the amino acid sequence of SEQ ID NO: 14.

In one aspect, the VL_{CD3} comprises the LCDR 1 of SEQ ID NO: 5, the LCDR 2 of SEQ ID NO: 6, and the LCDR 3 of SEQ ID NO: 13, and the amino acid sequence of SEQ ID NO: 14 comprising up to two (i.e. 0, 1 or 2) amino acid substitutions within (the framework region of) said amino acid sequence. In one aspect, the VL_{CD3} comprises the LCDR 1 of SEQ ID NO: 5, the LCDR 2 of SEQ ID NO: 6, and the LCDR 3 of SEQ ID NO: 13, and the amino acid sequence of SEQ ID NO: 14 comprising one amino acid substitution within (the framework region of) said amino acid sequence. In one aspect, the VL_{CD3} comprises the LCDR 1 of SEQ ID NO: 5, the LCDR 2 of SEQ ID NO: 6, and the LCDR 3 of SEQ ID NO: 13, and the amino acid sequence of SEQ ID NO: 14 comprising two amino acid substitutions within (the framework region of) said amino acid sequence.

In one aspect, the VL_{CD3} comprises the light chain CDR sequences (i.e. LCDR1, LCDR2 and LCDR3 amino acid sequences) of the VL of SEQ ID NO: 14.

In one aspect, the VL_{CD3} comprises the light chain CDR sequences of the VL of SEQ ID NO: 14, and a framework sequence having at least 95%, at least 96%, at least 97%, at least 98% or at least 99% sequence identity to the framework sequence of the VL of SEQ ID NO: 14. In one aspect, the VL_{CD3} comprises the light chain CDR sequences of the VL of SEQ ID NO: 14, and a framework sequence having at least 95% sequence identity to the framework sequence of the VL of SEQ ID NO: 14. In one aspect, the VL_{CD3} comprises the light chain CDR sequences of the VL of SEQ ID NO: 14, and a framework sequence having at least 98% sequence identity to the framework sequence of the VL of SEQ ID NO: 14.

In one aspect, the VL_{CD3} comprises the light chain CDR sequences of the VL of SEQ ID NO: 14, and the framework sequence of the VH of SEQ ID NO: 14 comprising up to two (i.e. 0, 1 or 2) amino acid substitutions within said framework sequence. In one aspect, the VL_{CD3} comprises the light chain CDR sequences of the VL of SEQ ID NO: 14, and the framework sequence of the VL of SEQ ID NO: 14 comprising one amino acid substitution within said framework sequence. In one aspect, the VL_{CD3} comprises the light chain CDR sequences of the VL of SEQ ID NO: 14, and the framework sequence of the VL of SEQ ID NO: 14 comprising two amino acid substitutions within said framework sequence.

Preferably, the CDR sequences of the VL according to the above aspects are according to the Kabat definition. Alternatively, the CDRs of the VL according to the above aspects are according to the Chothia definition. Further alternatively, the CDRs of the VL according to the above aspects are according to the Contact definition. Still further alternatively, the CDRs of the VL according to the above aspects are according to the IMGT definition.

In one aspect, the VL_{CD3} comprises the amino acid sequence of SEQ ID NO: 14. Optionally, the VL_{CD3} comprises the amino acid sequence of SEQ ID NO: 14, including post-translational modifications of that sequence.

In particular aspects, the antibody (or the first antigen binding domain, as described herein) comprises a VH sequence (VH_{CD3}) as in any of the aspects provided above, and a VL sequence (VL_{CD3}) as in any of the aspects provided above.

In one aspect, the antibody (or the first antigen binding domain, as described herein) comprises a heavy chain variable region (VH_{CD3}) comprising the HCDR 1 of SEQ ID NO: 1, the HCDR 2 of SEQ ID NO: 2, and the HCDR 3 of SEQ ID NO: 10; and a light chain variable region (VL_{CD3}) comprising the LCDR 1 of SEQ ID NO: 5, the LCDR 2 of SEQ ID NO: 6 and the LCDR 3 of SEQ ID NO: 13.

In one aspect, the antibody (or the first antigen binding domain, as described herein) comprises a heavy chain variable region (VH_{CD3}) comprising an amino acid sequence having at least 95%, at least 96%, at least 97%, at least 98% or at least 99% sequence identity to the VH sequence of SEQ ID NO: 12; and a light chain variable region (VL_{CD3}) comprising an amino acid sequence having at least 95%, at least 96%, at least 97%, at least 98% or at least 99% sequence identity to the VL sequence of SEQ ID NO: 14.

In one aspect, the antibody (or the first antigen binding domain, as described herein) comprises a heavy chain variable region (VH_{CD3}) comprising the amino acid sequence of SEQ ID NO: 12 comprising up to two (i.e. 0, 1 or 2) amino acid substitutions within said amino acid sequence; and a light chain variable region (VL_{CD3}) comprising the amino acid sequence of SEQ ID NO: 14 comprising up to two (i.e. 0, 1 or 2) amino acid substitutions within said amino acid sequence.

In one aspect, the antibody (or the first antigen binding domain, as described herein) comprises a heavy chain variable region (VH_{CD3}) comprising the HCDR 1 of SEQ ID NO: 1, the HCDR 2 of SEQ ID NO: 2, and the HCDR 3 of SEQ ID NO: 10, and an amino acid sequence having at least 95%, at least 96%, at least 97%, at least 98% or at least 99% sequence identity to the amino acid sequence of SEQ ID NO: 12; and a light chain variable region (VL_{CD3}) comprising the LCDR 1 of SEQ ID NO: 5, the LCDR 2 of SEQ ID NO: 6, and the LCDR 3 of SEQ ID NO: 13, and an amino acid sequence having at least 95%, at least 96%, at least 97%, at least 98% or at least 99% sequence identity to the amino acid sequence of SEQ ID NO: 14.

In one aspect, the antibody (or the first antigen binding domain, as described herein) comprises a heavy chain variable region (VH_{CD3}) comprising the HCDR 1 of SEQ ID NO: 1, the HCDR 2 of SEQ ID NO: 2, and the HCDR 3 of SEQ ID NO: 10, and the amino acid sequence of SEQ ID NO: 12 comprising up to two (i.e. 0, 1 or 2) amino acid substitutions within (the framework region of) said amino acid sequence; and a light chain variable region (VL_{CD3}) comprising the LCDR 1 of SEQ ID NO: 5, the LCDR 2 of SEQ ID NO: 6, and the LCDR 3 of SEQ ID NO: 13, and the amino acid sequence of SEQ ID NO: 14 comprising up to two (i.e. 0, 1 or 2) amino acid substitutions within (the framework region of) said amino acid sequence.

In one aspect, the antibody (or the first antigen binding domain, as described herein) comprises a heavy chain variable region (VH_{CD3}) comprising the heavy chain CDR sequences (i.e. HCDR1, HCDR2 and HCDR3 amino acid sequences) of the VH of SEQ ID NO: 12; and a light chain variable region (VL_{CD3}) comprising the light chain CDR sequences (i.e. LCDR1, LCDR2 and LCDR3 amino acid sequences) of the VL of SEQ ID NO: 14.

In one aspect, the antibody (or the first antigen binding domain, as described herein) comprises a heavy chain variable region (VH_{CD3}) comprising the heavy chain CDR sequences of the VH of SEQ ID NO: 12, and a framework sequence having at least 95%, at least 96%, at least 97%, at least 98% or at least 99% sequence identity to the framework sequence of the VH of SEQ ID NO: 12; and a light chain variable region (VL_{CD3}) comprising the light chain CDR sequences of the VL of SEQ ID NO: 14, and a framework sequence having at least 95%, at least 96%, at least 97%, at least 98% or at least 99% sequence identity to the framework sequence of the VL of SEQ ID NO: 14.

In one aspect, the antibody (or the first antigen binding domain, as described herein) comprises a heavy chain variable region (VH_{CD3}) comprising the heavy chain CDR sequences of the VH of SEQ ID NO: 12, and the framework sequence of the VH of SEQ ID NO: 12 comprising up to two (i.e. 0, 1 or 2) amino acid substitutions within said framework sequence; and a light chain variable region (VL_{CD3}) comprising the light chain CDR sequences of the VL of SEQ ID NO: 14, and the framework sequence of the VL of SEQ ID NO: 14 comprising up to two (i.e. 0, 1 or 2) amino acid substitutions within said framework sequence.

Preferably, the CDR sequences of the VH and/or VL according to the above aspects are according to the Kabat definition. Alternatively, the CDRs of the VH and/or VL according to the above aspects are according to the Chothia definition. Further alternatively, the CDRs of the VH and/or VL according to the above aspects are according to the Contact definition. Still further alternatively, the CDRs of the VH and/or VL according to the above aspects are according to the IMGT definition.

In one aspect, the antibody (or the first antigen binding domain, as described herein) comprises a heavy chain variable region (VH_{CD3}) comprising the amino acid sequence of SEQ ID NO: 12; and a light chain variable region (VL_{CD3}) comprising the amino acid sequence of SEQ ID NO: 14.

Further aspects in relation to any of the antibodies described above are provided in the following.

In one aspect, the antibody is an isolated antibody and/or a purified antibody.

In one aspect, the antibody is a monoclonal antibody.

In one aspect, the antibody is an IgG, particularly an IgG₁, antibody. In one aspect, the antibody is a full-length antibody, e.g. a full-length IgG, particularly IgG₁, antibody or other antibody class or isotype as defined herein.

In one aspect, the antibody comprises an Fc domain, particularly an IgG Fc domain, more particularly an IgG₁ Fc domain. In one aspect, the Fc domain is a human Fc domain. In one aspect, the Fc domain is a human IgG₁ Fc domain. The Fc domain is composed of a first and a second subunit and may incorporate any of the features, singly or in combination, described in hereinbelow in relation to Fc domains, including Fc domain variants.

In one aspect, the antibody comprises a human constant region. In one aspect, the antibody is an immunoglobulin molecule comprising a human constant region, particularly an IgG class immunoglobulin molecule comprising a human CH1, CH2, CH3 and/or CL domain. Exemplary sequences of human constant domains are given in SEQ ID NOs 34 and 35 (human kappa and lambda CL domains, respectively) and SEQ ID NO: 36 (human IgG₁ heavy chain constant domains CH1-CH2-CH3). In one aspect, the antibody comprises a light chain constant region comprising an amino acid sequence that is at least about 95%, 96%, 97%, 98%, 99% or 100% identical to the amino acid sequence of SEQ ID NO: 34 or SEQ ID NO: 35, particularly the amino acid sequence of SEQ ID NO: 34. In one aspect, the antibody comprises a heavy chain constant region comprising an amino acid sequence that is at least about 95%, 96%, 97%, 98%, 99% or 100% identical to the amino acid sequence of SEQ ID NO: 36. Particularly, the heavy chain constant region may comprise amino acid mutations in the Fc domain as described herein.

### 3. Antibody Fragments

In one aspect, an antibody provided herein is an antibody fragment.

In one aspect, the antibody fragment is a Fab, Fab′, Fab′-SH, or F(ab′)₂ molecule, in particular a Fab molecule as described herein. “Fab′ molecule” differ from Fab molecules by the addition of residues at the carboxy terminus of the CH1 domain including one or more cysteines from the antibody hinge region. Fab′-SH are Fab′ molecules in which the cysteine residue(s) of the constant domains bear a free thiol group. Pepsin treatment yields an F(ab′)₂ molecule that has two antigen-binding sites (two Fab molecules) and a part of the Fc domain.

In another aspect, the antibody fragment is a diabody, a triabody or a tetrabody. “Diabodies” are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

In a further aspect, the antibody fragment is a single chain Fab molecule. A “single chain Fab molecule” or “scFab” is a polypeptide consisting of an antibody heavy chain variable domain (VH), an antibody heavy chain constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL. In particular, said linker is a polypeptide of at least 30 amino acids, preferably between 32 and 50 amino acids. Said single chain Fab molecules are stabilized via the natural disulfide bond between the CL domain and the CH1 domain. In addition, these single chain Fab molecules might be further stabilized by generation of interchain disulfide bonds via insertion of cysteine residues (e.g., position 44 in the variable heavy chain and position 100 in the variable light chain according to Kabat numbering).

In another aspect, the antibody fragment is a single-chain variable fragment (scFv). A “single-chain variable fragment” or “scFv” is a fusion protein of the variable domains of the heavy (VH) and light chains (VL) of an antibody, connected by a linker. In particular, the linker is a short polypeptide of 10 to 25 amino acids and is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458.

In one aspect, the antibody is an antibody fragment selected from the group of an Fv molecule, a scFv molecule, a Fab molecule, and a F(ab′)₂ molecule; particularly a Fab molecule.

Antibody fragments can be made by various techniques, including but not limited to proteolytic digestion of a full-length antibody as well as recombinant production by recombinant host cells (e.g., E. coli), as described herein.

### 4. Humanized Antibodies

In one aspect, an antibody provided herein is a humanized antibody. Typically, a non-human antibody is humanized to reduce immunogenicity to humans, while retaining the specificity and affinity of the parental non-human antibody. Generally, a humanized antibody comprises one or more variable domains in which the CDRs (or portions thereof) are derived from a non-human antibody, and FRs (or portions thereof) are derived from human antibody sequences. A humanized antibody optionally will also comprise at least a portion of a human constant region. In one aspect, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 5. Glycosylation Variants

In one aspect, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc domain, the oligosaccharide attached thereto may be altered. Native antibodies produced by mammalian cells typically comprise a branched, biantennary oligosaccharide that is generally attached by an N-linkage to Asn297 of the CH2 domain of the Fc domain. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody of the invention may be made in order to create antibody variants with certain improved properties.

In one aspect, antibody variants are provided having a non-fucosylated oligosaccharide, i.e. an oligosaccharide structure that lacks fucose attached (directly or indirectly) to an Fc domain. Such non-fucosylated oligosaccharide (also referred to as “afucosylated” oligosaccharide) particularly is an N-linked oligosaccharide which lacks a fucose residue attached to the first GlcNAc in the stem of the biantennary oligosaccharide structure. In one aspect, antibody variants are provided having an increased proportion of non-fucosylated oligosaccharides in the Fc domain as compared to a native or parent antibody. For example, the proportion of non-fucosylated oligosaccharides may be at least about 20%, at least about 40%, at least about 60%, at least about 80%, or even about 100% (i.e. no fucosylated oligosaccharides are present). The percentage of non-fucosylated oligosaccharides is the (average) amount of oligosaccharides lacking fucose residues, relative to the sum of all oligosaccharides attached to Asn 297 (e. g. complex, hybrid and high mannose structures) as measured by MALDI-TOF mass spectrometry, as described in WO 2006/082515, for example. Asn297 refers to the asparagine residue located at about position 297 in the Fc domain (EU numbering of Fc domain residues); however, Asn297 may also be located about ±3 amino acids upstream or downstream of position 297, i.e., between positions 294 and 300, due to minor sequence variations in antibodies. Such antibodies having an increased proportion of non-fucosylated oligosaccharides in the Fc domain may have improved FcγRIIIa receptor binding and/or improved effector function, in particular improved ADCC function. See, e.g., US 2003/0157108; US 2004/0093621.

Examples of cell lines capable of producing antibodies with reduced fucosylation include Lec13 CHO cells deficient in protein fucosylation (Ripka et al. Arch. Biochem. Biophys. 249:533-545 (1986); US 2003/0157108; and WO 2004/056312, especially at Example 11), and knockout cell lines, such as alpha-1,6-fucosyltransferase gene, FUT8, knockout CHO cells (see, e.g., Yamane-Ohnuki et al. Biotech. Bioeng. 87:614-622 (2004); Kanda, Y. et al., Biotechnol. Bioeng., 94(4):680-688 (2006); and WO 2003/085107), or cells with reduced or abolished activity of a GDP-fucose synthesis or transporter protein (see, e.g., US2004259150, US2005031613, US2004132140, US2004110282).

In a further aspect, antibody variants are provided with bisected oligosaccharides, e.g., in which a biantennary oligosaccharide attached to the Fc domain of the antibody is bisected by GlcNAc. Such antibody variants may have reduced fucosylation and/or improved ADCC function as described above.

Examples of such antibody variants are described, e.g., in Umana et al., Nat Biotechnol 17, 176-180 (1999); Ferrara et al., Biotechn Bioeng 93, 851-861 (2006); WO 99/54342; WO 2004/065540, WO 2003/011878.

Antibody variants with at least one galactose residue in the oligosaccharide attached to the Fc domain are also provided. Such antibody variants may have improved CDC function. Such antibody variants are described, e.g., in WO 1997/30087; WO 1998/58964; and WO 1999/22764.

### 6. Antibody Derivatives

In one aspect, an antibody provided herein may be further modified to contain additional non-proteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### 7. Fc Domain Variants

In one aspect, one or more amino acid modifications may be introduced into the Fc domain of an antibody provided herein, thereby generating an Fc domain variant. The Fc domain variant may comprise a human Fc domain sequence (e.g., a human IgG₁, IgG₂, IgG₃ or IgG₄ Fc domain sequence) comprising an amino acid modification (e.g., a substitution) at one or more amino acid positions. Fc domain variants that may be applied to the antibody provided herein are also described in section 6 hereinbelow in relation to multispecific antibodies.

In one aspect, an antibody provided herein comprises an Fc domain with one or more amino acid substitution(s) which increase binding to human FcγR (e.g. FcγRIIIa) and/or effector function (e.g. ADCC), e.g., substitutions at positions 239, 298, 330, 332, 333 and/or 334 of the Fc domain (Kabat EU numbering of residues). In one aspect, the substitutions are S298A, E333A and K334A in an Fc domain derived from a human IgG₁ Fc domain (see e.g. Shields et al. (2001) J. Biol. Chem. 276, 6591-6604). In one aspect, the substitutions are S239D, 1332E and optionally A330L in an Fc domain derived from a human IgG₁ Fc domain (see e.g Lazar et al. (2006) Proc. Natl. Acad. Sci. U.S.A. 103, 4005-4010).

In one aspect, an antibody provided herein comprises an Fc domain with one or more amino acid substitution(s) which reduce binding to human FcγR (e.g. FcγRIIIa) and/or effector function (e.g. ADCC), e.g., substitutions at positions 228, 233, 234, 235, 265, 267, 297, 329 and/or 331 of the Fc domain (Kabat EU numbering of residues).

In one aspect, the substitutions are L234A and L235A (LALA) in an Fc domain derived from a human IgG₁ Fc domain. In one aspect, the Fc domain further comprises a D265A and/or P329G substitution. In one aspect, the substitutions are L234A, L235A and P329G (LALA-PG) in an Fc domain derived from a human IgG₁ Fc domain. See, e.g., WO 2012/130831, Schlothauer et al., Protein Eng Des Sel 29, 457-466 (2016). In another aspect, the substitutions are L234A, L235A and D265A (LALA-DA) in an Fc domain derived from a human IgG₁ Fc domain.

In one aspect, the substitutions are S228P and L235E (SPLE) in an Fc domain derived from a human IgG₄ Fc domain. In one aspect, the Fc domain further comprises a P329G substitution. In one aspect, the substitutions are S228P, L235E and P329G (SPLE-PG) in an Fc domain derived from a human IgG4 Fc domain. See, e.g., WO 2012/130831, Schlothauer et al., Protein Eng Des Sel 29, 457-466 (2016).

In one aspect, the substitution is N297A (NA), N297G (NG) or N297Q (NQ) in an Fc domain derived from a human IgG₁ Fc domain. In one aspect, the Fc domain further comprises a D265A substitution. In one aspect, the substitutions are D265A and N297A (DANA), or D265A and N297G (DANG) in an Fc domain derived from a human IgG₁ Fc domain.

In one aspect, the substitutions are E233P, L234V, L235A and G236del in an Fc domain derived from a human IgG₁ Fc domain (see e.g. Armour et al., Eur. J. Immunol. 29, 2613-2624 (1999)). In one aspect, the Fc domain further comprises a N297G or S267 substitution.

In one aspect, the substitutions are L234F, L235E and D265A (FEA), or L234F, L235E and P331S (FES), in an Fc domain derived from a human IgG₁ Fc domain.

In one aspect, an antibody provided herein comprises an Fc domain with one or more amino acid substitution(s) which decrease binding to human FcRn and/or serum half-life of the antibody, e.g. substitutions at positions 253, 310 and/or 435 (Kabat EU numbering of residues). In one aspect, the substitutions are 1253A, H310A and H435A (AAA) in an Fc domain derived from a human IgG₁ Fc domain.

In one aspect, an antibody provided herein comprises an Fc domain with one or more amino acid substitution(s) which increase binding to human FcRn and/or serum half-life of the antibody, e.g. substitutions at positions 252, 254, 256, 428 and/or 434 (Kabat EU numbering of residues). In one aspect, the substitutions are M252Y, S254T and T256E in an Fc domain derived from a human IgG₁ Fc domain (see, e.g., Dall'Acqua et al. J Biol Chem 281, 23514-23524 (2006); WO 2002/60919). In one aspect, the substitutions are M428L and N434S (see e.g. Zalevsky et al. Nat Biotech 28, 157-159 (2010); WO 2009/086320). In one aspect, the substitutions are M428L and N434A.

In one aspect, an antibody provided herein comprises an Fc domain with one or more amino acid substitution(s) which reduce binding to rheumatoid factor, e.g. substitutions at positions 424, 436, 438 and/or 440 (Kabat EU numbering of residues). In one aspect, the substitutions are Q438R and S440E (RE) (see e.g. Maeda et al. MABS 9, 844-853 (2017)).

In one aspect, amino acid substitution(s) which increase FcRn binding are combined with amino acid substitution(s) which reduce binding to rheumatoid factor, as described e.g. in Maeda et al. MABS 9, 844-853 (2017). In one aspect, the substitutions are M428L, N434A and Y436T, or M428L, N434S and Y436T, in an Fc domain derived from a human IgG₁ Fc domain. In one aspect, the substitutions are N434A, Q438R, S440E, and optionally Y436T or Y436V, in an Fc domain derived from a human IgG₁ Fc domain. In one aspect, the substitutions are M428L, N434A, Q438R, S440E, and optionally Y436T or Y436V, in an Fc domain derived from a human IgG₁ Fc domain.

In one aspect, an antibody provided herein comprises an Fc domain with one or more amino acid substitution(s) which increase the antibody's isoelectric point (pl), e.g. substitutions at positions 311 and/or 434 (Kabat EU numbering of residues). In one aspect, the substitutions are Q311R and P343R.

In one aspect, an antibody provided herein comprises an Fc domain with one or more amino acid substitution(s) which increase affinity to human FcγRIIb, e.g. substitutions at positions 234, 235, 236, 238, 250, 264, 268, 295, 307, 326 and/or 330 (Kabat EU numbering of residues). In one aspect, the substitutions are L235W, G236N, H268D, Q295L, K326T and A330K, or L234Y, P238D, T250V, V2641, T307P and A330K.

For additional Fc domain mutations see e.g. Abdeldaim and Schindowski, Pharmaceutics 15(10): 2402 (2023).

The C-terminus of the Fc domain of an antibody provided herein may be a complete C-terminus ending with the amino acid residues PGK. The C-terminus of the Fc domain may also be a shortened C-terminus in which one or two of the C terminal amino acid residues have been removed. In one aspect, the C-terminus of the Fc domain is a shortened C-terminus ending with the amino acid residue P. In one aspect, the C-terminus of the Fc domain is a shortened C-terminus ending with the amino acid residues PG. In one aspect, an antibody comprising an Fc domain as specified herein, comprises the C-terminal glycine-lysine dipeptide (G446 and K447, Kabat EU numbering of amino acid positions). In one aspect, an antibody comprising an Fc domain as specified herein, comprises a C-terminal glycine residue (G446, Kabat EU numbering of amino acid positions).

### 8. Multispecific Antibodies

In one aspect, the antibody is a multispecific antibody, particularly a bispecific antibody.

Techniques for making multispecific antibodies include, but are not limited to, recombinant co-expression of two immunoglobulin heavy chain-light chain pairs having different specificities (see Milstein and Cuello, Nature 305: 537 (1983)) and “knob-in-hole” engineering (see, e.g., Carter et al., J Immunol Meth 248, 7-15 (2001)). Multispecific antibodies may also be made by engineering electrostatic steering effects for making antibody Fc-heterodimeric molecules (see, e.g., WO 2009/089004); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992) and WO 2011/034605); using the common light chain technology for circumventing the light chain mis-pairing problem (see, e.g., WO 98/50431); using “diabody” technology for making bispecific antibody fragments (see, e.g., Hollinger et al., Proc.

Natl. Acad. Sci. USA, 90:6444-6448 (1993)); and using single-chain Fv (sFv) dimers (see, e.g., Gruber et al., J. Immunol., 152:5368 (1994)).

Engineered antibodies with three or more antigen binding sites, including for example, “Octopus antibodies”, or DVD-Ig are also included herein (see, e.g., WO 2001/77342 and WO 2008/024715). Other examples of multispecific antibodies with three or more antigen binding sites can be found in WO 2010/115589, WO 2010/112193, WO 2010/136172, WO 2010/145792, and WO 2013/026831. The multispecific antibody may also include a “Dual Acting FAb” or “DAF” comprising an antigen binding site that binds to two different antigens, or two different epitopes of the same antigen (see, e.g., US 2008/0069820 and WO 2015/095539). Multispecific antibodies also include a “DutaFab” wherein a single pair of a VH domain and a VL domain may bind to two different epitopes and wherein one paratope comprises amino acid residues from HCDR2, LCDR1 and LCDR3 and the other paratope comprises amino acid residues from HCDR1, HCDR3 and LCDR2 (see, e.g., WO 2012/163520).

Multispecific antibodies may also be provided in an asymmetric form with a domain crossover in one or more binding arms of the same antigen specificity, i.e. by exchanging the VH/VL domains (see e.g., WO 2009/080252 and WO 2015/150447), the CH1/CL domains (see e.g., WO 2009/080253) or the complete Fab arms (see e.g., WO 2009/080251, WO 2016/016299). Also see Schaefer et al, PNAS, 108 (2011) 1187-1191, and Klein at al., MAbs 8 (2016) 1010-20.

Asymmetrical Fab arms can also be engineered by introducing charged or non-charged amino acid mutations into domain interfaces to direct correct Fab (i.e. heavy and light chain) pairing.

Exemplary Fab pairing amino acid mutations are Q39E (Kabat numbering) and S183K (Kabat EU numbering) in the heavy chain and Q38K (Kabat numbering) and V133E (Kabat EU numbering) in the light chain, or Q39K (Kabat numbering) and S183E (Kabat EU numbering) in the heavy chain and Q38E (Kabat numbering) and V133K (Kabat EU numbering) in the light chain (see e.g. WO 2016/172485).

Further Fab pairing mutations include 124K, 124R or 124H (Kabat numbering) and 123K, 123R or 123H (Kabat numbering) in the light chain and 147E or 147D (Kabat EU numbering) and 213E or 213D (Kabat EU numbering) in the heavy chain (see e.g. WO 2015/150447).

To promote the correct association of heavy chains in asymmetric multispecific antibodies, their heavy chains may be engineered to comprise e.g. sterically (“knob-in-hole”) or electrostatically complementary amino acid mutations, salt bridges, and/or disulfide bonds. The knob-in-hole technology is described e.g. in U.S. Pat. Nos. 5,731,168; 7,695,936; Ridgway et al., Prot Eng 9, 617-621 (1996), Atwell et al., J. Mol. Biol. 270, 26 (1997), Merchant et al., Nat Biotechnol 16, 677-681 (1998), and Carter, J Immunol Meth 248, 7-15 (2001). According to the knob-in-hole technology, a bispecific antibody comprising a human IgG₁ Fc domain may comprise the“knob” mutation T366W on the first heavy chain, and “hole” mutations Y407V and optionally T366S and L368A (all Kabat EU numbering) on the second heavy chain (T366W/T366S:L368A:Y407V). Additionally, the antibody may comprise a S354C substitution on the first heavy chain and a Y349C substitution (both Kabat EU numbering) on the second heavy chain, forming a disulfide bond (T366W:S354C/Y349C:T366S:L368A:Y407V).

Further examples of amino acid mutations (e.g. substitutions) that may be comprised in multispecific (e.g. bispecific) antibodies include the substitution S228P (Kabat EU numbering) in antibodies comprising an IgG₄ Fc domain, e.g. for preventing Fab arm exchange (see Stubenrauch et al., Drug Metabolism and Disposition 38, 84-91 (2010)).

Various further molecular formats for multispecific antibodies are known in the art and are included herein (see e.g., Spiess et al., Mol Immunol 67: 95-106 (2015)).

A particular type of multispecific antibodies are bispecific antibodies designed to simultaneously bind to a surface antigen on a target cell, e.g., a tumor cell, and to an activating, invariant component of the T cell receptor (TCR) complex, such as CD3, for retargeting of T cells to kill target cells. Examples of bispecific antibody formats that may be useful for this purpose include, but are not limited to, the so-called “BiTE” (bispecific T cell engager) molecules wherein two scFv molecules are fused by a flexible linker (see, e.g., WO 2004/106381, WO 2005/061547, WO 2007/042261, and WO 2008/119567, Nagorsen and Bauerle, Exp Cell Res 317, 1255-1260 (2011)); diabodies (Holliger et al., Prot Eng 9, 299-305 (1996)) and derivatives thereof, such as tandem diabodies (“TandAb”; Kipriyanov et al., J Mol Biol 293, 41-56 (1999)); “DART” (dual affinity retargeting) molecules which are based on the diabody format but feature a C-terminal disulfide bridge for additional stabilization (Johnson et al., J Mol Biol 399, 436-449 (2010)), and so-called triomabs, which are full-length hybrid mouse/rat IgG molecules (reviewed in Seimetz et al., Cancer Treat Rev 36, 458-467 (2010)). Particular T cell bispecific antibody formats included herein are described in WO 2013/026833, WO 2013/026839, WO 2016/020309; Bacac et al., Oncoimmunology 5(8) (2016) e1203498.

Details of the (multispecific) antibody of the present invention are further described in the following.

In one aspect, the antibody is a multispecific antibody and comprises
(a) a first antigen binding domain that binds to CD3, comprising a VH_{CD3} and a VL_{CD3} as described hereinabove, and
(b) a second and optionally a third antigen binding domain that bind to a second antigen.

According to preferred aspects of the invention, the antigen binding domains comprised in the (multispecific) antibody are Fab molecules (i.e. antigen binding domains composed of a heavy and a light chain, each comprising a variable and a constant domain). In one aspect, the first, the second and/or, where present, the third antigen binding domain is a Fab molecule. In one aspect, said Fab molecule is human. In a preferred aspect, said Fab molecule is humanized. In another preferred aspect, said Fab molecule comprises human heavy and light chain constant domains.

Preferably, at least one of the antigen binding domains is a crossover Fab molecule. Such modification reduces mispairing of heavy and light chains from different Fab molecules, thereby improving the yield and purity of the (multispecific) antibody in recombinant production. In a preferred crossover Fab molecule useful for the (multispecific) antibody of the invention, the variable domains of the Fab light chain and the Fab heavy chain (VL and VH, respectively) are exchanged. Even with this domain exchange, however, the preparation of the (multispecific) antibody may comprise certain side products due to a so-called Bence Jones-type interaction between mispaired heavy and light chains (see Schaefer et al, PNAS, 108 (2011) 11187-11191). To further reduce mispairing of heavy and light chains from different Fab molecules and thus increase the purity and yield of the desired (multispecific) antibody, charged amino acids with opposite charges may be introduced at specific amino acid positions in the CH1 and CL domains of either the Fab molecule binding to CD3, or the Fab molecule(s) binding to the second antigen (e.g. a target cell antigen such as CD20), as further described herein. Charge modifications are made either in the conventional Fab molecule(s) comprised in the (multispecific) antibody, or in the VH/VL crossover Fab molecule(s) comprised in the (multispecific) antibody (but not in both). In preferred aspects, the charge modifications are made in the conventional Fab molecule(s) comprised in the (multispecific) antibody (which in preferred aspects bind(s) to the second antigen, e.g. a target cell antigen such as CD20).

In a preferred aspect according to the invention, the (multispecific) antibody is capable of simultaneous binding to the first antigen (i.e. CD3), and the second antigen (e.g. a target cell antigen such as CD20). In one aspect, the (multispecific) antibody is capable of crosslinking a T cell and a target cell by simultaneous binding to CD3 and a target cell antigen. In an even more preferred aspect, such simultaneous binding results in lysis of the target cell, particularly a target cell antigen (e.g. CD20)-expressing tumor cell. In one aspect, such simultaneous binding results in activation of the T cell. In other aspects, such simultaneous binding results in a cellular response of a T lymphocyte, particularly a cytotoxic T lymphocyte, selected from the group of: proliferation, differentiation, cytokine secretion, cytotoxic effector molecule release, cytotoxic activity, and expression of activation markers. In one aspect, binding of the (multispecific) antibody to CD3 without simultaneous binding to the target cell antigen does not result in T cell activation.

In one aspect, the (multispecific) antibody is capable of re-directing cytotoxic activity of a T cell to a target cell. In a preferred aspect, said re-direction is independent of MHC-mediated peptide antigen presentation by the target cell and and/or specificity of the T cell.

Preferably, a T cell according to any of the aspects of the invention is a cytotoxic T cell. In some aspects the T cell is a CD4⁺ or a CD8⁺ T cell, particularly a CD8⁺ T cell.

In a further aspect, the (multispecific) antibody according to any of the above aspects may incorporate any of the features, singly or in combination, as described in sections 1.-5. below.

### a) First Antigen Binding Domain

The first antigen binding domain of the (multispecific) antibody according to the invention binds to CD3, specifically human CD3, and comprises a heavy chain variable region (VH_{CD3}) and a light chain variable region (VH_{CD3}) as described hereinabove.

In a preferred aspect, the (multispecific) antibody comprises not more than one antigen binding domain that binds to CD3. In one aspect, the (multispecific) antibody provides monovalent binding to CD3.

In one aspect, the first antigen binding domain is an antibody fragment selected from the group of an Fv molecule, a scFv molecule and a Fab molecule. In a particular aspect, the first antigen binding domain is a Fab molecule.

In a preferred aspect, the first antigen binding domain is a crossover Fab molecule as described herein, i.e. a Fab molecule wherein the variable domains VH and VL or the constant domains CH1 and CL of the Fab heavy and light chains are exchanged/replaced by each other. In such aspect, the second and, where present, the third antigen binding domain is preferably a conventional Fab molecule. In aspects where there is more than one antigen binding domain, particularly Fab molecule, that binds to a second antigen comprised in the (multispecific) antibody, the antigen binding domain that binds to CD3 (i.e. the first antigen binding domain) preferably is a crossover Fab molecule and the antigen binding domain that bind to the second antigen (i.e. the second and the third antigen binding domains) are conventional Fab molecules.

In an alternative aspect, the first antigen binding domain is a conventional Fab molecule. In such aspect, the second and, where present, the third antigen binding domain is a crossover Fab molecule as described herein, i.e. a Fab molecule wherein the variable domains VH and VL or the constant domains CH1 and CL of the Fab heavy and light chains are exchanged/replaced by each other. In aspects where there is more than one antigen binding domain, particularly Fab molecule, that binds to CD3 comprised in the (multispecific) antibody, the antigen binding domain that binds to the second antigen preferably is a crossover Fab molecule and the antigen binding domains that bind to CD3 are conventional Fab molecules.

In a preferred aspect, the first antigen binding domain is a Fab molecule wherein the variable domains VL and VH or the constant domains CL and CH1, particularly the variable domains VL and VH, of the Fab light chain and the Fab heavy chain are replaced by each other (i.e. according to such aspect, the first antigen binding domain is a crossover Fab molecule wherein the variable or constant domains of the Fab light chain and the Fab heavy chain are exchanged). In one such aspect, the second and, where present, the third antigen binding domain is a conventional Fab molecule. In one such aspect, not more than one antigen binding domain that binds to CD3 is present in the (multispecific) antibody (i.e. the antibody provides monovalent binding to CD3).

In one aspect, the first antigen binding domain comprises a human constant region. In one aspect, the first antigen binding domain is a Fab molecule comprising a human constant region, particularly a human CH1 and/or CL domain. In one aspect, the first antigen binding domain comprises a light chain constant region comprising an amino acid sequence that is at least 95%, at least 96%, at least 97%, at least 98%, at least 99% or 100% identical to the amino acid sequence of SEQ ID NO: 34.

Particularly, in such aspect, the light chain constant region may comprise amino acid mutations as described herein under “charge modifications” and/or may comprise deletions or substitutions of one or more (particularly two)N-terminal amino acids if in a crossover Fab molecule. In one aspect, the first antigen binding domain comprises a heavy chain constant region comprising an amino acid sequence that is at least 95%, at least 96%, at least 97%, at least 98%, at least 99% or 100% identical to the CH1 domain sequence comprised in the amino acid sequence of SEQ ID NO: 36. Particularly, in such aspect, the heavy chain constant region (specifically CH1 domain) may comprise amino acid mutations as described herein under “charge modifications”.

### b) Second (and Third) Antigen Binding Domain

The second and, where present, the third antigen binding domain of the (multispecific) antibody according to the invention binds to a second antigen.

The second antigen preferably is not CD3, i.e. different from CD3. In one aspect, the second antigen is an antigen expressed on a different cell than CD3 (e.g. expressed on a cell other than a T cell).

In one aspect, the second antigen is a target cell antigen, particularly a tumor cell antigen. In one aspect, the second antigen is a B-cell antigen. In a specific aspect, the second antigen is CD20. The second antigen binding domain is able to direct the (multispecific) antibody to a target site, for example to a specific type of tumor cell that expresses the second antigen.

In one aspect, the second and/or the third antigen binding domain is an antibody fragment selected from the group of an Fv molecule, a scFv molecule, a Fab molecule, and a F(ab′)₂ molecule. In a preferred aspect, the second and/or the third antigen binding domain is a Fab molecule.

In one aspect, the (multispecific) antibody comprises two antigen binding domains, particularly Fab molecules, that bind to the second antigen. In a preferred such aspect, each of these antigen binding domains binds to the same antigenic determinant. In an even more preferred aspect, all of these antigen binding domains are identical, i.e. they have the same molecular format (e.g. conventional or crossover Fab molecule) and comprise the same amino acid sequences including the same amino acid substitutions in the CH1 and CL domain as described herein (if any). In one aspect, the (multispecific) antibody comprises not more than two antigen binding domains, particularly Fab molecules, that bind to the second antigen.

In a preferred aspect, the second and, where present, the third antigen binding domain is a conventional Fab molecule. In such aspect, the first antigen binding domain is a crossover Fab molecule as described herein, i.e. a Fab molecule wherein the variable domains VH and VL or the constant domains CH1 and CL of the Fab heavy and light chains are exchanged/replaced by each other.

In an alternative aspect, the second and, where present, the third antigen binding domain is a crossover Fab molecule as described herein, i.e. a Fab molecule wherein the variable domains VH and VL or the constant domains CH1 and CL of the Fab heavy and light chains are exchanged/replaced by each other. In such aspect, the first antigen binding domain is a conventional Fab molecule.

In one aspect, the second and, where present, the third antigen binding domain comprises a human constant region. In one aspect, the second and, where present, the third antigen binding domain is a Fab molecule comprising a human constant region, particularly a human CH1 and/or CL domain. In one aspect, the second and, where present, the third antigen binding domain comprises a light chain constant region comprising an amino acid sequence that is at least 95%, at least 96%, at least 97%, at least 98%, at least 99% or 100% identical to the amino acid sequence of SEQ ID NO: 34 or SEQ ID NO: 35, particularly the amino acid sequence of SEQ ID NO: 34. Particularly, in such aspect, the light chain constant region may comprise amino acid mutations as described herein under “charge modifications” and/or may comprise deletions or substitutions of one or more (particularly two)N-terminal amino acids if in a crossover Fab molecule. In one aspect, the second and, where present, the third antigen binding domain comprises a heavy chain constant region comprising an amino acid sequence that is at least 95%, at least 96%, at least 97%, at least 98%, at least 99% or 100% identical to the CH1 domain sequence comprised in the amino acid sequence of SEQ ID NO: 36. Particularly, in such aspect, the heavy chain constant region (specifically CH1 domain) may comprise amino acid mutations as described herein under “charge modifications”.

In some aspects, the second antigen is CD20, specifically human CD20.

In one aspect, the second and, where present, the third antigen binding domain comprises a heavy chain variable region (VH_{CD20}) and a light chain variable region (VL_{CD20}).

In one aspect, the VH_{CD20} comprises the heavy chain complementarity determining region (HCDR) 1 of SEQ ID NO: 15, the HCDR 2 of SEQ ID NO: 16, and the HCDR 3 of SEQ ID NO: 17.

In one aspect, the second and, where present, the third antigen binding domain is a humanized antigen binding domain (i.e. an antigen binding domain derived from a humanized antibody). In one aspect, the VH_{CD20} is a humanized variable region. In one aspect, the VH_{CD20} comprises an acceptor human framework, e.g. a human immunoglobulin framework or a human consensus framework.

In some aspects, the VH_{CD20} comprises one or more heavy chain framework sequence (i.e. the FR1, FR2, FR3 and/or FR4 sequence) of the heavy chain variable region sequence of SEQ ID NO: 18.

In one aspect, the VH_{CD20} comprises an amino acid sequence having at least 95%, at least 96%, at least 97%, at least 98% or at least 99% sequence identity to the VH sequence of SEQ ID NO: 18. In one aspect, the VH_{CD20} comprises an amino acid sequence having at least 95% sequence identity to the VH sequence of SEQ ID NO: 18. In one aspect, the VH_{CD20} comprises an amino acid sequence having at least 98% sequence identity to the VH sequence of SEQ ID NO: 18.

In one aspect, a VH sequence having at least 95%, 96%, 97%, 98%, or 99% identity contains substitutions (e.g., conservative substitutions), insertions, or deletions relative to the reference sequence, but an antibody (or antigen binding domain) comprising that sequence retains the ability to bind to CD20. In one aspect, a total of 1 to 10 amino acids have been substituted, inserted and/or deleted in the amino acid sequence of SEQ ID NO: 18. In one aspect, substitutions, insertions, or deletions occur in regions outside the CDRs (i.e., in the FRs).

In one aspect, the VH_{CD20} comprises the amino acid sequence of SEQ ID NO: 18 comprising up to two (i.e. 0, 1 or 2) amino acid substitutions within said amino acid sequence. In one aspect, the VH_{CD20} comprises the amino acid sequence of SEQ ID NO: 18 comprising one amino acid substitution within said amino acid sequence. In one aspect, the VH_{CD20} comprises the amino acid sequence of SEQ ID NO: 18 comprising two amino acid substitutions within said amino acid sequence. In one aspect, the substitutions are in the FR of the VH.

In one aspect, the VH_{CD20} comprises the HCDR 1 of SEQ ID NO: 15, the HCDR 2 of SEQ ID NO: 16, and the HCDR 3 of SEQ ID NO: 17, and an amino acid sequence having at least 95%, at least 96%, at least 97%, at least 98% or at least 99% sequence identity to the amino acid sequence of SEQ ID NO: 18. In one aspect, the VH_{CD20} comprises the HCDR 1 of SEQ ID NO: 15, the HCDR 2 of SEQ ID NO: 16, and the HCDR 3 of SEQ ID NO: 17, and an amino acid sequence having at least 95% sequence identity to the amino acid sequence of SEQ ID NO: 18. In one aspect, the VH_{CD20} comprises the HCDR 1 of SEQ ID NO: 15, the HCDR 2 of SEQ ID NO: 16, and the HCDR 3 of SEQ ID NO: 17, and an amino acid sequence having at least 98% sequence identity to the amino acid sequence of SEQ ID NO: 18.

In one aspect, the VH_{CD20} comprises the HCDR 1 of SEQ ID NO: 15, the HCDR 2 of SEQ ID NO: 16, and the HCDR 3 of SEQ ID NO: 17, and the amino acid sequence of SEQ ID NO: 18 comprising up to two (i.e. 0, 1 or 2) amino acid substitutions within (the framework region of) said amino acid sequence. In one aspect, the VH_{CD20} comprises the HCDR 1 of SEQ ID NO: 15, the HCDR 2 of SEQ ID NO: 16, and the HCDR 3 of SEQ ID NO: 17, and the amino acid sequence of SEQ ID NO: 18 comprising one amino acid substitution within (the framework region of) said amino acid sequence. In one aspect, the VH_{CD20} comprises the HCDR 1 of SEQ ID NO: 15, the HCDR 2 of SEQ ID NO: 16, and the HCDR 3 of SEQ ID NO: 17, and the amino acid sequence of SEQ ID NO: 18 comprising two amino acid substitutions within (the framework region of) said amino acid sequence.

In one aspect, the VH_{CD20} comprises the heavy chain CDR sequences (i.e. HCDR1, HCDR2 and HCDR3 amino acid sequences) of the VH of SEQ ID NO: 18.

In one aspect, the VH_{CD20} comprises the heavy chain CDR sequences of the VH of SEQ ID NO: 18, and a framework sequence having at least 95%, at least 96%, at least 97%, at least 98% or at least 99% sequence identity to the framework sequence of the VH of SEQ ID NO: 18. In one aspect, the VH_{CD20} comprises the heavy chain CDR sequences of the VH of SEQ ID NO: 18, and a framework sequence having at least 95% sequence identity to the framework sequence of the VH of SEQ ID NO: 18. In one aspect, the VH_{CD20} comprises the heavy chain CDR sequences of the VH of SEQ ID NO: 18, and a framework sequence having at least 98% sequence identity to the framework sequence of the VH of SEQ ID NO: 18.

In one aspect, the VH_{CD20} comprises the heavy chain CDR sequences of the VH of SEQ ID NO: 18, and the framework sequence of the VH of SEQ ID NO: 18 comprising up to two (i.e. 0, 1 or 2) amino acid substitutions within said framework sequence. In one aspect, the VH_{CD20} comprises the heavy chain CDR sequences of the VH of SEQ ID NO: 18, and the framework sequence of the VH of SEQ ID NO: 18 comprising one amino acid substitution within said framework sequence. In one aspect, the VH_{CD20} comprises the heavy chain CDR sequences of the VH of SEQ ID NO: 18, and the framework sequence of the VH of SEQ ID NO: 18 comprising two amino acid substitutions within said framework sequence.

Preferably, the CDR sequences of the VH according to the above aspects are according to the Kabat definition. Alternatively, the CDRs of the VH according to the above aspects are according to the Chothia definition. Further alternatively, the CDRs of the VH according to the above aspects are according to the Contact definition. Still further alternatively, the CDRs of the VH according to the above aspects are according to the IMGT definition.

In one aspect, the VH_{CD20} comprises the amino acid sequence of SEQ ID NO: 18. Optionally, the VH_{CD20} comprises the amino acid sequence of SEQ ID NO: 18, including post-translational modifications of that sequence.

In one aspect, the VL_{CD20} comprises the light chain complementarity determining region (LCDR) 1 of SEQ ID NO: 19, the LCDR 2 of SEQ ID NO: 20, and the LCDR 3 of SEQ ID NO: 21.

In one aspect, the second and, where present, the third antigen binding domain is a humanized antigen binding domain (i.e. an antigen binding domain derived from a humanized antibody). In one aspect, the VL_{CD20} is a humanized variable region. In one aspect, the VL_{CD20} comprises an acceptor human framework, e.g. a human immunoglobulin framework or a human consensus framework.

In some aspects, the VL_{CD20} comprises one or more light chain framework sequence (i.e. the FR1, FR2, FR3 and/or FR4 sequence) of the heavy chain variable region sequence of SEQ ID NO: 22.

In one aspect, the VL_{CD20} comprises an amino acid sequence having at least 95%, at least 96%, at least 97%, at least 98% or at least 99% sequence identity to the VL sequence of SEQ ID NO: 22. In one aspect, the VL_{CD20} comprises an amino acid sequence having at least 95% sequence identity to the VL sequence of SEQ ID NO: 22. In one aspect, the VL_{CD20} comprises an amino acid sequence having at least 98% sequence identity to the VL sequence of SEQ ID NO: 22.

In one aspect, a VL sequence having at least 95%, 96%, 97%, 98%, or 99% identity contains substitutions (e.g., conservative substitutions), insertions, or deletions relative to the reference sequence, but an antibody (or antigen binding domain) comprising that sequence retains the ability to bind to CD20. In one aspect, a total of 1 to 10 amino acids have been substituted, inserted and/or deleted in the amino acid sequence of SEQ ID NO: 22. In one aspect, substitutions, insertions, or deletions occur in regions outside the CDRs (i.e., in the FRs).

In one aspect, the VL_{CD20} comprises the amino acid sequence of SEQ ID NO: 22 comprising up to two (i.e. 0, 1 or 2) amino acid substitutions within said amino acid sequence. In one aspect, the VL_{CD20} comprises the amino acid sequence of SEQ ID NO: 22 comprising one amino acid substitution within said amino acid sequence. In one aspect, the VL_{CD20} comprises the amino acid sequence of SEQ ID NO: 22 comprising two amino acid substitutions within said amino acid sequence. In one aspect, the substitutions are in the FR of the VL.

In one aspect, the VL_{CD20} comprises the LCDR 1 of SEQ ID NO: 19, the LCDR 2 of SEQ ID NO: 20, and the LCDR 3 of SEQ ID NO: 21, and an amino acid sequence having at least 95%, at least 96%, at least 97%, at least 98% or at least 99% sequence identity to the amino acid sequence of SEQ ID NO: 22. In one aspect, the VL_{CD}20 comprises the LCDR 1 of SEQ ID NO: 19, the LCDR 2 of SEQ ID NO: 20, and the LCDR 3 of SEQ ID NO: 21, and an amino acid sequence having at least 95% sequence identity to the amino acid sequence of SEQ ID NO: 22. In one aspect, the VL_{CD20} comprises the LCDR 1 of SEQ ID NO: 19, the LCDR 2 of SEQ ID NO: 20, and the LCDR 3 of SEQ ID NO: 21, and an amino acid sequence having at least 98% sequence identity to the amino acid sequence of SEQ ID NO: 22.

In one aspect, the VL_{CD20} comprises the LCDR 1 of SEQ ID NO: 19, the LCDR 2 of SEQ ID NO: 20, and the LCDR 3 of SEQ ID NO: 21, and the amino acid sequence of SEQ ID NO: 22 comprising up to two (i.e. 0, 1 or 2) amino acid substitutions within (the framework region of) said amino acid sequence. In one aspect, the VL_{CD20} comprises the LCDR 1 of SEQ ID NO: 19, the LCDR 2 of SEQ ID NO: 20, and the LCDR 3 of SEQ ID NO: 21, and the amino acid sequence of SEQ ID NO: 22 comprising one amino acid substitution within (the framework region of) said amino acid sequence. In one aspect, the VL_{CD20} comprises the LCDR 1 of SEQ ID NO: 19, the LCDR 2 of SEQ ID NO: 20, and the LCDR 3 of SEQ ID NO: 21, and the amino acid sequence of SEQ ID NO: 22 comprising two amino acid substitutions within (the framework region of) said amino acid sequence.

In one aspect, the VL_{CD20} comprises the light chain CDR sequences (i.e. LCDR1, LCDR2 and LCDR3 amino acid sequences) of the VL of SEQ ID NO: 22.

In one aspect, the VL_{CD20} comprises the light chain CDR sequences of the VL of SEQ ID NO: 22, and a framework sequence having at least 95%, at least 96%, at least 97%, at least 98% or at least 99% sequence identity to the framework sequence of the VL of SEQ ID NO: 22. In one aspect, the VL_{CD20} comprises the light chain CDR sequences of the VL of SEQ ID NO: 22, and a framework sequence having at least 95% sequence identity to the framework sequence of the VL of SEQ ID NO: 22. In one aspect, the VL_{CD20} comprises the light chain CDR sequences of the VL of SEQ ID NO: 22, and a framework sequence having at least 98% sequence identity to the framework sequence of the VL of SEQ ID NO: 22.

In one aspect, the VL_{CD20} comprises the light chain CDR sequences of the VL of SEQ ID NO: 22, and the framework sequence of the VH of SEQ ID NO: 22 comprising up to two (i.e. 0, 1 or 2) amino acid substitutions within said framework sequence. In one aspect, the VL_{CD20} comprises the light chain CDR sequences of the VL of SEQ ID NO: 22, and the framework sequence of the VL of SEQ ID NO: 22 comprising one amino acid substitution within said framework sequence. In one aspect, the VL_{CD20} comprises the light chain CDR sequences of the VL of SEQ ID NO: 22, and the framework sequence of the VL of SEQ ID NO: 22 comprising two amino acid substitutions within said framework sequence.

Preferably, the CDR sequences of the VL according to the above aspects are according to the Kabat definition. Alternatively, the CDRs of the VL according to the above aspects are according to the Chothia definition. Further alternatively, the CDRs of the VL according to the above aspects are according to the Contact definition. Still further alternatively, the CDRs of the VL according to the above aspects are according to the IMGT definition.

In one aspect, the VL_{CD20} comprises the amino acid sequence of SEQ ID NO: 22. Optionally, the VL_{CD20} comprises the amino acid sequence of SEQ ID NO: 22, including post-translational modifications of that sequence.

In particular aspects, the second and, where present, the third antigen binding domain comprises a VH sequence (VH_{CD20}) as in any of the aspects provided above, and a VL sequence (VL_{CD20}) as in any of the aspects provided above.

In one aspect, the second and, where present, the third antigen binding domain comprises a heavy chain variable region (VH_{CD20}) comprising the HCDR 1 of SEQ ID NO: 15, the HCDR 2 of SEQ ID NO: 16, and the HCDR 3 of SEQ ID NO: 17; and a light chain variable region (VL_{CD20}) comprising the LCDR 1 of SEQ ID NO: 19, the LCDR 2 of SEQ ID NO: 20 and the LCDR 3 of SEQ ID NO: 21.

In one aspect, the second and, where present, the third antigen binding domain comprises a heavy chain variable region (VH_{CD20}) comprising an amino acid sequence having at least 95%, at least 96%, at least 97%, at least 98% or at least 99% sequence identity to the VH sequence of SEQ ID NO: 18; and a light chain variable region (VL_{CD20}) comprising an amino acid sequence having at least 95%, at least 96%, at least 97%, at least 98% or at least 99% sequence identity to the VL sequence of SEQ ID NO: 22.

In one aspect, the second and, where present, the third antigen binding domain comprises a heavy chain variable region (VH_{CD20}) comprising the amino acid sequence of SEQ ID NO: 18 comprising up to two (i.e. 0, 1 or 2) amino acid substitutions within said amino acid sequence; and a light chain variable region (VL_{CD20}) comprising the amino acid sequence of SEQ ID NO: 22 comprising up to two (i.e. 0, 1 or 2) amino acid substitutions within said amino acid sequence.

In one aspect, the second and, where present, the third antigen binding domain comprises a heavy chain variable region (VH_{CD20}) comprising the HCDR 1 of SEQ ID NO: 15, the HCDR 2 of SEQ ID NO: 16, and the HCDR 3 of SEQ ID NO: 17, and an amino acid sequence having at least 95%, at least 96%, at least 97%, at least 98% or at least 99% sequence identity to the amino acid sequence of SEQ ID NO: 18; and a light chain variable region (VL_{CD20}) comprising the LCDR 1 of SEQ ID NO: 19, the LCDR 2 of SEQ ID NO: 20, and the LCDR 3 of SEQ ID NO: 21, and an amino acid sequence having at least 95%, at least 96%, at least 97%, at least 98% or at least 99% sequence identity to the amino acid sequence of SEQ ID NO: 22.

In one aspect, the second and, where present, the third antigen binding domain comprises a heavy chain variable region (VH_{CD20}) comprising the HCDR 1 of SEQ ID NO: 15, the HCDR 2 of SEQ ID NO: 16, and the HCDR 3 of SEQ ID NO: 17, and the amino acid sequence of SEQ ID NO: 18 comprising up to two (i.e. 0, 1 or 2) amino acid substitutions within (the framework region of) said amino acid sequence; and a light chain variable region (VL_{CD20}) comprising the LCDR 1 of SEQ ID NO: 19, the LCDR 2 of SEQ ID NO: 20, and the LCDR 3 of SEQ ID NO: 21, and the amino acid sequence of SEQ ID NO: 22 comprising up to two (i.e. 0, 1 or 2) amino acid substitutions within (the framework region of) said amino acid sequence.

In one aspect, the second and, where present, the third antigen binding domain comprises a heavy chain variable region (VH_{CD20}) comprising the heavy chain CDR sequences (i.e. HCDR1, HCDR2 and HCDR3 amino acid sequences) of the VH of SEQ ID NO: 18; and a light chain variable region (VL_{CD20}) comprising the light chain CDR sequences (i.e. LCDR1, LCDR2 and LCDR3 amino acid sequences) of the VL of SEQ ID NO: 22.

In one aspect, the second and, where present, the third antigen binding domain comprises a heavy chain variable region (VH_{CD20}) comprising the heavy chain CDR sequences of the VH of SEQ ID NO: 18, and a framework sequence having at least 95%, at least 96%, at least 97%, at least 98% or at least 99% sequence identity to the framework sequence of the VH of SEQ ID NO: 18; and a light chain variable region (VL_{CD20}) comprising the light chain CDR sequences of the VL of SEQ ID NO: 22, and a framework sequence having at least 95%, at least 96%, at least 97%, at least 98% or at least 99% sequence identity to the framework sequence of the VL of SEQ ID NO: 22.

In one aspect, the second and, where present, the third antigen binding domain comprises a heavy chain variable region (VH_{CD20}) comprising the heavy chain CDR sequences of the VH of SEQ ID NO: 18, and the framework sequence of the VH of SEQ ID NO: 18 comprising up to two (i.e. 0, 1 or 2) amino acid substitutions within said framework sequence; and a light chain variable region (VL_{CD20}) comprising the light chain CDR sequences of the VL of SEQ ID NO: 22, and the framework sequence of the VL of SEQ ID NO: 22 comprising up to two (i.e. 0, 1 or 2) amino acid substitutions within said framework sequence.

Preferably, the CDR sequences of the VH and/or VL according to the above aspects are according to the Kabat definition. Alternatively, the CDRs of the VH and/or VL according to the above aspects are according to the Chothia definition. Further alternatively, the CDRs of the VH and/or VL according to the above aspects are according to the Contact definition. Still further alternatively, the CDRs of the VH and/or VL according to the above aspects are according to the IMGT definition.

In one aspect, the second and, where present, the third antigen binding domain comprises a heavy chain variable region (VH_{CD20}) comprising the amino acid sequence of SEQ ID NO: 18; and a light chain variable region (VL_{CD20}) comprising the amino acid sequence of SEQ ID NO: 22.

### 9. Charge Modifications

The (multispecific) antibody of the invention may comprise amino acid substitutions in Fab molecules comprised therein which are particularly efficient in reducing mispairing of light chains with non-matching heavy chains (Bence-Jones-type side products), which can occur in the production of Fab-based multispecific antibodies with a VH/VL exchange in one (or more, in case of molecules comprising more than two antigen-binding Fab molecules) of their binding arms (see also PCT publication no. WO 2015/150447, particularly the examples therein, incorporated herein by reference in its entirety). The ratio of a desired (multispecific) antibody compared to undesired side products, in particular Bence Jones-type side products occurring in multispecific antibodies with a VH/VL domain exchange in one of their binding arms, can be improved by the introduction of charged amino acids with opposite charges at specific amino acid positions in the CH1 and CL domains (sometimes referred to herein as “charge modifications”).

Accordingly, in some aspects wherein the first and the second and, where present, the third antigen binding domain of the (multispecific) antibody are both Fab molecules, and in one of the antigen binding domains (particularly the first antigen binding domain) the variable domains VL and VH of the Fab light chain and the Fab heavy chain are replaced by each other,
i) in the constant domain CL of the second and, where present, the third antigen binding domain the amino acid at position 124 is substituted by a positively charged amino acid (numbering according to Kabat), and wherein in the constant domain CH1 of the second and, where present, the third antigen binding domain the amino acid at position 147 or the amino acid at position 213 is substituted by a negatively charged amino acid (numbering according to Kabat EU index); or
ii) in the constant domain CL of the first antigen binding domain the amino acid at position 124 is substituted by a positively charged amino acid (numbering according to Kabat), and wherein in the constant domain CH1 of the first antigen binding domain the amino acid at position 147 or the amino acid at position 213 is substituted by a negatively charged amino acid (numbering according to Kabat EU index).

The (multispecific) antibody does not comprise both modifications mentioned under i) and ii). The constant domains CL and CH1 of the antigen binding domain having the VH/VL exchange are not replaced by each other (i.e. remain unexchanged).

In a more specific aspect,
i) in the constant domain CL of the second and, where present, the third antigen binding domain the amino acid at position 124 is substituted independently by lysine (K), arginine (R) or histidine (H) (numbering according to Kabat), and in the constant domain CH1 of the second and, where present, the third antigen binding domain the amino acid at position 147 or the amino acid at position 213 is substituted independently by glutamic acid (E), or aspartic acid (D) (numbering according to Kabat EU index); or
ii) in the constant domain CL of the first antigen binding domain the amino acid at position 124 is substituted independently by lysine (K), arginine (R) or histidine (H) (numbering according to Kabat), and in the constant domain CH1 of the first antigen binding domain the amino acid at position 147 or the amino acid at position 213 is substituted independently by glutamic acid (E), or aspartic acid (D) (numbering according to Kabat EU index).

In one such aspect, in the constant domain CL of the second and, where present, the third antigen binding domain the amino acid at position 124 is substituted independently by lysine (K), arginine (R) or histidine (H) (numbering according to Kabat), and in the constant domain CH1 of the second and, where present, the third antigen binding domain the amino acid at position 147 or the amino acid at position 213 is substituted independently by glutamic acid (E), or aspartic acid (D) (numbering according to Kabat EU index).

In a further aspect, in the constant domain CL of the second and, where present, the third antigen binding domain the amino acid at position 124 is substituted independently by lysine (K), arginine (R) or histidine (H) (numbering according to Kabat), and in the constant domain CH1 of the second and, where present, the third antigen binding domain the amino acid at position 147 is substituted independently by glutamic acid (E), or aspartic acid (D) (numbering according to Kabat EU index).

In a preferred aspect, in the constant domain CL of the second and, where present, the third antigen binding domain the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the constant domain CH1 of the second and, where present, the third antigen binding domain the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In a more preferred aspect, in the constant domain CL of the second and, where present, the third antigen binding domain the amino acid at position 124 is substituted by lysine (K) (numbering according to Kabat) and the amino acid at position 123 is substituted by lysine (K) (numbering according to Kabat), and in the constant domain CH1 of the second and, where present, the third antigen binding domain the amino acid at position 147 is substituted by glutamic acid (E) (numbering according to Kabat EU index) and the amino acid at position 213 is substituted by glutamic acid (E) (numbering according to Kabat EU index).

In an even more preferred aspect, in the constant domain CL of the second and, where present, the third antigen binding domain the amino acid at position 124 is substituted by lysine (K) (numbering according to Kabat) and the amino acid at position 123 is substituted by arginine (R) (numbering according to Kabat), and in the constant domain CH1 of the second and, where present, the third antigen binding domain the amino acid at position 147 is substituted by glutamic acid (E) (numbering according to Kabat EU index) and the amino acid at position 213 is substituted by glutamic acid (E) (numbering according to Kabat EU index).

In preferred aspects, if amino acid substitutions according to the above aspects are made in the constant domain CL and the constant domain CH1 of the second and, where present, the third antigen binding domain, the constant domain CL of the second and, where present, the third antigen binding domain is of kappa isotype.

Alternatively, the amino acid substitutions according to the above aspects may be made in the constant domain CL and the constant domain CH1 of the first antigen binding domain instead of in the constant domain CL and the constant domain CH1 of the second and, where present, the third antigen binding domain. In preferred such aspects, the constant domain CL of the first antigen binding domain is of kappa isotype.

Accordingly, in one aspect, in the constant domain CL of the first antigen binding domain the amino acid at position 124 is substituted independently by lysine (K), arginine (R) or histidine (H) (numbering according to Kabat), and in the constant domain CH1 of the first antigen binding domain the amino acid at position 147 or the amino acid at position 213 is substituted independently by glutamic acid (E), or aspartic acid (D) (numbering according to Kabat EU index).

In a further aspect, in the constant domain CL of the first antigen binding domain the amino acid at position 124 is substituted independently by lysine (K), arginine (R) or histidine (H) (numbering according to Kabat), and in the constant domain CH1 of the first antigen binding domain the amino acid at position 147 is substituted independently by glutamic acid (E), or aspartic acid (D) (numbering according to Kabat EU index).

In still another aspect, in the constant domain CL of the first antigen binding domain the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the constant domain CH1 of the first antigen binding domain the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In one aspect, in the constant domain CL of the first antigen binding domain the amino acid at position 124 is substituted by lysine (K) (numbering according to Kabat) and the amino acid at position 123 is substituted by lysine (K) (numbering according to Kabat), and in the constant domain CH1 of the first antigen binding domain the amino acid at position 147 is substituted by glutamic acid (E) (numbering according to Kabat EU index) and the amino acid at position 213 is substituted by glutamic acid (E) (numbering according to Kabat EU index).

In another aspect, in the constant domain CL of the first antigen binding domain the amino acid at position 124 is substituted by lysine (K) (numbering according to Kabat) and the amino acid at position 123 is substituted by arginine (R) (numbering according to Kabat), and in the constant domain CH1 of the first antigen binding domain the amino acid at position 147 is substituted by glutamic acid (E) (numbering according to Kabat EU index) and the amino acid at position 213 is substituted by glutamic acid (E) (numbering according to Kabat EU index).

In a preferred aspect, the first antigen binding domain of the (multispecific) antibody of the invention is a crossover Fab molecule wherein the variable domains VH and VL are exchanged/replaced by each other, and the second and, where present, the third antigen binding domain of the (multispecific) antibody of the invention is a conventional Fab molecule wherein in the constant domain CL the amino acid at position 124 is substituted by lysine (K) (numbering according to Kabat) and the amino acid at position 123 is substituted by arginine (R) (numbering according to Kabat), and in the constant domain CH1 the amino acid at position 147 is substituted by glutamic acid (E) (numbering according to Kabat EU index) and the amino acid at position 213 is substituted by glutamic acid (E) (numbering according to Kabat EU index).

In a specific aspect, the (multispecific) antibody of the invention comprises
(A) a first antigen binding domain that binds to CD3, wherein the first antigen binding domain is a Fab molecule wherein the variable domains VL and VH of the Fab light chain and the Fab heavy chain are replaced by each other, and comprises
(i) a heavy chain variable region (VH_{CD3}) comprising a heavy chain complementarity determining region (HCDR) 1 of SEQ ID NO: 1, a HCDR 2 of SEQ ID NO: 9, and a HCDR 3 of SEQ ID NO: 10, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 7 and/or (ii) a VH_{CD3} comprising the amino acid sequence of SEQ ID NO: 11, and a VL_{CD3} comprising the amino acid sequence of SEQ ID NO: 8;
(i) a heavy chain variable region (VH_{CD3}) comprising a heavy chain complementarity determining region (HCDR) 1 of SEQ ID NO: 1, a HCDR 2 of SEQ ID NO: 2, and a HCDR 3 of SEQ ID NO: 3, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 7 and/or (ii) a VH_{CD3} comprising the amino acid sequence of SEQ ID NO: 4, and a VL_{CD3} comprising the amino acid sequence of SEQ ID NO: 8; or
(i) a heavy chain variable region (VH_{CD3}) comprising a heavy chain complementarity determining region (HCDR) 1 of SEQ ID NO: 1, a HCDR 2 of SEQ ID NO: 2, and a HCDR 3 of SEQ ID NO: 10, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 13 and/or (ii) a VH_{CD3} comprising the amino acid sequence of SEQ ID NO: 12, and a VL_{CD3} comprising the amino acid sequence of SEQ ID NO: 14;
and
(B) a second and optionally a third antigen binding domain that bind to a second antigen, wherein the second and third antigen binding domain are each a (conventional) Fab molecule;
wherein in the constant domain CL of the second antigen binding domain and, where present, the third antigen binding domain the amino acid at position 124 is substituted independently by lysine (K), arginine (R) or histidine (H) (numbering according to Kabat) (in a preferred aspect by lysine (K)) and the amino acid at position 123 is substituted independently by lysine (K), arginine (R) or histidine (H) (numbering according to Kabat) (in a preferred aspect by lysine (K) or arginine (R)), and in the constant domain CH1 of the second antigen binding domain and, where present, the third antigen binding domain the amino acid at position 147 is substituted independently by glutamic acid (E) or aspartic acid (D) (numbering according to Kabat EU index) (in a preferred aspect by glutamic acid (E)) and the amino acid at position 213 is substituted independently by glutamic acid (E) or aspartic acid (D) (numbering according to Kabat EU index) (in a preferred aspect by glutamic acid (E)).

In a more specific aspect, the (multispecific) antibody of the invention comprises
(A) a first antigen binding domain that binds to CD3, wherein the first antigen binding domain is a Fab molecule wherein the variable domains VL and VH of the Fab light chain and the Fab heavy chain are replaced by each other, and comprises
(i) a heavy chain variable region (VH_{CD3}) comprising a heavy chain complementarity determining region (HCDR) 1 of SEQ ID NO: 1, a HCDR 2 of SEQ ID NO: 9, and a HCDR 3 of SEQ ID NO: 10, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 7 and/or (ii) a VH_{CD3} comprising the amino acid sequence of SEQ ID NO: 11, and a VL_{CD3} comprising the amino acid sequence of SEQ ID NO: 8;
(i) a heavy chain variable region (VH_{CD3}) comprising a heavy chain complementarity determining region (HCDR) 1 of SEQ ID NO: 1, a HCDR 2 of SEQ ID NO: 2, and a HCDR 3 of SEQ ID NO: 3, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 7 and/or (ii) a VH_{CD3} comprising the amino acid sequence of SEQ ID NO: 4, and a VL_{CD3} comprising the amino acid sequence of SEQ ID NO: 8; or
(i) a heavy chain variable region (VH_{CD3}) comprising a heavy chain complementarity determining region (HCDR) 1 of SEQ ID NO: 1, a HCDR 2 of SEQ ID NO: 2, and a HCDR 3 of SEQ ID NO: 10, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 13 and/or (ii) a VH_{CD3} comprising the amino acid sequence of SEQ ID NO: 12, and a VL_{CD3} comprising the amino acid sequence of SEQ ID NO: 14;
and
(B) a second and optionally a third antigen binding domain that bind to CD20, wherein the second and third antigen binding domain are each a (conventional) Fab molecule and comprise (i) a heavy chain variable region (VH_{CD20}) comprising a heavy chain complementarity determining region (HCDR) 1 of SEQ ID NO: 15, a HCDR 2 of SEQ ID NO: 16, and a HCDR 3 of SEQ ID NO: 17, and a light chain variable region (VL_{CD20}) comprising a light chain complementarity determining region (LCDR) 1 of SEQ ID NO: 19, a LCDR 2 of SEQ ID NO: 20 and a LCDR 3 of SEQ ID NO: 21 and/or (ii) a VH_{CD20} comprising the amino acid sequence of SEQ ID NO: 18, and a VL_{CD20} comprising the amino acid sequence of SEQ ID NO: 22;
wherein in the constant domain CL of the second antigen binding domain and, where present, the third antigen binding domain the amino acid at position 124 is substituted independently by lysine (K), arginine (R) or histidine (H) (numbering according to Kabat) (in a preferred aspect by lysine (K)) and the amino acid at position 123 is substituted independently by lysine (K), arginine (R) or histidine (H) (numbering according to Kabat) (in a preferred aspect by lysine (K) or arginine (R)), and in the constant domain CH1 of the second antigen binding domain and, where present, the third antigen binding domain the amino acid at position 147 is substituted independently by glutamic acid (E) or aspartic acid (D) (numbering according to Kabat EU index) (in a preferred aspect by glutamic acid (E)) and the amino acid at position 213 is substituted independently by glutamic acid (E) or aspartic acid (D) (numbering according to Kabat EU index) (in a preferred aspect by glutamic acid (E)).

### 10. Fc Domain

In preferred aspects, the (multispecific) antibody of the invention comprises an Fc domain composed of a first and a second subunit. The first and the second subunit of the Fc domain are capable of stable association.

The Fc domain of the (multispecific) antibody consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other.

In one aspect, the (multispecific) antibody comprises not more than one Fc domain.

In one aspect, the Fc domain of the (multispecific) antibody is an IgG Fc domain. In a preferred aspect, the Fc domain is an IgG₁ Fc domain. In another aspect, the Fc domain is an IgG₄ Fc domain. In a more specific aspect, the Fc domain is an IgG₄ Fc domain comprising an amino acid substitution at position S228 (Kabat EU index numbering), particularly the amino acid substitution S228P. This amino acid substitution reduces in vivo Fab arm exchange of IgG₄ antibodies (see Stubenrauch et al., Drug Metabolism and Disposition 38, 84-91 (2010)). In a further preferred aspect, the Fc domain is a human Fc domain. In an even more preferred aspect, the Fc domain is a human IgG₁ Fc domain. An exemplary sequence of a human IgG₁ Fc region is given in SEQ ID NO: 33. In one aspect, additionally the C-terminal lysine (Lys447) is present. In another aspect, the C-terminal glycine (Gly446) is absent. In such aspect, the C-terminal amino acid residue may be proline (Pro445) or proline amide (Pro445-NH₂).

The C-terminus of the Fc domain of an antibody provided herein may be a complete C-terminus ending with the amino acid residues PGK. The C-terminus of the Fc domain may also be a shortened C-terminus in which one or two of the C terminal amino acid residues have been removed. In one aspect, the C-terminus of the Fc domain is a shortened C-terminus ending with the amino acid residue P. In one aspect, the C-terminus of the Fc domain is a shortened C-terminus ending with the amino acid residues PG. In one aspect, an antibody comprising an Fc domain as specified herein, comprises the C-terminal glycine-lysine dipeptide (G446 and K447, Kabat EU numbering of amino acid positions). In one aspect, an antibody comprising an Fc domain as specified herein, comprises a C-terminal glycine residue (G446, Kabat EU numbering of amino acid positions).

The Fc domain may incorporate any of the modifications, singly or in combination, described hereinbelow in relation to Fc domain variants.

### a) Fc Domain Variants

### (1) Fc Domain Modifications Promotinq Heterodimerization

(Multispecific) antibodies according to the invention comprise different antigen binding domains, which may be fused to one or the other of the two subunits of the Fc domain, thus the two subunits of the Fc domain are typically comprised in two non-identical polypeptide chains. Recombinant co-expression of these polypeptides and subsequent dimerization leads to several possible combinations of the two polypeptides. To improve the yield and purity of (multispecific) antibodies in recombinant production, it will thus be advantageous to introduce in the Fc domain of the (multispecific) antibody a modification promoting the association of the desired polypeptides.

Accordingly, in a preferred aspect, the Fc domain of the (multispecific) antibody according to the invention comprises a modification, particularly an amino acid substitution of combination of amino acid substitutions, promoting the association of the first and the second subunit of the Fc domain. The site of most extensive protein-protein interaction between the two subunits of a human IgG Fc domain is in the CH3 domain of the Fc domain. Thus, in one aspect said modification is in the CH3 domain of the Fc domain.

There exist several approaches for modifications in the CH3 domain of the Fc domain in order to enforce heterodimerization, which are well described e.g. in WO 96/27011, WO 98/050431, EP 1870459, WO 2007/110205, WO 2007/147901, WO 2009/089004, WO 2010/129304, WO 2011/90754, WO 2011/143545, WO 2012058768, WO 2013157954, WO 2013096291. Typically, in all such approaches the CH3 domain of the first subunit of the Fc domain and the CH3 domain of the second subunit of the Fc domain are both engineered in a complementary manner so that each CH3 domain (or the heavy chain comprising it) can no longer homodimerize with itself but is forced to heterodimerize with the complementarily engineered other CH3 domain (so that the first and second CH3 domain heterodimerize and no homodimers between two first or two second CH3 domains are formed). These different approaches for improved heavy chain heterodimerization are contemplated as different alternatives in combination with the heavy-light chain modifications (e.g. VH and VL exchange/replacement and/or introduction of substitutions of charged amino acids with opposite charges in the CH1/CL interface) in the (multispecific) antibody which reduce heavy/light chain mispairing and Bence Jones-type side products.

In a specific aspect, said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain.

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in a preferred aspect, in the CH3 domain of the first subunit of the Fc domain of the (multispecific) antibody an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

Preferably said amino acid residue having a larger side chain volume is selected from the group consisting of arginine (R), phenylalanine (F), tyrosine (Y), and tryptophan (W).

Preferably said amino acid residue having a smaller side chain volume is selected from the group consisting of alanine (A), serine (S), threonine (T), and valine (V).

The protuberance and cavity can be made by altering the nucleic acid encoding the polypeptides, e.g. by site-specific mutagenesis, or by peptide synthesis.

In a specific aspect, in (the CH3 domain of) the first subunit of the Fc domain (the “knobs” subunit) the threonine residue at position 366 is replaced with a tryptophan residue (T366W), and in (the CH3 domain of) the second subunit of the Fc domain (the “hole” subunit) the tyrosine residue at position 407 is replaced with a valine residue (Y407V). In one aspect, in the second subunit of the Fc domain additionally the threonine residue at position 366 is replaced with a serine residue (T366S) and the leucine residue at position 368 is replaced with an alanine residue (L368A) (numberings according to Kabat EU index).

In yet a further aspect, in the first subunit of the Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second subunit of the Fc domain additionally the tyrosine residue at position 349 is replaced by a cysteine residue (Y349C) (numberings according to Kabat EU index).

Introduction of these two cysteine residues results in formation of a disulfide bridge between the two subunits of the Fc domain, further stabilizing the dimer (Carter, J Immunol Methods 248, 7-15 (2001)).

In a preferred aspect, the first subunit of the Fc domain comprises the amino acid substitutions S354C and T366W, and the second subunit of the Fc domain comprises the amino acid substitutions Y349C, T366S, L368A and Y407V (numbering according to Kabat EU index).

In a preferred aspect, the antigen binding domain that binds to CD3 is fused to the first subunit of the Fc domain (comprising the “knob” modification). Without wishing to be bound by theory, fusion of the antigen binding domain that binds CD3 to the knob-containing subunit of the Fc domain will (further) minimize the generation of antibodies comprising two antigen binding domains that bind to CD3 (steric clash of two knob-containing polypeptides).

Other techniques of CH3-modification for promoting the heterodimerization of Fc domain subunits are also contemplated according to the invention.

For example, in an alternative aspect, a modification promoting association of the first and the second subunit of the Fc domain comprises a modification mediating electrostatic steering effects, e.g. as described in PCT publication WO 2009/089004 (incorporated herein by reference in its entirety).

Generally, this method involves replacement of one or more amino acid residues at the interface of the two Fc domain subunits by charged amino acid residues so that homodimer formation becomes electrostatically unfavorable but heterodimerization electrostatically favorable. In one such aspect, a first CH3 domain comprises amino acid substitution of K392 or N392 with a negatively charged amino acid (e.g. glutamic acid (E), or aspartic acid (D), particularly K392D or N392D) and a second CH3 domain comprises amino acid substitution of D399, E356, D356, or E357 with a positively charged amino acid (e.g. lysine (K) or arginine (R), particularly D399K, E356K, D356K, or E357K, and more particularly D399K and E356K). In a further aspect, the first CH3 domain further comprises amino acid substitution of K409 or R409 with a negatively charged amino acid (e.g. glutamic acid (E), or aspartic acid (D), particularly K409D or R409D). In a further aspect the first CH3 domain further or alternatively comprises amino acid substitution of K439 and/or K370 with a negatively charged amino acid (e.g. glutamic acid (E), or aspartic acid (D)) (all numberings according to Kabat EU index).

(2) Fc Domain Modifications Reducing Fcγ Receptor Binding and/or Effector Function

The Fc domain confers to the (multispecific) antibody favorable pharmacokinetic properties, including a long serum half-life which contributes to good accumulation in the target tissue and a favorable tissue-blood distribution ratio. At the same time it may, however, lead to undesirable targeting of the (multispecific) antibody to cells expressing Fc receptors rather than to the preferred antigen-bearing cells. Moreover, the co-activation of Fc receptor signaling pathways may lead to cytokine release which may lead to side effects upon systemic administration. Activation of (Fc receptor-bearing) immune cells other than T cells may even reduce efficacy of the (multispecific) antibody due to the potential destruction of T cells e.g. by NK cells.

Accordingly, in a preferred aspect, the Fc domain of the (multispecific) antibody according to the invention exhibits reduced binding affinity to an Fc receptor and/or reduced effector function, as compared to a native IgG₁ Fc domain. In one such aspect, the Fc domain (or the (multispecific) antibody comprising said Fc domain) exhibits less than 50%, particularly less than 20%, more particularly less than 10% and most particularly less than 5% of the binding affinity to an Fc receptor, as compared to a native IgG₁ Fc domain (or a (multispecific) antibody comprising a native IgG₁ Fc domain), and/or less than 50%, particularly less than 20%, more particularly less than 10% and most particularly less than 5% of the effector function, as compared to a native IgG₁ Fc domain (or a (multispecific) antibody comprising a native IgG₁ Fc domain). In one aspect, the Fc domain (or the (multispecific) antibody comprising said Fc domain) does not substantially bind to an Fc receptor and/or induce effector function. In a preferred aspect, the Fc receptor is an Fcγ receptor. In one aspect, the Fc receptor is a human Fc receptor. In one aspect, the Fc receptor is an activating Fc receptor. In a specific aspect, the Fc receptor is an activating human Fcγ receptor, more specifically human FcγRIIIa, FcγRI or FcγRIIa, most specifically human FcγRIIIa.

In one aspect, the effector function is one or more selected from the group of CDC, ADCC, ADCP, and cytokine secretion. In a preferred aspect, the effector function is ADCC. In one aspect, the Fc domain exhibits substantially similar binding affinity to neonatal Fc receptor (FcRn), as compared to a native IgG₁ Fc domain. Substantially similar binding to FcRn is achieved when the Fc domain (or the (multispecific) antibody comprising said Fc domain) exhibits greater than about 70%, particularly greater than about 80%, more particularly greater than about 90% of the binding affinity of a native IgG₁ Fc domain (or the (multispecific) antibody comprising a native IgG₁ Fc domain) to FcRn.

In one aspect, the Fc domain of the (multispecific) antibody is engineered to have reduced binding affinity to an Fc receptor and/or reduced effector function, as compared to a non-engineered Fc domain. In a preferred aspect, the Fc domain of the (multispecific) antibody comprises a modification, particulary an amino acid substitution or combination of amino acid substitutions, that reduces the binding affinity to an Fc receptor and/or effector function of the antibody, as compared to a corresponding antibody comprising an Fc domain without such modification. In one such aspect, the Fc receptor is an Fcγ receptor. In one aspect, the Fc receptor is a human Fc receptor. In one aspect, the Fc receptor is an activating Fc receptor. In a specific such aspect, the Fc receptor is an activating human Fcγ receptor, more specifically human FcγRIIIa, FcγRI or FcγRIIa, most specifically human FcγRIIIa. Typically, the same one or more amino acid mutation is present in each of the two subunits of the Fc domain. In one aspect, the amino acid mutation reduces the binding affinity of the Fc domain to an Fc receptor. In one aspect, the amino acid mutation reduces the binding affinity of the Fc domain to an Fc receptor by at least 2-fold, at least 5-fold, or at least 10-fold. In aspects where there is more than one amino acid mutation that reduces the binding affinity of the Fc domain to the Fc receptor, the combination of these amino acid mutations may reduce the binding affinity of the Fc domain to an Fc receptor by at least 10-fold, at least 20-fold, or even at least 50-fold. In one aspect, the (multispecific) antibody comprising an engineered Fc domain exhibits less than 20%, particularly less than 10%, more particularly less than 5% of the binding affinity to an Fc receptor as compared to a (multispecific) antibody comprising a non-engineered Fc domain. In a preferred aspect, the Fc receptor is an Fcγ receptor. In one aspect, the Fc receptor is a human Fc receptor. In one aspect, the Fc receptor is an activating Fc receptor. In a specific aspect, the Fc receptor is an activating human Fcγ receptor, more specifically human FcγRIIIa, FcγRI or FcγRIIa, most specifically human FcγRIIIa. Preferably, binding to each of these receptors is reduced. In one aspect, binding affinity to a complement component, specifically binding affinity to C1q, is also reduced. In one aspect, binding affinity to neonatal Fc receptor (FcRn) is not reduced. Substantially similar binding to FcRn, i.e. preservation of the binding affinity of the Fc domain to said receptor, is achieved when the Fc domain (or the (multispecific) antibody comprising said Fc domain) exhibits greater than about 70% of the binding affinity of a non-engineered form of the Fc domain (or the (multispecific) antibody comprising said non-engineered form of the Fc domain) to FcRn. The Fc domain, or a (multispecific) antibody comprising said Fc domain, may exhibit greater than about 80% and even greater than about 90% of such affinity. In one aspect, the Fc domain of the (multispecific) antibody is engineered to have reduced effector function, as compared to a non-engineered Fc domain. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced crosslinking of target-bound antibodies, reduced dendritic cell maturation, or reduced T cell priming. In one aspect, the reduced effector function is one or more selected from the group of reduced CDC, reduced ADCC, reduced ADCP, and reduced cytokine secretion. In a preferred aspect, the reduced effector function is reduced ADCC. In one aspect the reduced ADCC is less than 20% of the ADCC induced by a non-engineered Fc domain (or a (multispecific) antibody comprising a non-engineered Fc domain).

In one aspect, the amino acid mutation that reduces the binding affinity of the Fc domain to an Fc receptor and/or effector function is an amino acid substitution. In one aspect, the Fc domain comprises an amino acid substitution at one or more position selected from the group of E233, L234, L235, N297, P331 and P329 (numberings according to Kabat EU index). In a more specific aspect, the Fc domain comprises an amino acid substitution at one or more position selected from the group of L234, L235 and P329 (numberings according to Kabat EU index). In some aspects, the Fc domain comprises the amino acid substitutions L234A and L235A (numberings according to Kabat EU index). In one such aspect, the Fc domain is an IgG₁ Fc domain, particularly a human IgG₁ Fc domain. In one aspect, the Fc domain comprises an amino acid substitution at position P329. In a more specific aspect, the amino acid substitution is P329A or P329G, particularly P329G (numberings according to Kabat EU index). In one aspect, the Fc domain comprises an amino acid substitution at position P329 and a further amino acid substitution at a position selected from E233, L234, L235, N297 and P331 (numberings according to Kabat EU index). In a more specific aspect, the further amino acid substitution is E233P, L234A, L235A, L235E, N297A, N297D or P331S. In a preferred aspect, the Fc domain comprises amino acid substitutions at positions P329, L234 and L235 (numberings according to Kabat EU index). In a more preferred aspect, the Fc domain comprises the amino acid mutations L234A, L235A and P329G (“P329G LALA”, “PGLALA” or “LALAPG”). Specifically, in a preferred aspect, each subunit of the Fc domain comprises the amino acid substitutions L234A, L235A and P329G (Kabat EU index numbering), i.e. in each of the first and the second subunit of the Fc domain the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index).

In one such aspect, the Fc domain is an IgG₁ Fc domain, particularly a human IgG₁ Fc domain.

The “P329G LALA” combination of amino acid substitutions almost completely abolishes Fcγ receptor (as well as complement) binding of a human IgG₁ Fc domain, as described in PCT publication no. WO 2012/130831 or in Schlothauer et al., Protein Eng Des Sel 29, 457-466 (2016), which are both incorporated herein by reference in its entirety. WO 2012/130831 and Schlothauer et al. also describes methods of preparing such mutant Fc domains and methods for determining its properties such as Fc receptor binding or effector functions.

IgG₄ antibodies exhibit reduced binding affinity to Fc receptors and reduced effector functions as compared to IgGᵢ antibodies. Hence, in some aspects, the Fc domain of the (multispecific) antibody of the invention is an IgG₄ Fc domain, particularly a human IgG₄ Fc domain. In one aspect, the IgG₄ Fc domain comprises an amino acid substitution at position S228, specifically the amino acid substitution S228P (numberings according to Kabat EU index). To further reduce its binding affinity to an Fc receptor and/or its effector function, in one aspect, the IgG₄ Fc domain comprises an amino acid substitution at position L235, specifically the amino acid substitution L235E (numberings according to Kabat EU index). In another aspect, the IgG₄ Fc domain comprises an amino acid substitution at position P329, specifically the amino acid substitution P329G (numberings according to Kabat EU index). In a preferred aspect, the IgG₄ Fc domain comprises amino acid substitutions at positions S228, L235 and P329, specifically amino acid substitutions S228P, L235E and P329G (numberings according to Kabat EU index). Such IgG₄ Fc domain mutants and their Fcγ receptor binding properties are described in PCT publication no. WO 2012/130831 or in Schlothauer et al., Protein Eng Des Sel 29, 457-466 (2016), both incorporated herein by reference in its entirety.

In a preferred aspect, the Fc domain exhibiting reduced binding affinity to an Fc receptor and/or reduced effector function, as compared to a native IgG₁ Fc domain, is a human IgG₁ Fc domain comprising the amino acid substitutions L234A, L235A and optionally P329G, or a human IgG₄ Fc domain comprising the amino acid substitutions S228P, L235E and optionally P329G (numberings according to Kabat EU index).

Other Fc domain modifications for reducing Fc receptor binding and/or effector function are also contemplated according to the invention.

For example, in one aspect, N-glycosylation of the Fc domain has been eliminated. In one such aspect, the Fc domain comprises an amino acid mutation at position N297, particularly an amino acid substitution replacing asparagine by alanine (N297A), aspartic acid (N297D), glutamine (N297Q) or glycine (N297G) (numberings according to Kabat EU index). In one aspect, the Fc domain further comprises an amino acid substitution at position D265A (numbering according to Kabat EU index). In one aspect, the substitutions are D265A and N297A (DANA), or D265A and N297G (DANG). In such aspect, the Fc domain is a human IgG₁ Fc domain.

In another aspect, the Fc domain, particularly a human IgG₁ Fc domain, comprises the amino acid substitutions E233P, L234V, L235A and the amino acid deletion G236del (see e.g. Armour et al., Eur.

J. Immunol. 29, 2613-2624 (1999)). In one aspect, the Fc domain further comprises a N297G or S267K substitution (numberings according to Kabat EU index).

In yet another aspect, the Fc domain, particularly a human IgG₁ Fc domain, comprises the amino acid substitutions L234F, L235E and D265A (FEA), or L234F, L235E and P331S (FES) (numberings according to Kabat EU index).

Mutant Fc domains can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fc receptors can be easily determined e.g. by ELISA, or by Surface Plasmon Resonance (SPR) using standard instrumentation such as a BIAcore instrument (Cytiva), and Fc receptors such as may be obtained by recombinant expression. Alternatively, binding affinity of Fc domains or antibodies comprising an Fc domain for Fc receptors may be evaluated using cell lines known to express particular Fc receptors, such as human NK cells expressing FcγIIia receptor.

Effector function of an Fc domain, or a (multispecific) antibody comprising an Fc domain, can be measured by methods known in the art. Examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays may be employed (see, for example, ACTI™ non-radioactive cytotoxicity assay for flow cytometry (CellTechnology, Inc. Mountain View, CA); and CytoTox 96′ non-radioactive cytotoxicity assay (Promega, Madison, WI)). Useful effector cells for such assays include peripheral blood mononuclear cells (PBMC) and Natural Killer (NK) cells. Alternatively, or additionally, ADCC activity of the molecule of interest may be assessed in vivo, e.g. in an animal model such as that disclosed in Clynes et al., Proc Natl Acad Sci USA 95, 652-656 (1998).

In some aspects, binding of the Fc domain to a complement component, specifically to C1q, is reduced. Accordingly, in some aspects wherein the Fc domain is engineered to have reduced effector function, said reduced effector function includes reduced CDC. C1q binding assays may be carried out to determine whether the Fc domain, or the (multispecific) antibody comprising the Fc domain, is able to bind C1q and hence has CDC activity. See e.g., C1q and C3c binding ELISA in WO 2006/029879 and WO 2005/100402. To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)).

(3) Fc Domain Modifications Increasing FcRn Binding and/or Serum Half-Life

It may be desirable to further prolong serum half-life of the (multispecific) antibody of the invention, by introducing modifications in the Fc domain which increase binding to FcRn and/or serum half-life of the antibody. The benefits of such prolonged half-life include increased convenience in therapeutic application in that it allows extending the dosing interval (i.e. the time between subsequent administrations) of the antibody.

Accordingly, in one aspect, the Fc domain of the (multispecific) antibody according to the invention comprises a modification, particularly an amino acid substitution or combination of amino acid substitutions, that increases the binding affinity to an FcRn receptor and/or the serum half-life of the antibody, as compared to a corresponding antibody comprising an Fc domain without such modification. In a particular aspect, the FcRn receptor is a human FcRn receptor. In one aspect, the Fc domain comprises an amino acid substitution at one or more position selected from the group of 252, 254, 256, 428 and 434 (numberings according to Kabat EU index).

In a more specific aspect, the Fc domain comprises an amino acid substitution at one or more position selected from the group of 252, 254 and 256 (numberings according to Kabat EU index). In a preferred aspect, the Fc domain (in each of its subunits) comprises the amino acid substitutions M252Y, S254T and T256E (“YTE”; numberings according to Kabat EU index). See, e.g., Dall'Acqua et al. J Biol Chem 281, 23514-23524 (2006) or WO 2002/60919 (both incorporated by reference herein in their entirety). In such aspect, the Fc domain is an IgG₁ Fc domain, particularly a human IgG₁ Fc domain.

In another aspect, the Fc domain comprises an amino acid substitution at one or more position selected from the group of 428 and 434 (numberings according to Kabat EU index). In a preferred aspect, the Fc domain (in each of its subunits) comprises the amino acid substitutions M428L and N434S (“LS”; numberings according to Kabat EU index). See e.g. Zalevsky et al. Nat Biotech 28, 157-159 (2010) or WO 2009/086320 (both incorporated by reference herein in their entirety). In one aspect, the Fc domain (in each of its subunits) comprises the amino acid substitutions M428L and N434A (“LA”; numberings according to Kabat EU index). In such aspects, the Fc domain is an IgG₁ Fc domain, particularly a human IgG₁ Fc domain.

Fc domain modifications that increase FcRn binding and/or serum half-life may be combined with modifications that reduce binding to rheumatoid factor. Such modifications include e.g. amino acid substitutions at positions 424, 436, 438 and/or 440 (Kabat EU numbering of residues). Accordingly, in one aspect, the Fc domain of the (multispecific) antibody according to the invention comprises (i) an amino acid mutation, particularly an amino acid substitution, that increases the binding affinity of the Fc domain to an FcRn receptor and/or the serum half-life of the antibody as described hereinabove, and (ii) an amino acid mutation, particularly an amino acid substitution, that reduces the binding affinity of the Fc domain to rheumatoid factor. In a particular aspect, the rheumatoid factor is human rheumatoid factor.

In one aspect, the Fc domain (in each of its subunits) comprises the amino acid substitutions M428L, N434A and Y436T, or M428L, N434S and Y436T (numberings according to Kabat EU index). In one aspect, the Fc domain (in each of its subunits) comprises the amino acid substitutions N434A, Q438R, S440E, and optionally Y436T or Y436V (numberings according to Kabat EU index). In one aspect, the Fc domain (in each of its subunits) comprises the amino acid substitutions M428L, N434A, Q438R, S440E, and optionally Y436T or Y436V (numberings according to Kabat EU index). In a specific aspect, the Fc domain (in each of its subunits) comprises the amino acid substitutions M428L, N434A, Q438R, S440E (“ACTS”; numberings according to Kabat EU index). See Maeda et al. MABS 9, 844-853 (2017) (incorporated herein by reference in its entirety). In such aspects, the Fc domain is an IgG₁ Fc domain, particularly a human IgG₁ Fc domain.

FcRn binding and in vivo clearance/half-life determinations can be performed using methods known in the art (see e.g., Petkova, S. B. et al., Int'l. Immunol. 18(12):1759-1769 (2006); WO 2013/120929).

In a preferred aspect, the Fc domain of the (multispecific) antibody of the invention is a human IgG₁ Fc domain comprising

in the first subunit the amino acid substitutions S354C and T366W, and in the second subunit the amino acid substitutions Y349C, T366S, L368A and Y407V;
in each of its subunits the amino acid substitutions L234A, L235A and P329G;
and optionally
in each of its subunits (a) the amino acid substitutions M252Y, S254T and T256E, (b) the amino acid substitutions M428L and N434, or (c) the amino acid substitutions M428L, N434A, Q438R and S440E (numberings according to Kabat EU index).

In a specific aspect, the (multispecific) antibody of the invention comprises
(A) a first antigen binding domain that binds to CD3, comprising
(b) (i) a heavy chain variable region (VH_{CD3}) comprising a heavy chain complementarity determining region (HCDR) 1 of SEQ ID NO: 1, a HCDR 2 of SEQ ID NO: 9, and a HCDR 3 of SEQ ID NO: 10, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 7 and/or (ii) a VH_{CD3} comprising the amino acid sequence of SEQ ID NO: 11, and a VL_{CD3} comprising the amino acid sequence of SEQ ID NO: 8;
(c) (i) a heavy chain variable region (VH_{CD3}) comprising a heavy chain complementarity determining region (HCDR) 1 of SEQ ID NO: 1, a HCDR 2 of SEQ ID NO: 2, and a HCDR 3 of SEQ ID NO: 3, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 7 and/or (ii) a VH_{CD3} comprising the amino acid sequence of SEQ ID NO: 4, and a VL_{CD3} comprising the amino acid sequence of SEQ ID NO: 8; or
(d) (i) a heavy chain variable region (VH_{CD3}) comprising a heavy chain complementarity determining region (HCDR) 1 of SEQ ID NO: 1, a HCDR 2 of SEQ ID NO: 2, and a HCDR 3 of SEQ ID NO: 10, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 13 and/or (ii) a VH_{CD3} comprising the amino acid sequence of SEQ ID NO: 12, and a VL_{CD3} comprising the amino acid sequence of SEQ ID NO: 14;
and
(B) a second and optionally a third antigen binding domain that bind to a second antigen;
(C) a human IgG₁ Fc domain composed of a first and a second subunit, comprising (i) in the first subunit the amino acid substitutions S354C and T366W, and in the second subunit the amino acid substitutions Y349C, T366S, L368A and Y407V; (ii) in each of its subunits the amino acid substitutions L234A, L235A and P329G; and optionally (iii) in each of its subunits (a) the amino acid substitutions M252Y, S254T and T256E, (b) the amino acid substitutions M428L and N434, or (c) the amino acid substitutions M428L, N434A, Q438R and S440E (numberings according to Kabat EU index).

In a more specific aspect, the (multispecific) antibody of the invention comprises
(A) a first antigen binding domain that binds to CD3, comprising
(i) a heavy chain variable region (VH_{CD3}) comprising a heavy chain complementarity determining region (HCDR) 1 of SEQ ID NO: 1, a HCDR 2 of SEQ ID NO: 9, and a HCDR 3 of SEQ ID NO: 10, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 7 and/or (ii) a VH_{CD3} comprising the amino acid sequence of SEQ ID NO: 11, and a VL_{CD3} comprising the amino acid sequence of SEQ ID NO: 8;
(i) a heavy chain variable region (VH_{CD3}) comprising a heavy chain complementarity determining region (HCDR) 1 of SEQ ID NO: 1, a HCDR 2 of SEQ ID NO: 2, and a HCDR 3 of SEQ ID NO: 3, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 7 and/or (ii) a VH_{CD3} comprising the amino acid sequence of SEQ ID NO: 4, and a VL_{CD3} comprising the amino acid sequence of SEQ ID NO: 8; or
(i) a heavy chain variable region (VH_{CD3}) comprising a heavy chain complementarity determining region (HCDR) 1 of SEQ ID NO: 1, a HCDR 2 of SEQ ID NO: 2, and a HCDR 3 of SEQ ID NO: 10, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 13 and/or (ii) a VH_{CD3} comprising the amino acid sequence of SEQ ID NO: 12, and a VL_{CD3} comprising the amino acid sequence of SEQ ID NO: 14;
and
(B) a second and optionally a third antigen binding domain that bind to CD20, comprising (i) a heavy chain variable region (VH_{CD20}) comprising a heavy chain complementarity determining region (HCDR) 1 of SEQ ID NO: 15, a HCDR 2 of SEQ ID NO: 16, and a HCDR 3 of SEQ ID NO: 17, and a light chain variable region (VL_{CD20}) comprising a light chain complementarity determining region (LCDR) 1 of SEQ ID NO: 19, a LCDR 2 of SEQ ID NO: 20 and a LCDR 3 of SEQ ID NO: 21 and/or (ii) a VH_{CD20} comprising the amino acid sequence of SEQ ID NO: 18, and a VL_{CD20} comprising the amino acid sequence of SEQ ID NO: 22;
(C) a human IgG₁ Fc domain composed of a first and a second subunit, comprising (i) in the first subunit the amino acid substitutions S354C and T366W, and in the second subunit the amino acid substitutions Y349C, T366S, L368A and Y407V; (ii) in each of its subunits the amino acid substitutions L234A, L235A and P329G; and optionally (iii) in each of its subunits (a) the amino acid substitutions M252Y, S254T and T256E, (b) the amino acid substitutions M428L and N434, or (c) the amino acid substitutions M428L, N434A, Q438R and S440E (numberings according to Kabat EU index).

### 11. Multispecific Antibody Formats

The (multispecific) antibody according to the invention can have different configurations, i.e. the first, the second and, where present, the third antigen binding domain may be fused to each other and/or to the Fc domain in different ways. The components may be fused to each other directly or, preferably, via one or more suitable peptide linkers. Where fusion of a Fab molecule is to the N-terminus of a subunit of the Fc domain, it is typically via an immunoglobulin hinge region.

In a preferred aspect, the antigen binding domains comprised in the (multispecific) antibody are Fab molecules. In such aspect, the first, second, third etc. antigen binding domain may be referred to herein as first, second, third etc. Fab molecule, respectively.

In one aspect, the first and the second antigen binding domain of the (multispecific) antibody are fused to each other, optionally via a peptide linker. In a preferred aspect, the first and the second antigen binding domain are each a Fab molecule. In one such aspect, the first antigen binding domain is fused at the C-terminus of the Fab heavy chain to the N-terminus of the Fab heavy chain of the second antigen binding domain. In another such aspect, the second antigen binding domain is fused at the C-terminus of the Fab heavy chain to the N-terminus of the Fab heavy chain of the first antigen binding domain.

A (multispecific) antibody with a single antigen binding domain (such as a Fab molecule) capable of specific binding to a second antigen, e.g. a target cell antigen such as CD20, is useful, particularly in cases where internalization of the second antigen is to be expected following binding of a high affinity antigen binding domain. In such cases, the presence of more than one antigen binding domain specific for the second antigen may enhance internalization of the second antigen, thereby reducing its availability.

In other cases, however, it will be advantageous to have a (multispecific) antibody comprising two or more antigen binding domains (such as Fab molecules) specific for a second antigen, e.g. a target cell antigen, for example to optimize targeting to the target site or to allow crosslinking of target cell antigens.

Accordingly, in a preferred aspect, the (multispecific) antibody according to the present invention comprises a third antigen binding domain, which binds to the second antigen, e.g. a target cell antigen such as CD20. In one aspect, the third antigen binding domain is a Fab molecule.

In one aspect, the third antigen binding domain is identical to the second antigen binding domain.

In one aspect, the second and the third antigen binding domain are each a Fab molecule and the third antigen binding domain is identical to the second antigen binding domain. Thus, in such aspect, the second and the third antigen binding domain comprise the same heavy and light chain amino acid sequences and have the same arrangement of domains (i.e. conventional or crossover). Furthermore, in such aspect, the third antigen binding domain comprises the same amino acid substitutions, if any, as the second antigen binding domain. For example, the amino acid substitutions described herein as “charge modifications” will be made in the constant domain CL and the constant domain CH1 of each of the second antigen binding domain and the third antigen binding domain. Alternatively, said amino acid substitutions may be made in the constant domain CL and the constant domain CH1 of the first antigen binding domain (which in preferred aspects is also a Fab molecule), but not in the constant domain CL and the constant domain CH1 of the second antigen binding domain and the third antigen binding domain.

Like the second antigen binding domain, the third antigen binding domain preferably is a conventional Fab molecule. Aspects wherein the second and the third antigen binding domains are crossover Fab molecules (and the first antigen binding domain is a conventional Fab molecule) are, however, also contemplated. Thus, in a preferred aspect, the second and the third antigen binding domains are each a conventional Fab molecule, and the first antigen binding domain is a crossover Fab molecule as described herein, i.e. a Fab molecule wherein the variable domains VH and VL or the constant domains CL and CH1 of the Fab heavy and light chains are exchanged/replaced by each other.

In another aspect, the second and the third antigen binding domains are each a crossover Fab molecule and the first antigen binding domain is a conventional Fab molecule.

If a third antigen binding domain is present, in a preferred aspect the first antigen domain binds to CD3, and the second and third antigen binding domain bind to the second antigen, particularly a target cell antigen, such as CD20.

In a preferred aspect, the (multispecific) antibody of the invention comprises an Fc domain composed of a first and a second subunit. The first and the second subunit of the Fc domain are capable of stable association.

Thus, in a preferred aspect, the (multispecific) antibody of the invention comprises
(a) a first antigen binding domain that binds to CD3,
(b) a second and optionally a third antigen binding domain that bind to a second antigen, and
(c) an Fc domain composed of a first and a second subunit (all of (a)-(c) as described hereinabove).

In one aspect, the first and the second antigen binding domain are each a Fab molecule and the first antigen binding domain is fused at the C-terminus of the Fab heavy chain to the N-terminus of the first or the second subunit of the Fc domain. In such aspect, the second antigen binding domain may be fused at the C-terminus of the Fab heavy chain to the N-terminus of the Fab heavy chain of the first antigen binding domain or to the N-terminus of the other one of the subunits of the Fc domain. In a preferred such aspect, the second antigen binding domain is a conventional Fab molecule, and the first antigen binding domain is a crossover Fab molecule as described herein, i.e. a Fab molecule wherein the variable domains VH and VL or the constant domains CL and CH1 of the Fab heavy and light chains are exchanged/replaced by each other. In another such aspect, the second antigen binding domain is a crossover Fab molecule and the first antigen binding domain is a conventional Fab molecule.

In one aspect, the first and the second antigen binding domain are each a Fab molecule, the first antigen binding domain is fused at the C-terminus of the Fab heavy chain to the N-terminus of the first or the second subunit of the Fc domain, and the second antigen binding domain is fused at the C-terminus of the Fab heavy chain to the N-terminus of the Fab heavy chain of the first antigen binding domain. In a specific aspect, the (multispecific) antibody essentially consists of the first and the second Fab molecule, the Fc domain composed of a first and a second subunit, and optionally one or more peptide linkers, wherein the second Fab molecule is fused at the C-terminus of the Fab heavy chain to the N-terminus of the Fab heavy chain of the first Fab molecule, and the first Fab molecule is fused at the C-terminus of the Fab heavy chain to the N-terminus of the first or the second subunit of the Fc domain.

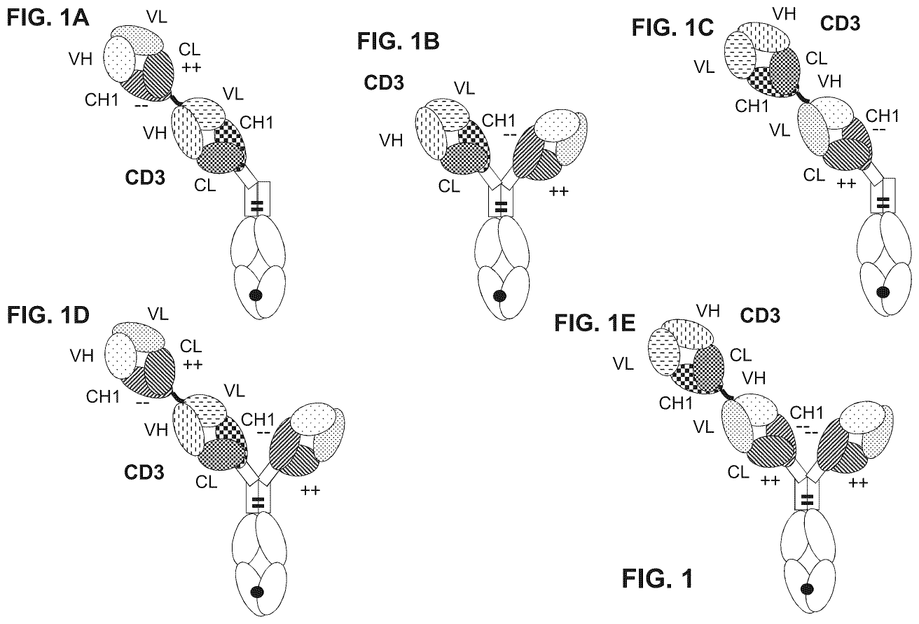

Such a configuration is schematically depicted in FIG. 1A (with the first antigen binding domain in these examples being a VH/VL crossover Fab molecule and the second antigen binding domain being a conventional Fab molecule).

In another aspect, the first and the second antigen binding domain are each a Fab molecule and the first and the second antigen binding domain are each fused at the C-terminus of the Fab heavy chain to the N-terminus of one of the subunits of the Fc domain. In a specific aspect, the (multispecific) antibody essentially consists of the first and the second Fab molecule, the Fc domain composed of a first and a second subunit, and optionally one or more peptide linkers, wherein the first and the second Fab molecule are each fused at the C-terminus of the Fab heavy chain to the N-terminus of one of the subunits of the Fc domain.

Such a configuration is schematically depicted in FIG. 1B (in this example with the first antigen binding domain being a VH/VL crossover Fab molecule and the second antigen binding domain being a conventional Fab molecule). The first and the second Fab molecule may be fused to the Fc domain directly or through a peptide linker. In a preferred aspect, the first and the second Fab molecule are each fused to the Fc domain through an immunoglobulin hinge region. In a specific aspect, the immunoglobulin hinge region is a human IgG₁ hinge region, particularly where the Fc domain is an IgG₁ Fc domain.

In one aspect, the first and the second antigen binding domain are each a Fab molecule and the second antigen binding domain is fused at the C-terminus of the Fab heavy chain to the N-terminus of the first or the second subunit of the Fc domain. In such aspects, the first antigen binding domain may be fused at the C-terminus of the Fab heavy chain to the N-terminus of the Fab heavy chain of the second antigen binding domain or (as described above) to the N-terminus of the other one of the subunits of the Fc domain. In a preferred such aspect, said second antigen binding domain is a conventional Fab molecule, and the first antigen binding domain is a crossover Fab molecule as described herein, i.e. a Fab molecule wherein the variable domains VH and VL or the constant domains CL and CH1 of the Fab heavy and light chains are exchanged/replaced by each other. In another such aspect, said second antigen binding domain is a crossover Fab molecule and the first antigen binding domain is a conventional Fab molecule.

In one aspect, the first and the second antigen binding domain are each a Fab molecule, the second antigen binding domain is fused at the C-terminus of the Fab heavy chain to the N-terminus of the first or the second subunit of the Fc domain, and the first antigen binding domain is fused at the C-terminus of the Fab heavy chain to the N-terminus of the Fab heavy chain of the second antigen binding domain. In a specific aspect, the (multispecific) antibody essentially consists of the first and the second Fab molecule, the Fc domain composed of a first and a second subunit, and optionally one or more peptide linkers, wherein the first Fab molecule is fused at the C-terminus of the Fab heavy chain to the N-terminus of the Fab heavy chain of the second Fab molecule, and the second Fab molecule is fused at the C-terminus of the Fab heavy chain to the N-terminus of the first or the second subunit of the Fc domain.

Such a configuration is schematically depicted in FIG. 1C (in this example with the first antigen binding domain being a VH/VL crossover Fab molecule and the second antigen binding domain being a conventional Fab molecule).

In one aspect, a third antigen binding domain, particularly a third Fab molecule, is fused at the C-terminus of the Fab heavy chain to the N-terminus of the first or second subunit of the Fc domain. In a preferred such aspect, the second and third antigen binding domains are each a conventional Fab molecule, and the first antigen binding domain is a crossover Fab molecule as described herein, i.e. a Fab molecule wherein the variable domains VH and VL or the constant domains CL and CH1 of the Fab heavy and light chains are exchanged/replaced by each other. In another such aspect, said second and third antigen binding domains are each a crossover Fab molecule and the first antigen binding domain is a conventional Fab molecule.

In a preferred such aspect, the first, second and third antigen binding domain are each a Fab molecule, the first and the third antigen binding domain are each fused at the C-terminus of the Fab heavy chain to the N-terminus of one of the subunits of the Fc domain, and the second antigen binding domain is fused at the C-terminus of the Fab heavy chain to the N-terminus of the Fab heavy chain of the first antigen binding domain. In a specific aspect, the (multispecific) antibody essentially consists of the first, the second and the third Fab molecule, the Fc domain composed of a first and a second subunit, and optionally one or more peptide linkers, wherein the second Fab molecule is fused at the C-terminus of the Fab heavy chain to the N-terminus of the Fab heavy chain of the first Fab molecule, and the first Fab molecule is fused at the C-terminus of the Fab heavy chain to the N-terminus of the first subunit of the Fc domain, and wherein the third Fab molecule is fused at the C-terminus of the Fab heavy chain to the N-terminus of the second subunit of the Fc domain.

Such a configuration is schematically depicted in FIG. 1D (in this example with the first antigen binding domain being a VH/VL crossover Fab molecule, and the second and the third antigen binding domain being a conventional Fab molecule). The first and the third Fab molecule may be fused to the Fc domain directly or through a peptide linker. In a preferred aspect, the first and the third Fab molecule are each fused to the Fc domain through an immunoglobulin hinge region. In a specific aspect, the immunoglobulin hinge region is a human IgG₁ hinge region, particularly where the Fc domain is an IgG₁ Fc domain.

In another such aspect, the first, second and third antigen binding domain are each a Fab molecule, the second and the third antigen binding domain are each fused at the C-terminus of the Fab heavy chain to the N-terminus of one of the subunits of the Fc domain, and the first antigen binding domain is fused at the C-terminus of the Fab heavy chain to the N-terminus of the Fab heavy chain of the second antigen binding domain. In a specific aspect, the (multispecific) antibody essentially consists of the first, the second and the third Fab molecule, the Fc domain composed of a first and a second subunit, and optionally one or more peptide linkers, wherein the first Fab molecule is fused at the C-terminus of the Fab heavy chain to the N-terminus of the Fab heavy chain of the second Fab molecule, and the second Fab molecule is fused at the C-terminus of the Fab heavy chain to the N-terminus of the first subunit of the Fc domain, and wherein the third Fab molecule is fused at the C-terminus of the Fab heavy chain to the N-terminus of the second subunit of the Fc domain.

Such a configuration is schematically depicted in FIG. 1E (in this example with the first antigen binding domain being a VH/VL crossover Fab molecule, and the second and the third antigen binding domain being a conventional Fab molecule) The second and the third antigen binding domain may be fused to the Fc domain directly or through a peptide linker. In a preferred aspect, the second and the third antigen binding domain are each fused to the Fc domain through an immunoglobulin hinge region.

In a specific aspect, the immunoglobulin hinge region is a human IgG₁ hinge region, particularly where the Fc domain is an IgG₁ Fc domain.

In configurations of the (multispecific) antibody wherein a Fab molecule is fused at the C-terminus of the Fab heavy chain to the N-terminus of each of the subunits of the Fc domain through an immunoglobulin hinge region, the two Fab molecules, the hinge regions and the Fc domain essentially form an immunoglobulin molecule. In one aspect, the immunoglobulin molecule is an IgG class immunoglobulin. In a particular aspect, the immunoglobulin is an IgG₁ subclass immunoglobulin. In another aspect, the immunoglobulin is an IgG₄ subclass immunoglobulin. In a further preferred aspect, the immunoglobulin is a human immunoglobulin. In another aspect, the immunoglobulin is a humanized immunoglobulin. In one aspect, the immunoglobulin comprises a human constant region, particularly a human Fc region. In one aspect, the immunoglobulin molecule is an IgG class, particularly an IgG₁ subclass, immunoglobulin molecule comprising a human CH1, CH2, CH3 and/or CL domain. Exemplary sequences of human constant domains are given in SEQ ID NOs 34 and 35 (human kappa and lambda CL domains, respectively) and SEQ ID NO: 36 (human IgG₁ heavy chain constant domains CH1-CH2-CH3). In one aspect, the immunoglobulin molecule comprises a light chain constant region comprising an amino acid sequence that is at least about 95%, 96%, 97%, 98%, 99% or 100% identical to the amino acid sequence of SEQ ID NO: 34 or SEQ ID NO: 35. In one aspect, the immunoglobulin molecule comprises a heavy chain constant region comprising an amino acid sequence that is at least about 95%, 96%, 97%, 98%, 99% or 100% identical to the amino acid sequence of SEQ ID NO: 36. Particularly, the heavy chain constant region may comprise amino acid mutations in the Fc domain as described herein.

The first and the second antigen binding domain may be fused to the Fc domain or to each other directly or through a peptide linker. Various linkers may be used, particularly peptide linkers comprising one or more amino acids, typically about 2-20 amino acids, that are described herein or are known in the art. Suitable, non-immunogenic peptide linkers include, for example, (G₄S)ₙ, (SG₄)ₙ, G₄(SG₄)ₙ or (G₄S)ₙG₅ peptide linkers, wherein n is generally an integer from 1 to 10, typically from 1 to 4. In one aspect, said peptide linker has a length of at least 5 amino acids, in one aspect a length of 5 to 10 amino acids, and in a further aspect of 10 to 50 amino acids. In one aspect, said peptide linker is (G₄S)₂. In another aspect, said peptide linker is G₄SG₅. In still another aspect, said peptide linker is G₄SG₄. A particularly suitable peptide linker for fusing the Fab heavy chains of the first and the second Fab molecule to each other is G₄SG₄, i.e. a linker having the amino acid sequence of SEQ ID NO: 37. Additionally or alternatively, linkers may comprise (a portion of) an immunoglobulin hinge region. Particularly where a Fab molecule is fused to the N-terminus of an Fc domain subunit, it may be fused via an immunoglobulin hinge region or a portion thereof, with or without an additional peptide linker.

In a specific aspect, the (multispecific) antibody of the invention comprises
(A) a first antigen binding domain that binds to CD3, comprising
(i) a heavy chain variable region (VH_{CD3}) comprising a heavy chain complementarity determining region (HCDR) 1 of SEQ ID NO: 1, a HCDR 2 of SEQ ID NO: 9, and a HCDR 3 of SEQ ID NO: 10, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 7 and/or (ii) a VH_{CD3} comprising the amino acid sequence of SEQ ID NO: 11, and a VL_{CD3} comprising the amino acid sequence of SEQ ID NO: 8;
(i) a heavy chain variable region (VH_{CD3}) comprising a heavy chain complementarity determining region (HCDR) 1 of SEQ ID NO: 1, a HCDR 2 of SEQ ID NO: 2, and a HCDR 3 of SEQ ID NO: 3, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 7 and/or (ii) a VH_{CD3} comprising the amino acid sequence of SEQ ID NO: 4, and a VL_{CD3} comprising the amino acid sequence of SEQ ID NO: 8; or
(i) a heavy chain variable region (VH_{CD3}) comprising a heavy chain complementarity determining region (HCDR) 1 of SEQ ID NO: 1, a HCDR 2 of SEQ ID NO: 2, and a HCDR 3 of SEQ ID NO: 10, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 13 and/or (ii) a VH_{CD3} comprising the amino acid sequence of SEQ ID NO: 12, and a VL_{CD3} comprising the amino acid sequence of SEQ ID NO: 14;
(A) a second antigen binding domain that binds to a second antigen; and
(B) an Fc domain composed of a first and a second subunit;
wherein the first and the second antigen binding domain are each a Fab molecule, and (i) the first antigen binding domain is fused at the C-terminus of the Fab heavy chain to the N-terminus of the first or the second subunit of the Fc domain, and the second antigen binding domain is fused at the C-terminus of the Fab heavy chain to the N-terminus of the Fab heavy chain of the first antigen binding domain, or (ii) the second antigen binding domain is fused at the C-terminus of the Fab heavy chain to the N-terminus of the first or the second subunit of the Fc domain, and the first antigen binding domain is fused at the C-terminus of the Fab heavy chain to the N-terminus of the Fab heavy chain of the second antigen binding domain.

In a more specific aspect, the (multispecific) antibody of the invention comprises
(A) a first antigen binding domain that binds to CD3, comprising
(i) a heavy chain variable region (VH_{CD3}) comprising a heavy chain complementarity determining region (HCDR) 1 of SEQ ID NO: 1, a HCDR 2 of SEQ ID NO: 9, and a HCDR 3 of SEQ ID NO: 10, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 7 and/or (ii) a VH_{CD3} comprising the amino acid sequence of SEQ ID NO: 11, and a VL_{CD3} comprising the amino acid sequence of SEQ ID NO: 8;
(i) a heavy chain variable region (VH_{CD3}) comprising a heavy chain complementarity determining region (HCDR) 1 of SEQ ID NO: 1, a HCDR 2 of SEQ ID NO: 2, and a HCDR 3 of SEQ ID NO: 3, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 7 and/or (ii) a VH_{CD3} comprising the amino acid sequence of SEQ ID NO: 4, and a VL_{CD3} comprising the amino acid sequence of SEQ ID NO: 8; or
(i) a heavy chain variable region (VH_{CD3}) comprising a heavy chain complementarity determining region (HCDR) 1 of SEQ ID NO: 1, a HCDR 2 of SEQ ID NO: 2, and a HCDR 3 of SEQ ID NO: 10, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 13 and/or (ii) a VH_{CD3} comprising the amino acid sequence of SEQ ID NO: 12, and a VL_{CD3} comprising the amino acid sequence of SEQ ID NO: 14;
(A) a second antigen binding domain that binds to CD20, comprising (i) a heavy chain variable region (VH_{CD20}) comprising a heavy chain complementarity determining region (HCDR) 1 of SEQ ID NO: 15, a HCDR 2 of SEQ ID NO: 16, and a HCDR 3 of SEQ ID NO: 17, and a light chain variable region (VL_{CD20}) comprising a light chain complementarity determining region (LCDR) 1 of SEQ ID NO: 19, a LCDR 2 of SEQ ID NO: 20 and a LCDR 3 of SEQ ID NO: 21 and/or (ii) a VH_{CD20} comprising the amino acid sequence of SEQ ID NO: 18, and a VL_{CD20} comprising the amino acid sequence of SEQ ID NO: 22; and
(B) an Fc domain composed of a first and a second subunit;
wherein the first and the second antigen binding domain are each a Fab molecule, and (i) the first antigen binding domain is fused at the C-terminus of the Fab heavy chain to the N-terminus of the first or the second subunit of the Fc domain, and the second antigen binding domain is fused at the C-terminus of the Fab heavy chain to the N-terminus of the Fab heavy chain of the first antigen binding domain, or (ii) the second antigen binding domain is fused at the C-terminus of the Fab heavy chain to the N-terminus of the first or the second subunit of the Fc domain, and the first antigen binding domain is fused at the C-terminus of the Fab heavy chain to the N-terminus of the Fab heavy chain of the second antigen binding domain.

In a further specific aspect, the (multispecific) antibody of the invention comprises
(A) a first antigen binding domain that binds to CD3, comprising
(i) a heavy chain variable region (VH_{CD3}) comprising a heavy chain complementarity determining region (HCDR) 1 of SEQ ID NO: 1, a HCDR 2 of SEQ ID NO: 9, and a HCDR 3 of SEQ ID NO: 10, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 7 and/or (ii) a VH_{CD3} comprising the amino acid sequence of SEQ ID NO: 11, and a VL_{CD3} comprising the amino acid sequence of SEQ ID NO: 8;
(i) a heavy chain variable region (VH_{CD3}) comprising a heavy chain complementarity determining region (HCDR) 1 of SEQ ID NO: 1, a HCDR 2 of SEQ ID NO: 2, and a HCDR 3 of SEQ ID NO: 3, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 7 and/or (ii) a VH_{CD3} comprising the amino acid sequence of SEQ ID NO: 4, and a VL_{CD3} comprising the amino acid sequence of SEQ ID NO: 8; or
(i) a heavy chain variable region (VH_{CD3}) comprising a heavy chain complementarity determining region (HCDR) 1 of SEQ ID NO: 1, a HCDR 2 of SEQ ID NO: 2, and a HCDR 3 of SEQ ID NO: 10, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 13 and/or (ii) a VH_{CD3} comprising the amino acid sequence of SEQ ID NO: 12, and a VL_{CD3} comprising the amino acid sequence of SEQ ID NO: 14;
(A) a second antigen binding domain that binds to a second antigen; and
(B) an Fc domain composed of a first and a second subunit;
wherein the first and the second antigen binding domain are each a Fab molecule, and the first and the second antigen binding domain are each fused at the C-terminus of the Fab heavy chain to the N-terminus of the first or the second subunit of the Fc domain.

In a more specific aspect, the (multispecific) antibody of the invention comprises
(A) a first antigen binding domain that binds to CD3, comprising
(i) a heavy chain variable region (VH_{CD3}) comprising a heavy chain complementarity determining region (HCDR) 1 of SEQ ID NO: 1, a HCDR 2 of SEQ ID NO: 9, and a HCDR 3 of SEQ ID NO: 10, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 7 and/or (ii) a VH_{CD3} comprising the amino acid sequence of SEQ ID NO: 11, and a VL_{CD3} comprising the amino acid sequence of SEQ ID NO: 8;
(i) a heavy chain variable region (VH_{CD3}) comprising a heavy chain complementarity determining region (HCDR) 1 of SEQ ID NO: 1, a HCDR 2 of SEQ ID NO: 2, and a HCDR 3 of SEQ ID NO: 3, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 7 and/or (ii) a VH_{CD3} comprising the amino acid sequence of SEQ ID NO: 4, and a VL_{CD3} comprising the amino acid sequence of SEQ ID NO: 8; or
(i) a heavy chain variable region (VH_{CD3}) comprising a heavy chain complementarity determining region (HCDR) 1 of SEQ ID NO: 1, a HCDR 2 of SEQ ID NO: 2, and a HCDR 3 of SEQ ID NO: 10, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 13 and/or (ii) a VH_{CD3} comprising the amino acid sequence of SEQ ID NO: 12, and a VL_{CD3} comprising the amino acid sequence of SEQ ID NO: 14;
(A) a second antigen binding domain that binds to CD20, comprising (i) a heavy chain variable region (VH_{CD20}) comprising a heavy chain complementarity determining region (HCDR) 1 of SEQ ID NO: 15, a HCDR 2 of SEQ ID NO: 16, and a HCDR 3 of SEQ ID NO: 17, and a light chain variable region (VL_{CD20}) comprising a light chain complementarity determining region (LCDR) 1 of SEQ ID NO: 19, a LCDR 2 of SEQ ID NO: 20 and a LCDR 3 of SEQ ID NO: 21 and/or (ii) a VH_{CD20} comprising the amino acid sequence of SEQ ID NO: 18, and a VL_{CD20} comprising the amino acid sequence of SEQ ID NO: 22; and
(B) an Fc domain composed of a first and a second subunit;
wherein the first and the second antigen binding domain are each a Fab molecule, and the first and the second antigen binding domain are each fused at the C-terminus of the Fab heavy chain to the N-terminus of the first or the second subunit of the Fc domain.

In yet another specific aspect, the (multispecific) antibody of the invention comprises (A) a first antigen binding domain that binds to CD3, comprising
(i) a heavy chain variable region (VH_{CD3}) comprising a heavy chain complementarity determining region (HCDR) 1 of SEQ ID NO: 1, a HCDR 2 of SEQ ID NO: 9, and a HCDR 3 of SEQ ID NO: 10, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 7 and/or (ii) a VH_{CD3} comprising the amino acid sequence of SEQ ID NO: 11, and a VL_{CD3} comprising the amino acid sequence of SEQ ID NO: 8;
(i) a heavy chain variable region (VH_{CD3}) comprising a heavy chain complementarity determining region (HCDR) 1 of SEQ ID NO: 1, a HCDR 2 of SEQ ID NO: 2, and a HCDR 3 of SEQ ID NO: 3, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 7 and/or (ii) a VH_{CD3} comprising the amino acid sequence of SEQ ID NO: 4, and a VL_{CD3} comprising the amino acid sequence of SEQ ID NO: 8; or
(i) a heavy chain variable region (VH_{CD3}) comprising a heavy chain complementarity determining region (HCDR) 1 of SEQ ID NO: 1, a HCDR 2 of SEQ ID NO: 2, and a HCDR 3 of SEQ ID NO: 10, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 13 and/or (ii) a VH_{CD3} comprising the amino acid sequence of SEQ ID NO: 12, and a VL_{CD3} comprising the amino acid sequence of SEQ ID NO: 14;
(A) a second and a third antigen binding domain that bind to a second antigen; and
(B) an Fc domain composed of a first and a second subunit;
wherein the first, the second and the third antigen binding domain are each a Fab molecule, and (i) the first and the third antigen binding domain are each fused at the C-terminus of the Fab heavy chain to the N-terminus of the first or the second subunit of the Fc domain, and the second antigen binding domain is fused at the C-terminus of the Fab heavy chain to the N-terminus of the Fab heavy chain of the first antigen binding domain, or (ii) the second and the third antigen binding domain are each fused at the C-terminus of the Fab heavy chain to the N-terminus of the first or the second subunit of the Fc domain, and the first antigen binding domain is fused at the C-terminus of the Fab heavy chain to the N-terminus of the Fab heavy chain of the second antigen binding domain.

In a more specific aspect, the (multispecific) antibody of the invention comprises
(A) a first antigen binding domain that binds to CD3, comprising
(i) a heavy chain variable region (VH_{CD3}) comprising a heavy chain complementarity determining region (HCDR) 1 of SEQ ID NO: 1, a HCDR 2 of SEQ ID NO: 9, and a HCDR 3 of SEQ ID NO: 10, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 7 and/or (ii) a VH_{CD3} comprising the amino acid sequence of SEQ ID NO: 11, and a VL_{CD3} comprising the amino acid sequence of SEQ ID NO: 8;
(i) a heavy chain variable region (VH_{CD3}) comprising a heavy chain complementarity determining region (HCDR) 1 of SEQ ID NO: 1, a HCDR 2 of SEQ ID NO: 2, and a HCDR 3 of SEQ ID NO: 3, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 7 and/or (ii) a VH_{CD3} comprising the amino acid sequence of SEQ ID NO: 4, and a VL_{CD3} comprising the amino acid sequence of SEQ ID NO: 8; or
(i) a heavy chain variable region (VH_{CD3}) comprising a heavy chain complementarity determining region (HCDR) 1 of SEQ ID NO: 1, a HCDR 2 of SEQ ID NO: 2, and a HCDR 3 of SEQ ID NO: 10, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 13 and/or (ii) a VH_{CD3} comprising the amino acid sequence of SEQ ID NO: 12, and a VL_{CD3} comprising the amino acid sequence of SEQ ID NO: 14;
(A) a second and a third antigen binding domain that bind to CD20, comprising (i) a heavy chain variable region (VH_{CD20}) comprising a heavy chain complementarity determining region (HCDR) 1 of SEQ ID NO: 15, a HCDR 2 of SEQ ID NO: 16, and a HCDR 3 of SEQ ID NO: 17, and a light chain variable region (VL_{CD20}) comprising a light chain complementarity determining region (LCDR) 1 of SEQ ID NO: 19, a LCDR 2 of SEQ ID NO: 20 and a LCDR 3 of SEQ ID NO: 21 and/or (ii) a VH_{CD20} comprising the amino acid sequence of SEQ ID NO: 18, and a VL_{CD20} comprising the amino acid sequence of SEQ ID NO: 22; and (B) an Fc domain composed of a first and a second subunit;
wherein the first, the second and the third antigen binding domain are each a Fab molecule, and (i) the first and the third antigen binding domain are each fused at the C-terminus of the Fab heavy chain to the N-terminus of the first or the second subunit of the Fc domain, and the second antigen binding domain is fused at the C-terminus of the Fab heavy chain to the N-terminus of the Fab heavy chain of the first antigen binding domain, or (ii) the second and the third antigen binding domain are each fused at the C-terminus of the Fab heavy chain to the N-terminus of the first or the second subunit of the Fc domain, and the first antigen binding domain is fused at the C-terminus of the Fab heavy chain to the N-terminus of the Fab heavy chain of the second antigen binding domain.

In an even more specific aspect, the (multispecific) antibody of the invention comprises
(A) a first antigen binding domain that binds to CD3, wherein the first antigen binding domain is a Fab molecule wherein the variable domains VL and VH of the Fab light chain and the Fab heavy chain are replaced by each other, and comprises
(i) a heavy chain variable region (VH_{CD3}) comprising a heavy chain complementarity determining region (HCDR) 1 of SEQ ID NO: 1, a HCDR 2 of SEQ ID NO: 9, and a HCDR 3 of SEQ ID NO: 10, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 7 and/or (ii) a VH_{CD3} comprising the amino acid sequence of SEQ ID NO: 11, and a VL_{CD3} comprising the amino acid sequence of SEQ ID NO: 8;
(i) a heavy chain variable region (VH_{CD3}) comprising a heavy chain complementarity determining region (HCDR) 1 of SEQ ID NO: 1, a HCDR 2 of SEQ ID NO: 2, and a HCDR 3 of SEQ ID NO: 3, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 7 and/or (ii) a VH_{CD3} comprising the amino acid sequence of SEQ ID NO: 4, and a VL_{CD3} comprising the amino acid sequence of SEQ ID NO: 8; or
(i) a heavy chain variable region (VH_{CD3}) comprising a heavy chain complementarity determining region (HCDR) 1 of SEQ ID NO: 1, a HCDR 2 of SEQ ID NO: 2, and a HCDR 3 of SEQ ID NO: 10, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 13 and/or (ii) a VH_{CD3} comprising the amino acid sequence of SEQ ID NO: 12, and a VL_{CD3} comprising the amino acid sequence of SEQ ID NO: 14;
(A) a second and optionally a third antigen binding domain that bind to CD20, wherein the second and third antigen binding domain are each a (conventional) Fab molecule and comprise (i) a heavy chain variable region (VH_{CD20}) comprising a heavy chain complementarity determining region (HCDR) 1 of SEQ ID NO: 15, a HCDR 2 of SEQ ID NO: 16, and a HCDR 3 of SEQ ID NO: 17, and a light chain variable region (VL_{CD20}) comprising a light chain complementarity determining region (LCDR) 1 of SEQ ID NO: 19, a LCDR 2 of SEQ ID NO: 20 and a LCDR 3 of SEQ ID NO: 21 and/or (ii) a VH_{CD20} comprising the amino acid sequence of SEQ ID NO: 18, and a VL_{CD20} comprising the amino acid sequence of SEQ ID NO: 22;
wherein in the constant domain CL of the second antigen binding domain and, where present, the third antigen binding domain the amino acid at position 124 is substituted independently by lysine (K), arginine (R) or histidine (H) (numbering according to Kabat) (in a preferred aspect by lysine (K)) and the amino acid at position 123 is substituted independently by lysine (K), arginine (R) or histidine (H) (numbering according to Kabat) (in a preferred aspect by lysine (K) or arginine (R)), and in the constant domain CH1 of the second antigen binding domain and, where present, the third antigen binding domain the amino acid at position 147 is substituted independently by glutamic acid (E) or aspartic acid (D) (numbering according to Kabat EU index) (in a preferred aspect by glutamic acid (E)) and the amino acid at position 213 is substituted independently by glutamic acid (E) or aspartic acid (D) (numbering according to Kabat EU index) (in a preferred aspect by glutamic acid (E)); and
(C) a human IgG1 Fc domain composed of a first and a second subunit, comprising (i) in the first subunit the amino acid substitutions S354C and T366W, and in the second subunit the amino acid substitutions Y349C, T366S, L368A and Y407V; (ii) in each of its subunits the amino acid substitutions L234A, L235A and P329G; and optionally (iii) in each of its subunits (a) the amino acid substitutions M252Y, S254T and T256E, (b) the amino acid substitutions M428L and N434, or (c) the amino acid substitutions M428L, N434A, Q438R and S440E (numberings according to Kabat EU index);
wherein (i) the first antigen binding domain and, where present, the third antigen binding domain is fused at the C-terminus of the Fab heavy chain to the N-terminus of the first or the second subunit of the Fc domain, and the second antigen binding domain is fused at the C-terminus of the Fab heavy chain to the N-terminus of the Fab heavy chain of the first antigen binding domain, or (ii) the second antigen binding domain and, where present, the third antigen binding domain is fused at the C-terminus of the Fab heavy chain to the N-terminus of the first or the second subunit of the Fc domain, and the first antigen binding domain is fused at the C-terminus of the Fab heavy chain to the N-terminus of the Fab heavy chain of the second antigen binding domain.

In an even more specific aspect, the (multispecific) antibody of the invention comprises (A) a first antigen binding domain that binds to CD3, wherein the first antigen binding domain is a Fab molecule wherein the variable domains VL and VH of the Fab light chain and the Fab heavy chain are replaced by each other, and comprises
(i) a heavy chain variable region (VH_{CD3}) comprising a heavy chain complementarity determining region (HCDR) 1 of SEQ ID NO: 1, a HCDR 2 of SEQ ID NO: 9, and a HCDR 3 of SEQ ID NO: 10, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 7 and/or (ii) a VH_{CD3} comprising the amino acid sequence of SEQ ID NO: 11, and a VL_{CD3} comprising the amino acid sequence of SEQ ID NO: 8;
(i) a heavy chain variable region (VH_{CD3}) comprising a heavy chain complementarity determining region (HCDR) 1 of SEQ ID NO: 1, a HCDR 2 of SEQ ID NO: 2, and a HCDR 3 of SEQ ID NO: 3, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 7 and/or (ii) a VH_{CD3} comprising the amino acid sequence of SEQ ID NO: 4, and a VL_{CD3} comprising the amino acid sequence of SEQ ID NO: 8; or
(i) a heavy chain variable region (VH_{CD3}) comprising a heavy chain complementarity determining region (HCDR) 1 of SEQ ID NO: 1, a HCDR 2 of SEQ ID NO: 2, and a HCDR 3 of SEQ ID NO: 10, and a light chain variable region (VL_{CD3}) comprising a light chain complementarity determining region (LCDR) 1 of SEQ ID NO: 5, a LCDR 2 of SEQ ID NO: 6 and a LCDR 3 of SEQ ID NO: 13 and/or (ii) a VH_{CD3} comprising the amino acid sequence of SEQ ID NO: 12, and a VL_{CD3} comprising the amino acid sequence of SEQ ID NO: 14;
(A) a second and a third antigen binding domain that bind to CD20, wherein the second and third antigen binding domain are each a (conventional) Fab molecule and comprise (i) a heavy chain variable region (VH_{CD20}) comprising a heavy chain complementarity determining region (HCDR) 1 of SEQ ID NO: 15, a HCDR 2 of SEQ ID NO: 16, and a HCDR 3 of SEQ ID NO: 17, and a light chain variable region (VL_{CD20}) comprising a light chain complementarity determining region (LCDR) 1 of SEQ ID NO: 19, a LCDR 2 of SEQ ID NO: 20 and a LCDR 3 of SEQ ID NO: 21 and/or (ii) a VH_{CD20} comprising the amino acid sequence of SEQ ID NO: 18, and a VL_{CD20} comprising the amino acid sequence of SEQ ID NO: 22;
wherein in the constant domain CL of the second antigen binding domain and, where present, the third antigen binding domain the amino acid at position 124 is substituted by lysine (K) and the amino acid at position 123 is substituted by lysine (K) or arginine (R) (numbering according to Kabat), and in the constant domain CH1 of the second antigen binding domain and, where present, the third antigen binding domain the amino acid at position 147 is substituted by glutamic acid (E) (numbering according to Kabat EU index) and the amino acid at position 213 is substituted by glutamic acid (E) (numbering according to Kabat EU index); and
(C) a human IgG1 Fc domain composed of a first and a second subunit, comprising (i) in the first subunit the amino acid substitutions S354C and T366W, and in the second subunit the amino acid substitutions Y349C, T366S, L368A and Y407V; and (ii) in each of its subunits the amino acid substitutions L234A, L235A and P329G (numberings according to Kabat EU index);
wherein the first antigen binding domain and the third antigen binding domain are fused at the C-terminus of the Fab heavy chain to the N-terminus of the first or the second subunit of the Fc domain, and the second antigen binding domain is fused at the C-terminus of the Fab heavy chain to the N-terminus of the Fab heavy chain of the first antigen binding domain.

The invention also provides a (multispecific) antibody that binds to CD3 and to CD20, comprising a first heavy chain (HC1), a first light chain (LC1), a second heavy chain (HC2) and/or a second light chain (LC2), as described in the following.

In one aspect, the (multispecific) antibody of the invention comprises a first heavy chain (HC1) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 23 or SEQ ID NO: 24. In one aspect, the (multispecific) antibody comprises a HC1 comprising an amino acid sequence having at least 95% sequence identity to the amino acid sequence of SEQ ID NO: 23 or SEQ ID NO: 24. In one aspect, the (multispecific) antibody comprises a HC1 comprising an amino acid sequence having at least 98% sequence identity to the amino acid sequence of SEQ ID NO: 23 or SEQ ID NO: 24. In one aspect, the (multispecific) antibody comprises a HC1 comprising the amino acid sequence of SEQ ID NO: 23 or SEQ ID NO: 24 comprising up to two (i.e. 0, 1 or 2) amino acid substitutions within said amino acid sequence. In one aspect, the (multispecific) antibody comprises a HC1 comprising the amino acid sequence of SEQ ID NO: 23 or SEQ ID NO: 24 comprising one amino acid substitution within said amino acid sequence. In one aspect, the (multispecific) antibody comprises a HC1 comprising the amino acid sequence of SEQ ID NO: 23 or SEQ ID NO: 24 comprising two amino acid substitutions within said amino acid sequence. In one aspect, the (multispecific) antibody comprises a HC1 comprising the amino acid sequence of SEQ ID NO: 23 or SEQ ID NO: 24.

In one aspect, the (multispecific) antibody of the invention comprises a second heavy chain (HC2) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 25. In one aspect, the (multispecific) antibody comprises a HC2 comprising an amino acid sequence having at least 95% sequence identity to the amino acid sequence of SEQ ID NO: 25. In one aspect, the (multispecific) antibody comprises a HC2 comprising an amino acid sequence having at least 98% sequence identity to the amino acid sequence of SEQ ID NO: 25. In one aspect, the (multispecific) antibody comprises a HC2 comprising the amino acid sequence of SEQ ID NO: 25 comprising up to two (i.e. 0, 1 or 2) amino acid substitutions within said amino acid sequence. In one aspect, the (multispecific) antibody comprises a HC2 comprising the amino acid sequence of SEQ ID NO: 25 comprising one amino acid substitution within said amino acid sequence. In one aspect, the (multispecific) antibody comprises a HC2 comprising the amino acid sequence of SEQ ID NO: 25 comprising two amino acid substitutions within said amino acid sequence. In one aspect, the (multispecific) antibody comprises a HC2 comprising the amino acid sequence of SEQ ID NO: 25.

In one aspect, the (multispecific) antibody of the invention comprises a first light chain (LC1) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 27, SEQ ID NO: 28 or SEQ ID NO: 29. In one aspect, the (multispecific) antibody comprises a LC1 comprising an amino acid sequence having at least 95% sequence identity to the amino acid sequence of SEQ ID NO: 27, SEQ ID NO: 28 or SEQ ID NO: 29. In one aspect, the (multispecific) antibody comprises a LC1 comprising an amino acid sequence having at least 98% sequence identity to the amino acid sequence of SEQ ID NO: 27, SEQ ID NO: 28 or SEQ ID NO: 29. In one aspect, the (multispecific) antibody comprises a LC1 comprising the amino acid sequence of SEQ ID NO: 27, SEQ ID NO: 28 or SEQ ID NO: 29 comprising up to two (i.e. 0, 1 or 2) amino acid substitutions within said amino acid sequence. In one aspect, the (multispecific) antibody comprises a LC1 comprising the amino acid sequence of SEQ ID NO: 27, SEQ ID NO: 28 or SEQ ID NO: 29 comprising one amino acid substitution within said amino acid sequence. In one aspect, the (multispecific) antibody comprises a LC1 comprising the amino acid sequence of SEQ ID NO: 27, SEQ ID NO: 28 or SEQ ID NO: 29 comprising two amino acid substitutions within said amino acid sequence. In one aspect, the (multispecific) antibody comprises a LC1 comprising the amino acid sequence of SEQ ID NO: 27, SEQ ID NO: 28 or SEQ ID NO: 29.

In one aspect, the (multispecific) antibody of the invention comprises a second light chain (LC2) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 26. In one aspect, the (multispecific) antibody comprises a LC2 comprising an amino acid sequence having at least 95% sequence identity to the amino acid sequence of SEQ ID NO: 26. In one aspect, the (multispecific) antibody comprises a LC2 comprising an amino acid sequence having at least 98% sequence identity to the amino acid sequence of SEQ ID NO: 26. In one aspect, the (multispecific) antibody comprises a LC2 comprising the amino acid sequence of SEQ ID NO: 26 comprising up to two (i.e. 0, 1 or 2) amino acid substitutions within said amino acid sequence. In one aspect, the (multispecific) antibody comprises a LC2 comprising the amino acid sequence of SEQ ID NO: 26 comprising one amino acid substitution within said amino acid sequence. In one aspect, the (multispecific) antibody comprises a LC2 comprising the amino acid sequence of SEQ ID NO: 26 comprising two amino acid substitutions within said amino acid sequence. In one aspect, the (multispecific) antibody comprises a LC2 comprising the amino acid sequence of SEQ ID NO: 26.

In particular aspects, the (multispecific) antibody of the invention comprises a HC1 as in any of the aspects provided above, a HC2 as in any of the aspects provided above, a LC1 as in any of the aspects provided above, and a LC2 as in any of the aspects provided above. Preferably, the HC1 of SEQ ID NO: 23 (or a variation thereof, as described above) is combined with the LC1 of SEQ ID NO: 27 (or a variation thereof, as described above) or SEQ ID NO: 28 (or a variation thereof, as described above), and the HC1 of SEQ ID NO: 24 (or a variation thereof, as described above) is combined with the LC1 of SEQ ID NO: 29 (or a variation thereof, as described above). In aspects wherein the (multispecific) antibody comprises two antigen binding domains that bind to CD20 (e.g. wherein the antibody comprises the HC2 of SEQ ID NO: 25), the antibody comprises two LC2.

In a particular aspect, the (multispecific) antibody of the invention comprises a HC1 comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 23, a LC1 comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 28, a HC2 comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 25, and a LC2 comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 26. In one aspect, the (multispecific) antibody comprises a HC1 comprising the amino acid sequence of SEQ ID NO: 23 comprising up to two (i.e. 0, 1 or 2) amino acid substitutions within said amino acid sequence, a LC1 comprising the amino acid sequence of SEQ ID NO: 28 comprising up to two (i.e. 0, 1 or 2) amino acid substitutions within said amino acid sequence, a HC2 comprising the amino acid sequence of SEQ ID NO: 25 comprising up to two (i.e. 0, 1 or 2) amino acid substitutions within said amino acid sequence, and a LC2 comprising the amino acid sequence of SEQ ID NO: 26 comprising up to two (i.e. 0, 1 or 2) amino acid substitutions within said amino acid sequence. In one aspect, the (multispecific) antibody comprises a HC1 comprising the amino acid sequence of SEQ ID NO: 23, a LC1 comprising the amino acid sequence of SEQ ID NO: 28, a HC2 comprising the amino acid sequence of SEQ ID NO: 25, and a LC2 comprising the amino acid sequence of SEQ ID NO: 26.

In one aspect, the (multispecific) antibody of the invention comprises a HC1 comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 23, a LC1 comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 27, a HC2 comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 25, and a LC2 comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 26. In one aspect, the (multispecific) antibody comprises a HC1 comprising the amino acid sequence of SEQ ID NO: 23 comprising up to two (i.e. 0, 1 or 2) amino acid substitutions within said amino acid sequence, a LC1 comprising the amino acid sequence of SEQ ID NO: 27 comprising up to two (i.e. 0, 1 or 2) amino acid substitutions within said amino acid sequence, a HC2 comprising the amino acid sequence of SEQ ID NO: 25 comprising up to two (i.e. 0, 1 or 2) amino acid substitutions within said amino acid sequence, and a LC2 comprising the amino acid sequence of SEQ ID NO: 26 comprising up to two (i.e. 0, 1 or 2) amino acid substitutions within said amino acid sequence. In one aspect, the (multispecific) antibody comprises a HC1 comprising the amino acid sequence of SEQ ID NO: 23, a LC1 comprising the amino acid sequence of SEQ ID NO: 27, a HC2 comprising the amino acid sequence of SEQ ID NO: 25, and a LC2 comprising the amino acid sequence of SEQ ID NO: 26.

In one aspect, the (multispecific) antibody of the invention comprises a HC1 comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 24, a LC1 comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 29, a HC2 comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 25, and a LC2 comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 26. In one aspect, the (multispecific) antibody comprises a HC1 comprising the amino acid sequence of SEQ ID NO: 24 comprising up to two (i.e. 0, 1 or 2) amino acid substitutions within said amino acid sequence, a LC1 comprising the amino acid sequence of SEQ ID NO: 29 comprising up to two (i.e. 0, 1 or 2) amino acid substitutions within said amino acid sequence, a HC2 comprising the amino acid sequence of SEQ ID NO: 25 comprising up to two (i.e. 0, 1 or 2) amino acid substitutions within said amino acid sequence, and a LC2 comprising the amino acid sequence of SEQ ID NO: 26 comprising up to two (i.e. 0, 1 or 2) amino acid substitutions within said amino acid sequence. In one aspect, the (multispecific) antibody comprises a HC1 comprising the amino acid sequence of SEQ ID NO: 24, a LC1 comprising the amino acid sequence of SEQ ID NO: 29, a HC2 comprising the amino acid sequence of SEQ ID NO: 25, and a LC2 comprising the amino acid sequence of SEQ ID NO: 26.

In one aspect, the (multispecific) antibody of the invention comprises a HC1 comprising the amino acid sequence of SEQ ID NO: 23 or SEQ ID NO: 24 and additionally a C-terminal lysine residue (Lys447; Kabat EU numbering). In another aspect, the (multispecific) antibody comprises a HC1 comprising the amino acid sequence of SEQ ID NO: 23 or SEQ ID NO: 24 without the C-terminal glycine residue (Gly446; Kabat EU numbering). In such aspect, the C-terminal amino acid residue may be proline (Pro445; Kabat EU numbering) or proline amide (Pro445-NH₂; Kabat EU numbering).

In one aspect, the (multispecific) antibody of the invention comprises a HC2 comprising the amino acid sequence of SEQ ID NO: 25 and additionally a C-terminal lysine residue (Lys447; Kabat EU numbering). In another aspect, the (multispecific) antibody comprises a HC2 comprising the amino acid sequence of SEQ ID NO: 25 without the C-terminal glycine residue (Gly446; Kabat EU numbering). In such aspect, the C-terminal amino acid residue may be proline (Pro445; Kabat EU numbering) or proline amide (Pro445-NH₂; Kabat EU numbering).

### B. Polynucleotides

The invention further provides an isolated polynucleotide encoding an antibody of the invention. Said isolated polynucleotide may be a single polynucleotide or a plurality of polynucleotides.

The polynucleotides encoding a (multispecific) antibody of the invention may be expressed as a single polynucleotide that encodes the entire antibody or as multiple (e.g., two or more) polynucleotides that are co-expressed. Polypeptides encoded by polynucleotides that are co-expressed may associate through, e.g., disulfide bonds or other means to form a functional antibody. For example, the light chain portion of an antibody may be encoded by a separate polynucleotide from the portion of the antibody comprising the heavy chain of the antibody. When co-expressed, the heavy chain polypeptides will associate with the light chain polypeptides to form the antibody. In another example, the portion of the antibody comprising one of the two Fc domain subunits and optionally (part of) one or more Fab molecules could be encoded by a separate polynucleotide from the portion of the antibody comprising the other of the two Fc domain subunits and optionally (part of) a Fab molecule. When co-expressed, the Fc domain subunits will associate to form the Fc domain.

In one aspect, the isolated polynucleotide encodes the entire antibody molecule according to the invention as described herein. In another aspect, the isolated polynucleotide encodes a polypeptide comprised in the antibody according to the invention as described herein.

In one aspect, the polynucleotide is DNA. In another aspect, the polynucleotide is RNA, for example, in the form of messenger RNA (mRNA). RNA may be single stranded or double stranded.

### 1. Recombinant Methods and Compositions

(Multispecific) antibodies according to the invention may be produced using recombinant methods and compositions. For these methods one or more isolated polynucleotide(s) encoding the antibody are provided.

In one aspect, two polynucleotides are prepared, one for the light chain or a fragment thereof and one for the heavy chain or a fragment thereof. Such polynucleotide(s) encode an amino acid sequence comprising the VL and/or an amino acid sequence comprising the VH of the antibody (e.g., the light and/or heavy chain(s) of the antibody). These polynucleotides may be on the same expression vector or on different expression vectors.

In case of a multispecific (e.g. bispecific) antibody with heterodimeric heavy chains four polynucleotides are prepared, one for the first light chain, one for the first heavy chain comprising the first heteromonomeric Fc region polypeptide, one for the second light chain, and one for the second heavy chain comprising the second heteromonomeric Fc region polypeptide. The four polynucleotides may be comprised in one or more nucleic acid molecules or expression vectors. Such polynucleotide(s) encode (i) an amino acid sequence comprising the first VL (or, in the case of a crossover Fab molecule being comprised in the multispecific antibody, wherein the VH and VL are replaced by each other: the first VH) and/or (ii) an amino acid sequence comprising the first VH (or, in the case of a crossover Fab molecule being comprised in the multispecific antibody, wherein the VH and VL are replaced by each other: the first VL) including the first heteromonomeric Fc region and/or (iii) an amino acid sequence comprising the second VL and/or (iv) an amino acid sequence comprising the second VH including the second heteromonomeric Fc region of the antibody (e.g., the first and/or second light and/or the first and/or second heavy chains of the antibody). These polynucleotides can be on the same expression vector or on different expression vectors, normally these polynucleotides are located on two or three expression vectors, i.e. one vector can comprise more than one of these polynucleotides. In one aspect, isolated polynucleotides encoding an antibody as used in the methods as described herein are provided.

In one aspect, a method of making an antibody of the invention is provided, wherein the method comprises culturing a host cell comprising polynucleotide(s) encoding the antibody, as provided above, under conditions suitable for expression of the antibody, and optionally recovering the antibody from the host cell (including the host cell culture medium).

For recombinant production of a (multispecific) antibody, polynucleotides encoding the antibody, e.g., as described above, are prepared and inserted into one or more vectors for further cloning and/or expression in a host cell. Such polynucleotides may be readily isolated and sequenced using conventional procedures (e.g., by using oligonucleotide probes that are capable of binding specifically to genes encoding the heavy and light chains of the antibody) or produced by recombinant methods or obtained by chemical synthesis.

Suitable host cells for cloning or expression of antibody-encoding vectors include prokaryotic or eukaryotic cells as described herein. For example, antibodies may be produced in bacteria, in particular when glycosylation and Fc effector function are not needed. For expression of antibody fragments and polypeptides in bacteria, see, e.g., Charlton, K. A., In: Methods in Molecular Biology, Vol. 248, Lo, B. K. C. (ed.), Humana Press, Totowa, NJ, pp. 245-254 (2003), describing expression of antibody fragments in E. coli. After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized”, resulting in the production of an antibody with a partially or fully human glycosylation pattern. See Gerngross, T. U., Nat. Biotech. 22 1409-1414 (2004) and Li, H. et al., Nat. Biotech. 24: 210-215 (2006).

Suitable host cells for the expression of (glycosylated) antibodies are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing technology for producing antibodies in transgenic plants).

Vertebrate cells may also be used as hosts. For example, mammalian cell lines that are adapted to grow in suspension may be useful. Examples of useful mammalian host cell lines are monkey kidney CV1 line transformed by SV40 (COS-7); human embryonic kidney line (293 or 293T cells); baby hamster kidney cells (BHK); mouse sertoli cells (TM4 cells); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells; MRC 5 cells; and FS4 cells. Other useful mammalian host cell lines include Chinese hamster ovary (CHO) cells, including DHFR-CHO cells; and myeloma cell lines such as YO, NSO and Sp2/0.

For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, NJ, pp. 255-268 (2004).

In one aspect, the host cell is a eukaryotic cell, e.g., a Chinese Hamster Ovary (CHO) cell or a human embryonic kidney (HEK) cell.

In one aspect, the host cell is an isolated host cell. In one aspect, the host cell is not a cell within a human body.

When recombinant expression vectors encoding antibody genes are introduced into mammalian host cells, the antibodies are produced by culturing the host cells for a period of time sufficient to allow for expression of the antibody in the host cells or, in one aspect, secretion of the antibody into the culture medium in which the host cells are grown. Antibodies can be recovered and purified from recombinant cell cultures by well-known methods including, but not limited to, affinity chromatography (e.g. protein A chromatography), size exclusion chromatography, anion or cation exchange chromatography, mixed-mode chromatography, phosphocellulose chromatography, hydrophobic interaction chromatography, hydroxyapatite chromatography and lectin chromatography.

### C. Assays

A (multispecific) antibody provided herein may be identified, screened for, or characterized for its physical/chemical properties and/or biological activities by various assays known in the art.

### 1. Binding Assays

The binding (affinity) of the antibody to an Fc receptor or a target antigen can be determined for example by surface plasmon resonance (SPR), using standard instrumentation such as a BIAcore instrument (Cytiva), and receptors or target proteins such as may be obtained by recombinant expression. Alternatively, binding of antibodies to different receptors or target antigens may be evaluated using cell lines expressing the particular receptor or target antigen, for example by flow cytometry (FACS). A specific illustrative and exemplary aspect for measuring binding activity to CD3 is described in the following.

In one aspect, the binding affinity (K_{D}) to CD3 is determined by SPR as follows:

SPR is performed on a Biacore T200 instrument (Cytiva) at 25 °C. Biotinylated human CD3ε/δ (a heterodimer of CD3 delta and CD3 epsilon ectodomains fused to a human Fc domain with knob-into-hole modifications and a C-terminal Avi-tag; see SEQ ID NOs 38 and 39) is immobilized on a Neutravidin sensor chip (NAHLC200M, Xantec bioanalytics GmbH) with a surface density of approximately 50 resonance units (RU). As a running and dilution buffer, HEPES buffered saline (HBS) containing 0.05% Tween-20 is used. The anti-CD3 antibodies are injected onto the surface with a concentration series up to 300 nM for 90 s, and dissociation is monitored for 300 s. Subsequently, the surface is regenerated by injecting 10 mM glycine pH 2.0 for 60 s. Bulk refractive index differences are corrected by subtracting blank injections and by subtracting the response obtained from the reference flow cell without CD3ε/δ. Curve fitting is performed using the 1:1 Langmuir binding model within the Biacore evaluation software.

### 2. Activity Assays

Biological activity of a (multispecific) antibody of the invention can be measured by various assays as described in the Examples. Biological activities may for example include the induction of proliferation of T cells, the induction of signaling in T cells, the induction of expression of activation markers in T cells, the induction of cytokine secretion by T cells, the induction of lysis of target cells such as B-cells.

### D. Pharmaceutical Compositions

In a further aspect, provided are pharmaceutical compositions comprising the (multispecific) antibody of the invention, e.g., for use in any of the therapeutic methods described herein. In one aspect, a pharmaceutical composition comprises the (multispecific) antibody and a pharmaceutically acceptable carrier. In another aspect, a pharmaceutical composition comprises the (multispecific) antibody and at least one additional therapeutic agent, e.g., as described below.

Pharmaceutical compositions (formulations) of the (multispecific) antibody of the invention can be prepared by combining the antibody with pharmaceutically acceptable carriers or excipients known to the skilled person. See, for example Remington's Pharmaceutical Sciences 16th edition, Osol, A. Ed. (1980) and Falconer R. J., Biotechnology Advances 37: 107412 (2019). Exemplary pharmaceutical compositions of the antibody may be lyophilized, aqueous, frozen, etc. In one aspect, the pharmaceutical composition provided herein is for intravenous administration. In another aspect, the pharmaceutical composition is for subcutaneous administration.

Pharmaceutically acceptable carriers are generally non-toxic to recipients at the dosages and concentrations employed, and include, but are not limited to: buffers such as histidine, phosphate, citrate, acetate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

The pharmaceutical composition herein may also contain more than one active ingredient as necessary for the particular indication being treated, preferably those with complementary activities that do not adversely affect each other. For example, it may be desirable to further provide active ingredients indicated for the treatment of cancer or an autoimmune disease. Such active ingredients are suitably present in combination in amounts that are effective for the purpose intended.

The pharmaceutical compositions to be used for in vivo administration are generally sterile. Sterility may be readily accomplished, e.g., by filtration through sterile filtration membranes.

### E. Therapeutic Methods and Routes of Administration

Any of the antibodies of the invention may be used in therapeutic methods. Antibodies of the invention may be used as immunotherapeutic agents, for example in the treatment of cancer or an autoimmune disease.

In one aspect, antibodies of the invention for use as a medicament are provided. In a further aspect, antibodies of the invention for use in treating a disease are provided. In one aspect, antibodies of the invention for use in a method of treatment are provided. In one aspect, the invention provides an antibody of the invention for use in the treatment of a disease in an individual in need thereof. In one aspect, the invention provides an antibody for use in a method of treating an individual having a disease comprising administering to the individual an effective amount of the antibody. In one aspect, the disease to be treated is a proliferative disorder. In one aspect, the disease is cancer, particularly a cancer expressing the second antigen that the (multispecific) antibody of the invention binds to. In one aspect, the method further comprises administering to the individual an effective amount of at least one additional therapeutic agent, e.g., an anti-cancer agent if the disease to be treated is cancer. In a further aspect, the invention provides an antibody of the invention for use in inducing lysis of a target cell, particularly a target cell expressing the second antigen that the (multispecific) antibody of the invention binds to. In one aspect, the invention provides an antibody of the invention for use in a method of inducing lysis of a target cell, particularly a target cell expressing the second antigen that the (multispecific) antibody of the invention binds to, in an individual comprising administering to the individual an effective amount of the antibody to induce lysis of a target cell.

In a further aspect, the invention provides for the use of an antibody of the invention in the manufacture or preparation of a medicament. In one aspect, the medicament is for the treatment of a disease in an individual in need thereof. In a further aspect, the medicament is for use in a method of treating a disease comprising administering to an individual having the disease an effective amount of the medicament. In one aspect, the disease to be treated is a proliferative disorder. In one aspect, the disease is cancer, particularly a cancer expressing the second antigen that the (multispecific) antibody of the invention binds to. In one aspect, the method further comprises administering to the individual an effective amount of at least one additional therapeutic agent, e.g., an anti-cancer agent if the disease to be treated is cancer. In a further aspect, the medicament is for inducing lysis of a target cell, particularly a target cell expressing the second antigen that the (multispecific) antibody of the invention binds to. In still a further aspect, the medicament is for use in a method of inducing lysis of a target cell, particularly a target cell expressing the second antigen that the (multispecific) antibody of the invention binds to, in an individual comprising administering to the individual an effective amount of the medicament to induce lysis of a target cell.

In a further aspect, the invention provides a medicament (adapted) for the treatment of a disease, comprising the antibody of the invention. In one aspect the medicament is (adapted) for the treatment of a disease in an individual in need thereof. In a further aspect, the medicament is (adapted) for use in a method of treating a disease comprising administering to an individual having the disease an effective amount of the medicament. In one aspect, the disease is a proliferative disorder. In one aspect, the disease is cancer, particularly a cancer expressing the second antigen that the (multispecific) antibody of the invention binds to. In one aspect, the method further comprises administering to the individual an effective amount of at least one additional therapeutic agent, e.g., an anti-cancer agent if the disease to be treated is cancer. In a further aspect, the medicament is for inducing lysis of a target cell, particularly a target cell expressing the second antigen that the (multispecific) antibody of the invention binds to. In still a further aspect, the medicament is for use in a method of inducing lysis of a target cell, particularly a target cell expressing the second antigen that the (multispecific) antibody of the invention binds to, in an individual comprising administering to the individual an effective amount of the medicament to induce lysis of a target cell.

In a further aspect, the invention provides a method for treating a disease. In one aspect, the method comprises administering to an individual having such disease an effective amount of an antibody of the invention. In one aspect, a composition is administered to said individual, comprising the antibody of the invention in a pharmaceutically acceptable form. In one aspect, the disease is a proliferative disorder. In certain aspects the disease is cancer, particularly a cancer expressing the second antigen that the (multispecific) antibody of the invention binds to. In one aspect, the method further comprises administering to the individual an effective amount of at least one additional therapeutic agent, e.g., an anti-cancer agent if the disease to be treated is cancer. In a further aspect, the invention provides a method for inducing lysis of a target cell, particularly a target cell expressing the second antigen that the (multispecific) antibody of the invention binds to. In one aspect, the method comprises contacting a target cell with an antibody of the invention in the presence of a T cell, particularly a cytotoxic T cell. In a further aspect, a method for inducing lysis of a target cell, particularly a target cell expressing the second antigen that the (multispecific) antibody of the invention binds to, in an individual is provided. In one such aspect, the method comprises administering to the individual an effective amount of an antibody of the invention to induce lysis of a target cell.

In one aspect, the disease to be treated is a proliferative disorder, particularly cancer. Non-limiting examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. Other cell proliferation disorders that may be treated using an antibody of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases.

According to any of the above aspects, in particular wherein the antibody is a multispecific antibody binding to CD20 as the second antigen, the disease may be cancer.

In one such aspect, the cancer is a CD20-expressing cancer. By “CD20-positive cancer” or “CD20-expressing cancer” is meant a cancer characterized by expression or overexpression of CD20 in cancer cells. The expression of CD20 may be determined for example by quantitative real-time PCR (measuring CD20 mRNA levels), flow cytometry, immunohistochemistry (IHC) or western blot assays. In one aspect, the cancer expresses CD20. In one aspect, the cancer expresses CD20 in at least 20%, preferably at least 50% or at least 80% of tumor cells as determined by immunohistochemistry (IHC) using an antibody specific for CD20.

In a further such aspect, the cancer is a B-cell cancer, particularly a CD20-positive B-cell cancer.

In one aspect, the cancer is selected from the group consisting of Non-Hodgkin lymphoma (NHL), acute lymphocytic leukemia (ALL), chronic lymphocytic leukemia (CLL), diffuse large B-cell lymphoma (DLBCL), follicular lymphoma (FL), mantle-cell lymphoma (MCL), marginal zone lymphoma (MZL), Multiple myeloma (MM) or Hodgkin lymphoma (HL). In a particular aspect, the cancer is selected from the group consisting of Non-Hodgkin lymphoma (NHL), acute lymphocytic leukemia (ALL), chronic lymphocytic leukemia (CLL), diffuse large B-cell lymphoma (DLBCL), follicular lymphoma (FL), mantle-cell lymphoma (MCL) and marginal zone lymphoma (MZL). In a more particular aspect, the cancer is NHL, particularly relapsed/refractory (r/r) NHL. In one aspect, the cancer is DLBCL. In one aspect, the cancer is FL. In one aspect, the cancer is MCL. In one aspect, the cancer is MZL. Other cancers, particularly B-cell cancers, are also contemplated herein.

According to any of the above aspects, in particular wherein the antibody is a multispecific antibody binding to CD20 as the second antigen, the disease may be an autoimmune disease.

In one such aspect, the autoimmune disease is systemic lupus erythematosus (SLE), lupus nephritis (LN), rheumatoid arthritis (RA), multiple sclerosis (MS), type-1 diabetes mellitus, Graves' disease, inflammatory bowel disease (IBD) or Sjorgen's syndrome (SjS). Other autoimmune diseases, particularly B-cell mediated autoimmune diseases, are also contemplated herein.

An “individual” according to any of the above aspects is preferably a human. The individual according to any of the above aspects may be in need of the medicament and/or treatment with the antibody of the invention.

In a further aspect, the invention provides pharmaceutical compositions comprising an antibody of the invention, e.g., for use in any of the above therapeutic methods. In one aspect, a pharmaceutical composition comprises an antibody of the invention and a pharmaceutically acceptable carrier. In another aspect, a pharmaceutical composition comprises an antibody of the invention and at least one additional therapeutic agent, e.g., as described below.

Antibodies provided herein can be administered alone or used in a combination therapy. For instance, the combination therapy includes administering an antibody of the invention and administering at least one additional therapeutic agent (e.g. one, two, three, four, five, or six additional therapeutic agents). In one aspect, the combination therapy comprises administering an antibody of the invention and administering at least one additional therapeutic agent, such as a further anti-cancer agent.

Such combination therapies noted above encompass combined administration (where two or more therapeutic agents are included in the same or separate pharmaceutical composition(s)), and separate administration, in which case administration of the antibody of the invention can occur prior to, simultaneously, and/or following, administration of the additional therapeutic agent or agents. In one aspect, administration of the antibody and administration of an additional therapeutic agent occur within about one, two, three, four, five, or six days, within about one, two or three weeks, or within about one month, of each other. In one aspect, the antibody and additional therapeutic agent are administered to the patient on Day 1 of the treatment. Antibodies of the invention can also be used in combination with radiation therapy.

An antibody of the invention (and any additional therapeutic agent) can be administered by any suitable means, including parenteral, intrapulmonary, intranasal and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies of the invention would be formulated, dosed, and administered in a fashion consistent with good medical practice. Factors for consideration in this context include the particular disorder being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to treat the disorder in question. The effective amount of such other agents depends on the amount of antibody present in the pharmaceutical composition, the type of disorder or treatment, and other factors discussed above. These are generally used in the same dosages and with administration routes as described herein, or from about 1 to 99% of the dosages described herein, or in any dosage and by any route that is empirically/clinically determined to be appropriate.

For the treatment of disease, the appropriate dosage of an antibody of the invention (when used alone or in combination with one or more additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. For repeated administrations over several days or longer, depending on the condition, the treatment would generally be sustained until a desired suppression of disease symptoms occurs. The progress of this therapy is easily monitored by conventional techniques and assays.

### F. Articles of Manufacture

In another aspect of the invention, an article of manufacture containing materials useful for the treatment, prevention and/or diagnosis of the disorders described above is provided. The article of manufacture comprises a container and a label or package insert on or associated with the container.

Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this aspect of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### G. Methods and Compositions for Diagnostics and Detection

In one aspect, any of the antibodies provided herein is useful for detecting the presence of its target (e.g. CD3) in a biological sample. The term “detecting” as used herein encompasses quantitative or qualitative detection. In one aspect, a biological sample comprises a cell or tissue, such as cancer tissue.

In one aspect, an antibody according to the invention for use in a method of diagnosis or detection is provided. In a further aspect, a method of detecting the presence of CD3 in a biological sample is provided. In one aspect, the method comprises contacting the biological sample with an antibody of the invention under conditions permissive for binding of the antibody to CD3, and detecting whether a complex is formed between the antibody and CD3. Such method may be an in vitro or in vivo method. In one aspect, an antibody of the invention is used to select subjects eligible for therapy with an antibody that binds CD3, e.g. where CD3 is a biomarker for selection of patients.

Exemplary disorders that may be diagnosed using an antibody of the invention include cancer.

In one aspect, an antibody according to the present invention is provided, wherein the antibody is labelled. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction.

III.
SEQUENCES

| | | SEQ ID |
|---|---|---|
| | Amino acid sequence | NO |
| CD3 HCDR1 | SYAMN | 1 |
| CD3 HCDR2 | RQRTQYNNREDYYADSVKG | 2 |
| CD3 HCDR3 | HSNFPSSYDSYFQY | 3 |
| CD3 VH | EVQLLESGGGLVQPGGSLRLSCAASGFTFSSYAMNWVRQAPGKGLEWVGRQRTQYN | 4 |
| | NREDYYADSVKGRFTISRDDSKNTLYLQMNSLRAEDTAVYYCVRHSNFPSSYDSYF | |
| | QYWGQGTLVTVSS | |
| CD3 LCDR1 | GSSTGAVTTSNYAN | 5 |
| CD3 LCDR2 | GTNKLAP | 6 |
| CD3 LCDR3 | ALWYSNLWY | 7 |
| CD3 VL | QAVVTQEPSLTVSPGGTVTLTCGSSTGAVTTSNYANWVQEKPGQPPRGLIGGTNKL | 8 |
| | APGTPARFSGSLLGGKAALTLSGAQPEDEAEYYCALWYSNLWYFGGGTKLTVL | |
| CD3 HCDR2 | RQRTQYHDRADYYADSVKG | 9 |
| CD3 HCDR3 | HSNFPTSYDSYFEY | 10 |
| CD3 VH | EVQLLESGGGLVQPGGSLRLSCAASGFTFSSYAMNWVRQAPGKGLEWVGRQRTQYH | 11 |
| | DRADYYADSVKGRFTISRDDSKNTLYLQMNSLRAEDTAVYYCVRHSNFPTSYDSYF | |
| | EYWGQGTLVTVSS | |
| CD3 VH | EVQLLESGGGLVQPGGSLRLSCAASGFTFSSYAMNWVRQAPGKGLEWVGRQRTQYN | 12 |
| | NREDYYADSVKGRFTISRDDSKNTLYLQMNSLRAEDTAVYYCVRHSNFPTSYDSYF | |
| | EYWGQGTLVTVSS | |
| CD3 LCDR3 | ALWYSNLWV | 13 |
| CD3 VL | QAVVTQEPSLTVSPGGTVTLTCGSSTGAVTTSNYANWVQEKPGQPPRGLIGGTNKL | 14 |
| | APGTPARFSGSLLGGKAALTLSGAQPEDEAEYYCALWYSNLWVFGGGTKLTVL | |
| CD20 HCDR1 | YSWIN | 15 |
| CD20 HCDR2 | RIFPGDGDTDYNGKFKG | 16 |
| CD20 HCDR3 | NVFDGYWLVY | 17 |
| CD20 VH | QVQLVQSGAEVKKPGSSVKVSCKASGYAFSYSWINWVRQAPGQGLEWMGRIFPGDG | 18 |
| | DTDYNGKFKGRVTITADKSTSTAYMELSSLRSEDTAVYYCARNVEDGYWLVYWGQG | |
| | TLVTVSS | |
| CD20 LCDR1 | RSSKSLLHSNGITYLY | 19 |
| CD20 LCDR2 | QMSNLVS | 20 |
| CD20 LCDR3 | AQNLELPYT | 21 |
| CD20 VL | DIVMTQTPLSLPVTPGEPASISCRSSKSLLHSNGITYLYWYLQKPGQSPQLLIYQM | 22 |
| | SNLVSGVPDRFSGSGSGTDFTLKISRVEAEDVGVYYCAQNLELPYTFGGGTKVEIK | |
| CD20 (VH- | QVQLVQSGAEVKKPGSSVKVSCKASGYAFSYSWINWVRQAPGQGLEWMGRIFPGDG | 23 |
| CH1)-CD3 | DTDYNGKFKGRVTITADKSTSTAYMELSSLRSEDTAVYYCARNVFDGYWLVYWGQG | |
| (VL-CH1)-Fc | TLVTVSSASTKGPSVFPLAPSSKSTSGGTAALGCLVEDYFPEPVTVSWNSGALTSG | |
| (knob) | VHTFPAVLQSSGLYSLSSVVTVPSSSLGTQTYICNVNHKPSNTKVDEKVEPKSCGG | |
| | GGSGGGGQAVVTQEPSLTVSPGGTVTLTCGSSTGAVTTSNYANWVQEKPGQPPRGL | |
| | IGGTNKLAPGTPARFSGSLLGGKAALTLSGAQPEDEAEYYCALWYSNLWYFGGGTK | |
| | LTVLSSASTKGPSVFPLAPSSKSTSGGTAALGCLVKDYFPEPVTVSWNSGALTSGV | |
| | HTFPAVLQSSGLYSLSSVVTVPSSSLGTQTYICNVNHKPSNTKVDKKVEPKSCDKT | |
| | HTCPPCPAPEAAGGPSVFLFPPKPKDTLMISRTPEVTCVVVDVSHEDPEVKFNWYV | |
| | DGVEVHNAKTKPREEQYNSTYRVVSVLTVLHQDWLNGKEYKCKVSNKALGAPIEKT | |
| | ISKAKGQPREPQVYTLPPCRDELTKNQVSLWCLVKGFYPSDIAVEWESNGQPENNY | |
| | KTTPPVLDSDGSFFLYSKLTVDKSRWQQGNVFSCSVMHEALHNHYTQKSLSLSPG | |
| CD20 (VH- | QVQLVQSGAEVKKPGSSVKVSCKASGYAFSYSWINWVRQAPGQGLEWMGRIFPGDG | 24 |
| CH1)-CD3 | DTDYNGKFKGRVTITADKSTSTAYMELSSLRSEDTAVYYCARNVFDGYWLVYWGQG | |
| (VL-CH1)-Fc | TLVTVSSASTKGPSVFPLAPSSKSTSGGTAALGCLVEDYFPEPVTVSWNSGALTSG | |
| (knob) | VHTFPAVLQSSGLYSLSSVVTVPSSSLGTQTYICNVNHKPSNTKVDEKVEPKSCGG | |
| | GGSGGGGQAVVTQEPSLTVSPGGTVTLTCGSSTGAVTTSNYANWVQEKPGQPPRGL | |
| | IGGTNKLAPGTPARFSGSLLGGKAALTLSGAQPEDEAEYYCALWYSNLWVEGGGTK | |
| | LTVLSSASTKGPSVFPLAPSSKSTSGGTAALGCLVKDYFPEPVTVSWNSGALTSGV | |
| | HTFPAVLQSSGLYSLSSVVTVPSSSLGTQTYICNVNHKPSNTKVDKKVEPKSCDKT | |
| | HTCPPCPAPEAAGGPSVFLFPPKPKDTLMISRTPEVTCVVVDVSHEDPEVKFNWYV | |
| | DGVEVHNAKTKPREEQYNSTYRVVSVLTVLHQDWLNGKEYKCKVSNKALGAPIEKT | |
| | ISKAKGQPREPQVYTLPPCRDELTKNQVSLWCLVKGFYPSDIAVEWESNGQPENNY | |
| | KTTPPVLDSDGSFFLYSKLTVDKSRWQQGNVFSCSVMHEALHNHYTQKSLSLSPG | |
| CD20 (VH- | QVQLVQSGAEVKKPGSSVKVSCKASGYAFSYSWINWVRQAPGQGLEWMGRIFPGDG | 25 |
| CH1)-Fc | DTDYNGKFKGRVTITADKSTSTAYMELSSLRSEDTAVYYCARNVFDGYWLVYWGQG | |
| (hole) | TLVTVSSASTKGPSVFPLAPSSKSTSGGTAALGCLVEDYFPEPVTVSWNSGALTSG | |
| | VHTFPAVLQSSGLYSLSSVVTVPSSSLGTQTYICNVNHKPSNTKVDEKVEPKSCDK | |
| | THTCPPCPAPEAAGGPSVFLFPPKPKDTLMISRTPEVTCVVVDVSHEDPEVKFNWY | |
| | VDGVEVHNAKTKPREEQYNSTYRVVSVLTVLHQDWLNGKEYKCKVSNKALGAPIEK | |
| | TISKAKGQPREPQVCTLPPSRDELTKNQVSLSCAVKGFYPSDIAVEWESNGQPENN | |
| | YKTTPPVLDSDGSFFLVSKLTVDKSRWQQGNVFSCSVMHEALHNHYTQKSLSLSPG | |
| CD20 (VL-CL) | DIVMTQTPLSLPVTPGEPASISCRSSKSLLHSNGITYLYWYLQKPGQSPQLLIYQM | 26 |
| | SNLVSGVPDRFSGSGSGTDFTLKISRVEAEDVGVYYCAQNLELPYTFGGGTKVEIK | |
| | RTVAAPSVFIFPPSDRKLKSGTASVVCLLNNFYPREAKVQWKVDNALQSGNSQESV | |
| | TEQDSKDSTYSLSSTLTLSKADYEKHKVYACEVTHQGLSSPVTKSFNRGEC | |
| CD3 (VH-CL) | EVQLLESGGGLVQPGGSLRLSCAASGFTFSSYAMNWVRQAPGKGLEWVGRQRTQYN | 27 |
| | NREDYYADSVKGRFTISRDDSKNTLYLQMNSLRAEDTAVYYCVRHSNFPSSYDSYF | |
| | QYWGQGTLVTVSSASVAAPSVFIFPPSDEQLKSGTASVVCLLNNFYPREAKVQWKV | |
| | DNALQSGNSQESVTEQDSKDSTYSLSSTLTLSKADYEKHKVYACEVTHQGLSSPVT | |
| | KSFNRGEC | |
| CD3 (VH-CL) | EVQLLESGGGLVQPGGSLRLSCAASGFTFSSYAMNWVRQAPGKGLEWVGRQRTQYH | 28 |
| | DRADYYADSVKGRFTISRDDSKNTLYLQMNSLRAEDTAVYYCVRHSNFPTSYDSYF | |
| | EYWGQGTLVTVSSASVAAPSVFIFPPSDEQLKSGTASVVCLLNNFYPREAKVQWKV | |
| | DNALQSGNSQESVTEQDSKDSTYSLSSTLTLSKADYEKHKVYACEVTHQGLSSPVT | |
| | KSFNRGEC | |
| CD3 (VH-CL) | EVQLLESGGGLVQPGGSLRLSCAASGFTFSSYAMNWVRQAPGKGLEWVGRQRTQYN | 29 |
| | NREDYYADSVKGRFTISRDDSKNTLYLQMNSLRAEDTAVYYCVRHSNFPTSYDSYF | |
| | EYWGQGTLVTVSSASVAAPSVFIFPPSDEQLKSGTASVVCLLNNFYPREAKVQWKV | |
| | DNALQSGNSQESVTEQDSKDSTYSLSSTLTLSKADYEKHKVYACEVTHQGLSSPVT | |
| | KSFNRGEC | |
| Human CD3 | QDGNEEMGGITQTPYKVSISGTTVILTCPQYPGSEILWQHNDKNIGGDEDDKNIGS | 30 |
| | DEDHLSLKEFSELEQSGYYVCYPRGSKPEDANFYLYLRARVCENCMEMDVMSVATI | |
| | VIVDICITGGLLLLVYYWSKNRKAKAKPVTRGAGAGGRQRGQNKERPPPVPNPDYE | |
| | PIRKGQRDLYSGLNQRRI | |
| Cynomolgus | QDGNEEMGSITQTPYQVSISGTTVILTCSQHLGSEAQWQHNGKNKEDSGDRLELPE | 31 |
| CD3 | FSEMEQSGYYVCYPRGSNPEDASHHLYLKARVCENCMEMDVMAVATIVIVDICITL | |
| | GLLLLVYYWSKNRKAKAKPVTRGAGAGGRQRGQNKERPPPVPNPDYEPIRKGQQDL | |
| | YSGLNQRRI | |
| Human | MTTPRNSVNGTFPAEPMKGPIAMQSGPKPLERRMSSLVGPTQSFFMRESKTLGAVQ | 32 |
| CD20 | IMNGLFHIALGGLLMIPAGIYAPICVTVWYPLWGGIMYIISGSLLAATEKNSRKCL | |
| | VKGKMIMNSLSLFAAISGMILSIMDILNIKISHFLKMESLNFIRAHTPYINIYNCE | |
| | PANPSEKNSPSTQYCYSIQSLFLGILSVMLIFAFFQELVIAGIVENEWKRTCSRPK | |
| | SNIVLLSAEEKKEQTIEIKEEVVGLTETSSQPKNEEDIEIIPIQEEEEEETETNEP | |
| | EPPQDQESSPIENDSSP | |
| hIgG₁ Fc | DKTHTCPPCPAPELLGGPSVFLFPPKPKDTLMISRTPEVTCVVVDVSHEDPEVKEN | 33 |
| region | WYVDGVEVHNAKTKPREEQYNSTYRVVSVLTVLHQDWLNGKEYKCKVSNKALPAPI | |
| | EKTISKAKGQPREPQVYTLPPSRDELTKNQVSLTCLVKGFYPSDIAVEWESNGQPE | |
| | NNYKTTPPVLDSDGSFFLYSKLTVDKSRWQQGNVFSCSVMHEALHNHYTQKSLSLS | |
| | P | |
| Human | RTVAAPSVFIFPPSDEQLKSGTASVVCLLNNFYPREAKVQWKVDNALQSGNSQESV | 34 |
| kappa CL | TEQDSKDSTYSLSSTLTLSKADYEKHKVYACEVTHQGLSSPVTKSFNRGEC | |
| domain | | |
| Human | QPKAAPSVTLEPPSSEELQANKATLVCLISDFYPGAVTVAWKADSSPVKAGVETTT | 35 |
| lambda CL | PSKQSNNKYAASSYLSLTPEQWKSHRSYSCQVTHEGSTVEKTVAPTECS | |
| domain | | |
| Human IgG₁ | ASTKGPSVFPLAPSSKSTSGGTAALGCLVKDYFPEPVTVSWNSGALTSGVHTFPAV | 36 |
| heavy chain | LQSSGLYSLSSVVTVPSSSLGTQTYICNVNHKPSNTKVDKKVEPKSCDKTHTCPPC | |
| constant | PAPELLGGPSVFLFPPKPKDTLMISRTPEVTCVVVDVSHEDPEVKENWYVDGVEVH | |
| region (CH1- | NAKTKPREEQYNSTYRVVSVLTVLHQDWLNGKEYKCKVSNKALPAPIEKTISKAKG | |
| CH2-CH3) | QPREPQVYTLPPSRDELTKNQVSLTCLVKGFYPSDIAVEWESNGQPENNYKTTPPV | |
| | LDSDGSFFLYSKLTVDKSRWQQGNVFSCSVMHEALHNHYTQKSLSLSP | |
| Linker | GGGGSGGGG | 37 |
| Human CD3 | QDGNEEMGGITQTPYKVSISGTTVILTCPQYPGSEILWQHNDKNIGGDEDDKNIGS | 38 |
| epsilon stalk- | DEDHLSLKEFSELEQSGYYVCYPRGSKPEDANFYLYLRARVSENCVDEQLYFQGGS | |
| Fc(knob)- | PKSADKTHTCPPCPAPELLGGPSVFLFPPKPKDTLMISRTPEVTCVVVDVSHEDPE | |
| Avi | VKFNWYVDGVEVHNAKTKPREEQYNSTYRVVSVLTVLHQDWLNGKEYKCKVSNKAL | |
| | PAPIEKTISKAKGQPREPQVYTLPPCRDELTKNQVSLWCLVKGFYPSDIAVEWESN | |
| | GQPENNYKTTPPVLDSDGSFFLYSKLTVDKSRWQQGNVFSCSVMHEALHNHYTQKS | |
| | LSLSPGKSGGLNDIFEAQKIEWHE | |
| Human CD3 | FKIPIEELEDRVFVNCNTSITWVEGTVGTLLSDITRLDLGKRILDPRGIYRCNGTD | 39 |
| delta stalk- | IYKDKESTVQVHYRMCRSEQLYFQGDKTHTCPPCPAPELLGGPSVFLFPPKPKDTL | |
| Fc (hole)- | MISRTPEVTCVVVDVSHEDPEVKFNWYVDGVEVHNAKTKPREEQYNSTYRVVSVLT | |
| Avi | VLHQDWLNGKEYKCKVSNKALPAPIEKTISKAKGQPREPQVCTLPPSRDELTKNQV | |
| | SLSCAVKGFYPSDIAVEWESNGQPENNYKTTPPVLDSDGSFFLVSKLTVDKSRWQQ | |
| | GNVFSCSVMHEALHNHYTQKSLSLSPGKSGGLNDIFEAQKIEWHE | |
| CD3 HCDR2 | RIRSKYNNYATYYADSVKG | 40 |
| CD3_{orig} | | |
| CD3 HCDR3 | ASNFPASYVSYFAY | 41 |
| CD3_{orig} | | |
| CD3 VH | EVQLLESGGGLVQPGGSLRLSCAASGFTFSSYAMNWVRQAPGKGLEWVSRIRSKYN | 42 |
| CD3orig | NYATYYADSVKGRFTISRDDSKNTLYLQMNSLRAEDTAVYYCVRASNFPASYVSYF | |
| | AYWGQGTLVTVSS | |
| CD3 LCDR2 | GTNKRAP | 43 |
| CD3_{orig} | | |
| CD3 VL | QAVVTQEPSLTVSPGGTVTLTCGSSTGAVTTSNYANWVQEKPGQAFRGLIGGTNKR | 44 |
| CD3_{orig} | APGTPARFSGSLLGGKAALTLSGAQPEDEAEYYCALWYSNLWVFGGGTKLTVL | |

### IV. Examples

The following are examples of methods and compositions of the invention. It is understood that various other aspects may be practiced, given the general description provided above.

### Example 1—Preparation of Optimized Anti-CD3 (Multispecific) Antibodies

Starting from the previously described anti-CD3 antibody CD3_{orig} (comprising the VH and VL sequences of SEQ ID NOs 42 and 44, respectively; see WO 2021/255142, incorporated herein by reference in its entirety) as a parental CD3 binder backbone, we generated optimized CD3 binders.

For the generation of optimized CD3 binder variants, we used structure-based calculations to identify positively-charged and hydrophobic patches (MOE, Discovery Studio) and sequence-based predictions (NetMHCllpan 4.0) to predict potential T-cell epitopes. Besides the parental binder CD3_{orig}, we analyzed the data of more than 1000 different CD3 binder variants tested before. For the subsequent design rounds, the approach revolved around substituting solvent-exposed and non-antigen binding variable region amino acids containing positively charged or hydrophobic side chains by residues with preferably negatively-charged or polar side chains, while constraining the sequence space such that the number of predicted MHC-II binding peptides in the designed sequences was minimized.

We focused on phenotypes with beneficial specificity, reduced immunogenicity risk, reduced hydrophobic or positive patchiness, and moderate affinity, while maintaining other desirable features such as stability, humanness etc. Our goal in engineering increased specificity was, besides other considerations, the reduction of unwanted off-target side-effects. By reducing positive patchiness, we aimed for reduced nonspecific uptake/presentation of the antibody in addition to potentially increased half-life. The reduction of strong potential T-cell epitopes was done to yield less recognition of MHC II presented peptides by T-cells and potentially immunogenic reactions caused by the antibody which might result in anti-drug antibody (ADA) formation in patients. By reducing affinity of CD3_{orig}, we aimed to reach a sweet spot of potency and cytokine release.

We underwent three consecutive design/make/test cycles to generate a large number of >100 one-armed IgG anti-CD3 antibodies by combinatorial protein engineering methods known in the art. The clones were subjected to a screening cascade for productivity, purity, and binding. Interesting clones were analyzed further by biochemical and in vitro methods. After two initial screening rounds with >100 variants, from a pool of 35 third-round molecules, we selected three CD3 variants that fulfilled our criteria best, FV043862, FV043861 and FV032417, with binding affinities (K_{D}) to CD3 of 160 nM, 243 nM and 302 nM, respectively.

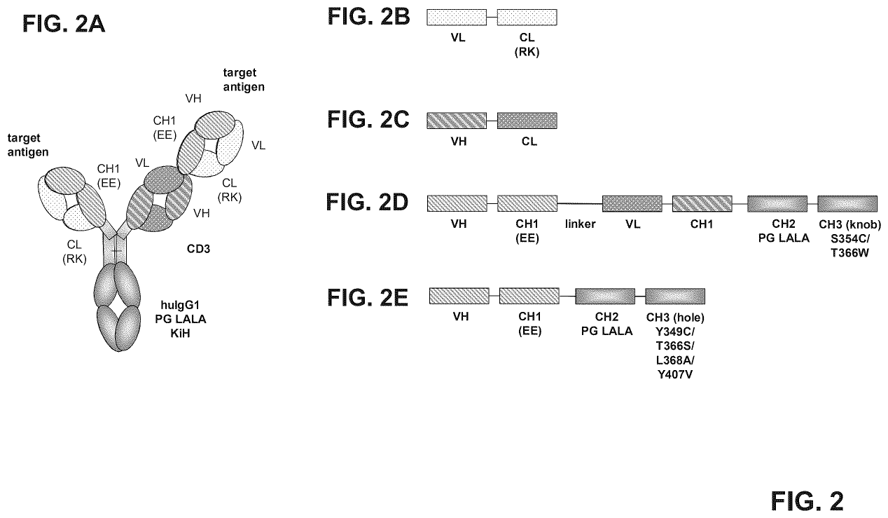

For direct comparison, the binders were produced as T-cell bispecific antibodies (TCBs) in the “2+1” format as depicted in FIG. 2, using an anti-CD20 antibody as target cell antigen binding domain (SEQ ID NOs 15-22). The variable regions of heavy and light chain DNA sequences were subcloned in frame with either the constant heavy chain or the constant light chain pre-inserted into the respective recipient mammalian expression vectors as shown in FIG. 2B-2E.

Sequences of the selected optimized anti-CD3 antibodies are given in the SEQ ID NOs indicated in Table 1.

TABLE 1
Sequences (SEQ ID NOs) of selected optimized anti-CD3 antibodies generated
in the present Examples, as well as the parental antibody CD3_{orig}

| | HCDR1 | HCDR2 | HCDR3 | VH | LCDR1 | LCDR2 | LCDR3 | VL |
|---|---|---|---|---|---|---|---|---|
| CD3_{orig} | 1 | 40 | 41 | 42 | 5 | 43 | 13 | 44 |
| FV043862 (160 nM) | 1 | 2 | 3 | 4 | 5 | 6 | 7 | 8 |
| FV043861 (243 nM) | 1 | 9 | 10 | 11 | 5 | 6 | 7 | 8 |
| FV032417 (302 nM) | 1 | 2 | 10 | 12 | 5 | 6 | 13 | 14 |

To improve correct pairing of the light chains with the corresponding heavy chains, mutations were introduced in the human CL (E123R, Q124K (Kabat numbering)) and the human CH1 (K147E, K213E (EU numbering)) of the CD20 binding Fab molecule.

For correct pairing of the heavy chains (formation of a heterodimeric molecule), knob-into-hole mutations were introduced in the constant region of the antibody heavy chains (T366W/S354C and T366S/L368A/Y407V/Y349C (EU numbering), respectively).

Furthermore, the P329G, L234A and L235A (EU numbering) mutations were introduced in the constant region of the antibody heavy chains to abrogate binding to Fcγ receptors.

Full sequences of the prepared TCB molecules are given in the SEQ ID NOs indicated in Table 2. A corresponding molecule comprising CD3_{orig} as CD3 binder, and an anti-CD19 antibody as target cell antigen binding domain, was also prepared (P1AM6465).

TABLE 2
Sequences of TCB molecules generated in the present Examples

| | HC | HC | LC1 | LC2 |
|---|---|---|---|---|
| | (knob)* | (hole)* | (CD3) | (CD20) |
| P1AN0288 = 2 + 1 TCB with | 23 | 25 | 27 | 26 |
| FV043862 (160 nM) | | | | |
| P1AN0289 = 2 + 1 TCB with | 23 | 25 | 28 | 26 |
| FV043861 (243 nM) | | | | |
| P1AM3067 = 2 + 1 TCB with | 24 | 25 | 29 | 26 |
| FV032417 (302 nM) | | | | |

*Fc with KiH and PGLALA

Antibodies were produced by transient transfection of Expi293F cells. Cells were seeded in Expi293 media (Gibco, #1435101) at a density of 2.5×10⁶/ml. Expression vectors and ExpiFectamine (Gibco, ExpiFectamine transfection kit, #13385544) were separately mixed in OptiMEM (Gibco, #11520386). After 5 minutes both solutions were combined, mixed by pipetting and incubated for 15-20 minutes at room temperature. Cells were added to the vector/ExpiFectamine solution and incubated for 24 hours at 37 °C in a shaking incubator with a 5% CO₂ atmosphere. One day post transfection, supplements (Enhancer 1+2, ExpiFectamine transfection kit) were added. Cell supernatants were harvested after 4-5 days by centrifugation and subsequent filtration (0.2 μm filter), and proteins were purified from the harvested supernatant by standard methods as indicated below. Proteins were purified from filtered cell culture supernatants referring to standard protocols. In brief, Fc containing proteins were purified from cell culture supernatants by Protein A-affinity chromatography (equilibration buffer: 20 mM sodium citrate, 20 mM sodium phosphate, pH 7.5; elution buffer: 20 mM sodium citrate, pH 3.0). Elution was achieved at pH 3.0 followed by immediate pH neutralization of the sample. The protein was concentrated by centrifugation (Millipore Amicon® ULTRA-15 (#UFC903096), and aggregated protein was separated from monomeric protein by size exclusion chromatography in 20 mM histidine, 140 mM sodium chloride, pH 6.0.

The concentrations of purified proteins were determined by measuring the absorption at 280 nm using the mass extinction coefficient calculated on the basis of the amino acid sequence according to Pace et al., Protein Science 4, 2411-1423 (1995). Purity and molecular weight of the proteins were analyzed by CE-SDS in the presence and absence of a reducing agent using a LabChipGXII (Perkin Elmer). Determination of the aggregate content was performed by HPLC chromatography at 25 °C using analytical size-exclusion column (TSKgel G3000 SW XL or UP-SW3000) equilibrated in running buffer (25 mM K₂HPO₄, 125 mM NaCl, 200 mM L-arginine monohydrocloride, pH 6.7 or 200 mM KH₂PO₄, 250 mM KCl pH 6.2, respectively).

All antibodies could be produced in good quality.

### Example 2—Determination of Hydrophobicity, Heparin Binding and Thermal Stability of Optimized Anti-CD3 (Multispecific) Antibodies

Hydrophobicity of the optimized anti-CD3 antibodies (FV043862, FV043861 and FV032417) was assessed by hydrophobic interaction chromatography using a TSKgel Ether-5PW column (Tosoh Bioscience). Samples were eluted using a salt gradient from 1.5 M ammonium sulfate to 0 M in a 20 mM sodium phosphate buffer pH 7.0. Relative retention times were calculated using an internal reference standard IgG.

Heparin binding was assessed by affinity chromatography using a TSKgel Heparin-5PW column (Tosoh Bioscience). Samples were eluted using a salt gradient from 0 to 1 M NaCl in a 50 mM Tris buffer pH 7.4. Relative retention times were calculated using an internal reference standard IgG.

Results are shown in Table 3 (including for the control molecule P1AM6465).

TABLE 3
Hydrophobic interaction chromatography (HIC) and binding to
a heparin column with retention times relative to an internal
IgG standard of anti-CD3 antibodies (in TCB format)

| TCB | HIC | Heparin |
|---|---|---|
| P1AM3067 | 0.36 | 0.52 |
| P1AN0288 | 0.36 | 0.52 |
| P1AN0289 | 0.36 | 0.53 |
| P1AM6465 | 0.22 | 0.78 |
| (CD3_{orig}) | | |

While the hydrophobicity according to the HIC profile of the optimized anti-CD3 antibodies remains similar to the parental clone, heparin binding is reduced and therefore general unspecific binding may be less likely.

Thermal stability was assessed by static light scattering using an UNCLE instrument (Unchained Labs). The aggregation propensity of the antibodies was monitored during heating up from 30 °C to 90 °C, the onset temperature of aggregation was determined at 266 nm.

Results are shown in Table 4 (including the control molecule P1AM6465).

TABLE 4
Thermal stability of anti-CD3 antibodies (in TCB
format) as measured by static light scattering

| TCB | T_{agg} [°C] |
|---|---|
| P1AM3067 | 58.5 |
| P1AN0288 | 56.3 |
| P1AN0289 | 55.6 |
| P1AM6465 | 61.7 |
| (CD3_{orig}) | |

The engineered variants show comparable, or slightly reduced but still acceptable, heat stability as compared to CD3_{orig}.

### Example 3—Characterization of Optimized Anti-CD3 (Multispecific) Antibodies by Surface Plasmon Resonance (SPR)

Binding affinities and kinetics of the optimized anti-CD3 antibodies were investigated using a Biacore T200 instrument (Cytiva) at 25 °C. Biotinylated human CD3E/6 (CD3E stalk-Fc(knob)-Avi/CD36 stalk-Fc(hole), see SEQ ID NOs 38 and 39) was immobilized on a Neutravidin sensor chip (NAHLC200M, Xantec bioanalytics GmbH) with a surface density of approximately 50 resonance units (RU). As a running and dilution buffer, HEPES buffered saline (HBS) containing 0.05% Tween-20 was used. The anti-CD3 antibodies were injected onto the surface with a concentration series up to 300 nM for 90 s, dissociation was monitored for 300 s. Subsequently, the surface was regenerated by injecting 10 mM glycine pH 2.0 for 60 s. Bulk refractive index differences were corrected by subtracting blank injections and by subtracting the response obtained from the reference flow cell without CD3E/. Curve fitting was performed using the 1:1 Langmuir binding model within the Biacore evaluation software.

Results are shown in Table 5.

TABLE 5
Affinity of anti-CD3 antibodies (in TCB format) to human CD3ε/δ

| TCB | ka (1/Ms) | kd (1/s) | t½ (s) | K_{D} (nM) |
|---|---|---|---|---|
| P1AM3067 | 1.57E+05 | 4.72E−02 | 15 | 302 |
| P1AN0288 | 3.91E+05 | 6.26E−02 | 11 | 160 |
| P1AN0289 | 3.18E+05 | 7.73E−02 | 9 | 243 |
| P1AM6465 | 2.61E+06 | 1.42E−03 | 488 | 0.5 |
| (CD3_{orig}) | | | | |

The selected variants show a lower affinity to CD3 as compared to the parental binder CD3_{orig}(P1AM6465). This lower affinity was aimed for as described above and further shown below.

### Example 4—in Silico Prediction of MHC-II Binding Non-Weak Cores of Optimized Anti-CD3 Antibodies

We used NetMHCIIpan 4.0 in order to predict the number of MHC-II binding peptides in the variable regions (VH and VL) of the optimized anti-CD3 antibodies. For this, we predicted binding to the nine common HLA-DRB1 alleles HLA-DRB1*01:01, HLA-DRB1*03:01, HLA-DRB1*04:01, HLA-DRB1*0701, HLA-DRB1*08:01, HLA-DRB1*09:01, HLA-DRB1*11:01, HLA-DRB1*13:01, HLA-DRB1*15:01, with a “strong binder” threshold of Percentile Rank 2, and a “weak binder” threshold of Percentile Rank 10. In order to characterize the sequence-related immunogenicity risk, we counted the number of unique 9-mers that are predicted to bind to at least one of the tested MHC-II alleles in the strong Percentile Rank range (% Rank >=0 and % Rank <=2), or to four or more of the tested HLA-DRB1 alleles in the weak Percentile rank range (% Rank >2 and % Rank <=10). We denote this quantity as the number of non-weak cores (#NWC). The results are shown in Table 6. For reference, the raw predictor output, i.e., the number of 15-mer peptides in the respective Percentile Rank ranges as identified by NetMHCIIpan 4.0 is also given (#strong, #weak, #total).

TABLE 6
MHC-Il binding scores predicted by NetMHCIIpan

| Binder | #NWC | #strong | #weak | #total |
|---|---|---|---|---|
| CD3_{orig} | 12 | 30 | 173 | 203 |
| FV032417 | 5 | 8 | 107 | 115 |
| FV043862 | 5 | 8 | 108 | 116 |
| FV043861 | 5 | 7 | 107 | 114 |

In order to subtract MHC-II binding peptides with a high probability of being non-antigenic, we did not count peptides that are occurring in 10 or more human V region germlines. Hence, the above table only counts peptides which are either non-germline, or are occurring only in a very small number of human germlines.

The data shows that the three optimized anti-CD3 antibodies are predicted to have a reduced number of MHC-II-binding 9-mer cores when compared to the sequence of CD3_{orig}, which should lead to less peptide-MHC-II presentation on cells, and a lower potential risk for sequence-related immunogenicity.

### Example 5—Hydrophobic and Charged-Patch Volume of Optimized Anti-CD3 Antibodies Calculated from X-Ray Structure and Homology Models

One focus of the rational design attempt was to reduce the size of hydrophobic and positively charged patches in the variable region of the anti-CD3 antibodies in order to reduce the rate of non-specific uptake by cells and in general to improve the biophysical properties of the molecules.

Using homology models of the Fv region with capped C-termini, protonation states at pH 7.4 were assigned with PROPKA (an in silico method for the prediction of the pKa values of ionizable residues in proteins; Bas et al., Proteins: Structure, Function, and Bioinformatics, 73(3), 765-783 (2008)), and the 3D charge distribution was calculated with APBS (Adaptive Poisson-Boltzmann Solver software, solving the equations of continuum electrostatics for biomolecules and used to study intra- and intermolecular electrostatic interactions; Jurrus et al., Protein Science 27(1), 112-128 (2018)) at 150 mM ionic strength. The volume integral of all positive and negative voxels is reported as the patch score in Table 7.

TABLE 7
Positive and negative charged patch volume of anti-CD3 antibodies
(volume integral of all positive and negative voxels)

| | Integral_positive | | Integral_negative | |
|---|---|---|---|---|
| Binder | Absolute | Percent Change | Absolute | Percent Change |
| CD3_{orig} | 46060 | n/a | −8589 | n/a |
| FV032417 | 22455 | −51.25% | −12490 | +45.42% |
| FV043862 | 27675 | −39.92% | −11773 | +37.07% |
| FV043861 | 23267 | −49.49% | −12893 | +50.11% |

For the three optimized anti-CD3 antibodies, the size of the positive charge volume integral decreases by up to almost 52%, while the size of the negative charge volume integral increases (by approximately 37 to 50%).

The in silico measure for hydrophobicity was calculated with a method that is an implementation of Spatial Aggregation Propensity (SAP) (Chennamsetty et al., PNAS 106, 11937-11942 (2009)) using the Wimley-White hydrophobicity scale (Wimley and White, Nat Struct Mol Biol 3, 842-848 (1996)). The results are listed in Table 8. Lower values indicate less hydrophobic character.

TABLE 8
Hydrophobicity values of anti-CD3 antibodies

Surface Hydrophobicity (SAP)

| | Absolute | Percent Change |
|---|---|---|
| CD3_{orig} | 125.82 | n/a |
| FV032417 | 110.84 | −11.91% |
| FV043862 | 110.37 | −12.28% |
| FV043861 | 101.44 | −19.38% |

For the three optimized anti-CD3 antibodies, the calculated hydrophobicity value decreases by approximately 12 to 20%.

### Example 6—Polyreactivity Testing of Optimized Anti-CD3 (Multispecific) Antibodies

Polyreactivity of the optimized anti-CD3 antibodies was assessed using an ELISA based setup with 13 different poly-antigens coated to a maxisorp 96 well plate (ThermoFisher Scientific) at 1 μg/ml each overnight at 4 °C. After a washing and blocking step using phosphate buffered saline (with 2% bovine serum albumine for blocking), samples were incubated for 1 h at room temperature. After washing the plate once more, bound antibodies were detected using an anti-human IgG horseradish peroxidase (HRP) labeled antibody (JacksonImmunoResearch). The optical density was determined using tetramethylbenzidine (TMB) peroxidase substrate at a wavelength of 370 and 492 nm.

Results are shown in Table 9.

TABLE 9
Polyreactivity score of anti-CD3 antibodies (in TCB format).
The number indicates the count of poly-antigens, which are
bound within a set of antigens used in an ELISA-based setup

| | Polyreactivity |
|---|---|
| TCB | score |
| P1AM6465 | 0 |
| (CD3_{orig}) | |
| P1AM3067 | 0 |
| P1AN0288 | 0 |
| P1AN0289 | 0 |

The new variants show no binding to non-target compounds in the biochemical assay.

### Example 7—Non-Specific Cell Binding of Optimized Anti-CD3 (Multispecific) Antibodies

An ELISA was performed to evaluate the “stickiness” of the optimized anti-CD3 antibodies by measuring the specific and unspecific binding on cells.

This ELISA was established to detect unspecific binding of anti-CD3 antibodies of different formats to adherent cells. Therefore, adherent cells without CD3 expression on their surface (e.g. CHO-K1) were used and binding was measured. Cells were seeded over night in growth medium, antibodies were added and incubated for 2 h at room temperature. The cells were carefully washed with PBS, fixed with glutaraldehyde, and washed again with PBS/2% FBS. Bound antibodies were detected using a F(ab)2 fragment goat <human Fcγ fragment>POD (peroxidase), incubation at room temperature. After washing steps with PBS/2% FBS, the substrate TMB (3,3′,5,5′ tetramethylbenzidine) was added and the enzymatic reaction was stopped after 10 min using H₂SO₄. The amounts of bound antibodies were measured by detecting the absorbances in correlation to the antibody concentrations at 450 nm with a Tecan Infinite reader.

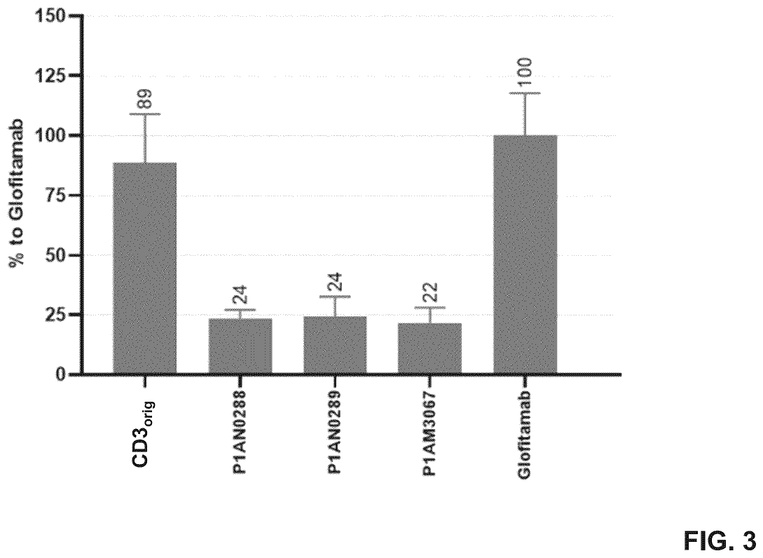

The result (for an antibody concentration of 200 nM) is shown in FIG. 3. The signal correlated to the non-specific binding to target (i.e. CD3)-negative cells. The optimized anti-CD3 antibodies FV032417, FV043862 and FV043861 (as CD20×CD3 TCBs P1AM3067, P1AN0288 and P1AN0289, respectively) show reduced stickiness in this experiment, with a signal (as compared to glofitamab, which was used as reference in this assay) of 22%, 24% and 24%, respectively, while the parental CD3 binder CD3_{orig} showed a signal of 89% (CD3_{orig} was used as CD19×CD3 TCB, but it was confirmed in previous experiments that the difference in target cell binders does not have a relevant impact on the outcome of this assay).

### Example 8—In Vitro PK Parameters—Non-Specific Cellular Accumulation (LUCA)

Antibodies containing extensive positively charged patches on their surface, particularly in their Fv region, show elevated non-specific clearance in vivo (see e.g. Kraft et al., mAbs 12, 1683432 (2020), Liu et al., mAbs 13, 1993769 (2021)). Measurement of cellular accumulation rates using the LUCA assay in human primary liver sinusoidal endothelial cells can be used to estimate the non-specific clearance of therapeutic antibodies.

The LUCA assay makes use of a pH dependent difference in fluorescence intensity of a fluorophore covalently attached to the antibody construct of interest during the in vitro procedure. The construct with the attached dye is then incubated with human primary liver sinusoidal endothelial cells which express physiological levels of human FcRn. The fluorescence in the endothelial cell remains low if the labeled construct shows low endocytosis rates and is well recycled by the FcRn in the endothelial cell, but increases if the labeled construct shows high endocytosis-driven uptake and is not well recycled and is processed in late endosomes and degraded in the lysosome, since the pH is decreasing in these late endosomes and the lysosome. The higher the LUCA value, the higher may be the in vivo clearance.

Details to the approach can be found in PCT application WO 2021/204743 (incorporated herein by reference in its entirety).

Briefly, data is acquired by labeling the antigen binding molecules with a pH-sensitive dye, exhibiting high fluorescence when accumulating in the lysosome (acidic pH 5.5) and low fluorescence when remaining outside the cell (neutral pH 7.4). Human or animal endothelial cells are incubated with labeled antibodies for 2 and 4 hours and the fluorescent readout is recorded using a flow cytometer. The geo-mean intensities are used for linear regression analysis. The extracted slopes form, when normalized to standard antibodies, the so-called normalized non-specific cellular accumulation rate.

Antibody Labeling: The antibodies were labeled using the SiteClick™ Antibody Azido Modification Kit (Thermo Fisher Scientific) according to the manufacturer's instructions. Briefly, N-linked galactose residues of the Fc-region were removed by β3-galactosidase and replaced by an azide-containing galactose (GalNaz) via β3-1,4-galactosyltransferase (GalT). This azide modification enables a copper-free conjugation of sDIBO-modified dyes. The pH-sensitive amine-reactive dye (523 nm) was purchased from Promega and coupled to a sulfo DBCO PEG4 amine. Antibodies were labeled with a molar dye excess of 2. Excess dye was removed using the Amicon® Ultra-2 Centrifugal Filter with a MWCO of 50 kD (EMD Millipore, #UFC200324) and antibodies were re-buffered in 20 mM histidine buffer (pH 5.5). The concentration of the labeled antibodies [1] as well as the dye to antibody ratio (DAR) [2] was determined with a Nanodrop spectrometer at 280 nm and 532 nm.

CAB=[A280 nm−[A280 nm*CFDye]]/εmAb [1]

DAR=[A532 nm*MWmAb]/[cmAb*εDye] [2]
εDye=47225 15 CFDye=0.36

Cell Maintenance and Preparation: Cryopreserved human liver-derived endothelial cells (HLEC-P2) were purchased from Lonza (Lonza, #HLECP2). Cells were maintained in EBM Endothelial Cell Growth Basal Medium-2 (Lonza, #CC-3156) supplemented with EGM-2 MV (Lonza, #CC-4176). Five days prior antibody treatment, cells were plated onto collagen I coated 100 mm culture dishes (Corning® BioCoat™, #354450) and two days prior treatment sub-cultured into collagen I coated 96-well plates (Corning® BioCoat™, #354407) at a cell density of 4×10⁴. On the day of the experiment, cells were washed twice with 200 μl pre-warmed medium and subsequently incubated with 400 nM labeled antibody or 20 mM histidine buffer (pH 5.5) as negative control in medium. After 2 and 4 hours, the antibody solution was removed and cells were washed once with 200 μl ice-cold DPBS (without Mg and Ca) and detached by applying 100 μl trypsin (with EDTA) for 2.5 minutes at 37 °C. Trypsin was inactivated by the addition of 100 μl FACS Buffer (20% FCS, 1 mM EDTA in DPBS).

Quality control: Biophysical binding properties are key determinants affecting clearance mechanisms. Therefore, it was important to assess whether the binding affinities of the antibodies changed during the labeling process. Heparin chromatography and neonatal Fc receptor binding has been previously shown to predict antibody clearance in vitro (Kraft et al., mAbs 12, 1683432 (2020)).

Herein, this method was used to account for potential aberrant binding properties introduced by the click label. To confirm the absence of unbound dye and to verify the concentration measured at the spectrometer, a size exclusion chromatography of the labeled antibodies was performed. Samples were separated using a BioSuite Diol (OH) column (Waters, #186002165) with a potassium dihydrogen phosphate buffer (pH 6.2) as the mobile phase at a flow rate of 0.5 ml/min. Detectors at 280 nm and 532 nm were used to quantify and analyze the labeled antibodies. The area under the curve (AUCs) at 280 nm and 532 nm was extracted to calculate the concentration. The geo-mean of the AUCs from all antibodies was computed and the deviation from each antibody to this geo-mean was identified. For an antibody to be reliable within this assay, the difference from the geo-mean was expected to be below 15%.

Flow Cytometry and Pharmacokinetic Analysis: The mean fluorescent intensity (MFI, more specifically the geometric mean (geo-mean)) of the internalized antibodies was acquired using the MACSQuant® Analyzer 10 (Miltenyi Biotec) equipped with a laser to excite at 488 nm and a filter to collect emitted light at 585 nm/540 nm. The exact same conditions, gains and gates were used for both times points (2 hours and 4 hours). Data extraction was performed using the FloJo_V10 software. Values of the negative control were subtracted from all geo-mean values followed by normalization to the DAR. The normalized geo-mean values from each antibody were plotted as a linear regression curve using GraphPad Prism to extract the slope (Geo Mean MFI/min for 120 and 240 min). Two standard antibodies were selected to normalize the slopes:

Motavizumab-YTE (G. J. Robbie et al., Antimicrob. Agent Chemother. 2013, 57(12), 6147) was set to 0, and glofitamab was set to 1.

Testing of anti-CD3 antibodies: By reducing the size of positively charged patches in the variable region of CD3_{orig}, the successfully engineered CD3 binders should show superior pharmacokinetic properties including reduced non-specific clearance.

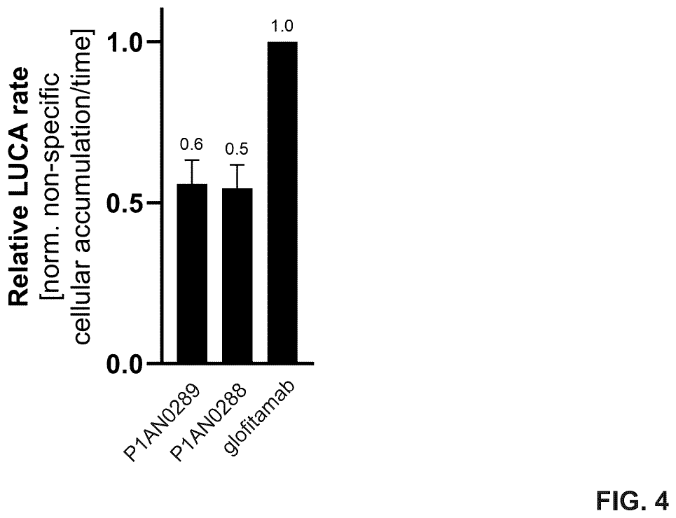

The optimized anti-CD3 antibodies were compared in “2+1” TCB format, where they are present monovalently in combination with a bivalent CD20 binder. Here, we could evaluate the effect of the positive patch removal. FIG. 4 shows a strongly reduced cellular accumulation rate for P1AN0288 and P1AN0289, as compared to glofitamab (P1AM3067 was not available for testing in this assay). This finding is further supported by the strong correlation between the results of this in vitro study and the percent change in volume integral of all positive voxels (Example 5, Table 7).

### Example 9—B-Cell Depletion and Cytokine (IL-6) Release with Broad Affinity Range of Anti-CD3 Antibodies as CD3×CD20 T-Cell Bispecific Antibodies (TCBs)

A set of anti-CD3 antibodies with a broad range of affinity to CD3 (Table 10) were assessed as CD3×CD20 T-cell bispecific antibodies (TCBs) for their potency to deplete B-cells and trigger IL-6 release in a 72 h in vitro assay. All TCBs were in the “2+1” format illustrated in FIG. 2, and comprised a CD20 binder with the VH and VL sequences of SEQ ID NOs 18 and 22, respectively.

For assessing the cytotoxic activity and cytokine secretion, a FACS-based B-cell killing assay was set up with a subsequent determination of cytokine concentrations in the cell culture supernatants.

Briefly, 200000 PBMCs per 96 well (U-bottom, Corning Costar, #3799) containing B-cells as target cells (TCs) and T-cells as effector cells (ECs) were cultured with the antibodies in triplicates (final concentration 100 nM-1×10⁻⁶ nM, dilution series 1:10, 9 dilutions) in assay medium (RPMI1640 (Gibco, #A1049101), 10% FCS (heat inactivated, Gibco, #10500-064), 1% Penicillin/Streptomycin (Gibco, #15140-172)). Untreated PBMCs were used as control. After 24 h incubation (37 °C, 5% CO₂, humidified atmosphere) the supernatants were taken and frozen at −80 °C for a subsequent cytokine analysis via a multiplexed Meso Scale Discovery U-Plex assay measuring IFN-γ, TNF-α, IL-6 and IL-2 concentrations.

The cell pellets of the PBMC assay were FACS-stained with a multiparameter panel (Table 11) determining B-cell counts (TCs), and T-cell (EC) counts and activation (CD25, CD69 expression). The count of CD19+ B cells was normalized to CountBright™ Absolute Counting Beads (Thermo Scientific, #C36950). The percentage of B-cell depletion was calculated as follows: B-cell depletion [%]=100−(100×B-cell count normalized to counting beads/normalized B-cell count in untreated PBMCs). The EC₅₀ value was calculated as the inflection point and the Eₘₐₓ value as the maximum of a four parameter logistic regression. Glofitamab served as a reference. To address donor variabilities, all values were normalized to those of glofitamab treated samples per donor and assay day.

TABLE 10
Anti-CD3 antibodies tested in this Example (as CD3 ×
CD20 TCBs) with CD3 binding values. The selected optimized
molecules discussed in the previous Examples are shown in bold

| | |
|---|---|
| CD3 binding | CD3 binding |

| CD3 × | t ½ | K_{D} | CD3 × | t ½ | K_{D} |
|---|---|---|---|---|---|
| CD20 TCB | [s] | [nM] | CD20 TCB | [s] | [nM] |
| Glofitamab | 230 | 5 | P1AN0288 | 11 | 160 |
| P1AN0291 | 20 | 117 | P1AN0289 | 9 | 243 |
| P1AN0286 | 16 | 92 | P1AM3067 | 15 | 302 |
| P1AN0287 | 15 | 150 | P1AN0290 | 3 | 586 |

TABLE 11
FACS staining panel

| Antibody | Vendor | Cat# |
|---|---|---|
| Brilliant Violet 421 anti-human CD45 | Biolegend | 304032 |
| Brilliant Violet 605 anti-human CD14 | Biolegend | 301834 |
| Brilliant Violet 605 anti-human CD56 | Biolegend | 318334 |
| (NCAM) | | |
| Brilliant Violet 605 anti-human CD15 | Biolegend | 323032 |
| (SSEA-1) | | |
| Brilliant Violet 605 anti-human CD16 | Biolegend | 302040 |
| BUV 395 anti-human CD40 | BD Bioscience | 565202 |
| Brilliant Blue 515 mouse anti-human | BD Bioscience | 564456 |
| CD19 | | |
| PE/Cyanine7 anti-human CD20 | Biolegend | 302312 |
| Brilliant Violet 785 anti-human CD4 | Biolegend | 344642 |
| Brilliant Violet 510 anti-human CD8 | Biolegend | 344732 |
| PerCP/Cyanine5.5 anti-human CD3 | Biolegend | 300430 |
| Alexa Fluor 647 anti-human CD25 | Biolegend | 356128 |
| PE anti-human CD69 | Biolegend | 310906 |

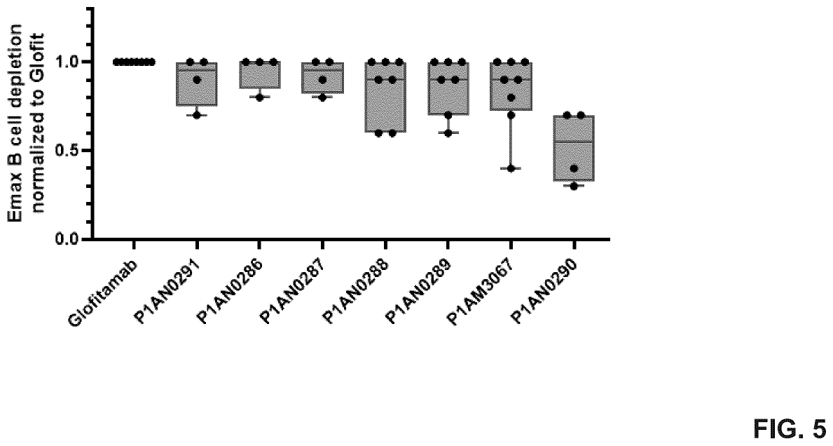

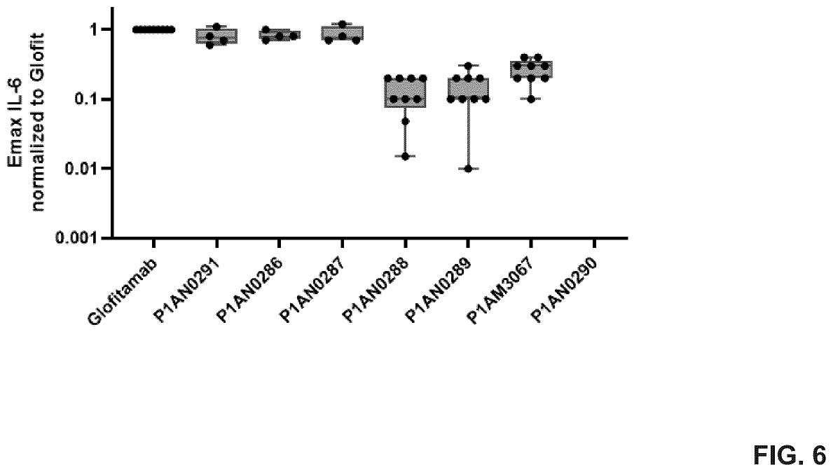

FIG. 5 and FIG. 6 summarize the Eₘₐₓ values of the B-cell depletion and IL-6 secretion, respectively, in the PBMC-based B-cell killing assay. To address donor variabilities all values were normalized to those of Glofitamab treated samples per donor and assay day. (Minimum to maximum with median). For the B-cell depletion, the median of the Eₘₐₓ stays even for compounds with affinities down to 370 nM at a high level of approx. 90% compared to glofitamab. The Eₘₐₓ only drops significantly for P1AN0290 with an affinity of 598 nM. Interestingly, the Eₘₐₓ of the IL-6 secretion decreases stepwise, staying at a level of >75% normalized to glofitamab up to an affinity of 150 nM and then dropping to 18-35% for affinities between 160-302 nM and being not calculable anymore for 598 nM. Taken together, these data identify an optimal window of CD3 binder affinities between 160 nM and 302 nM-encompassing the optimized anti-CD3 antibodies described herein-in which the level of B-cell depletion stays at a high level while the IL-6 secretion drops significantly.

### Example 10—B-Cell Depletion and Cytokine Release Properties of Optimized Anti-CD3 (Multispecific) Antibodies In Vitro Using Human Tonsil Derived Lymphocytes

The optimized anti-CD3 antibodies, as CD3×CD20 TCBs, were evaluated and benchmarked to glofitamab for their B-cell depletion potency and cytokine release in human tonsil derived lymphocyte cell cultures.

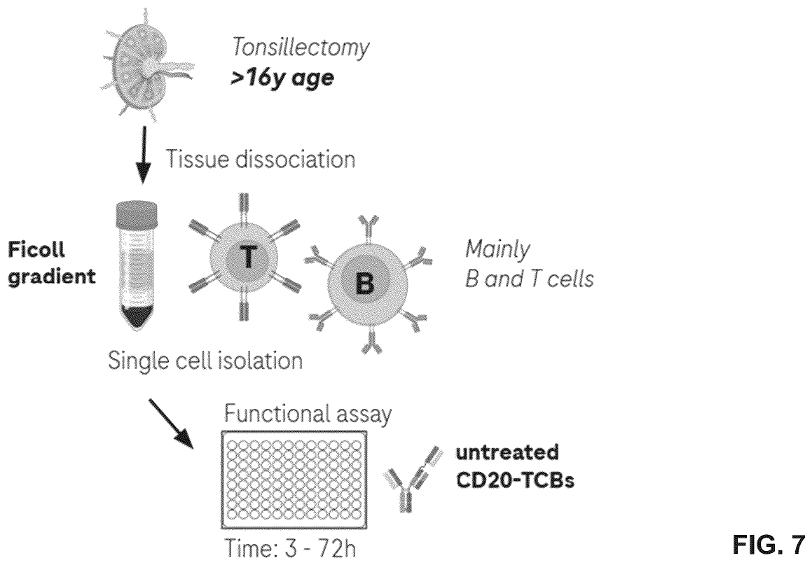

The collection of human tonsil tissue and the subsequent functional assay for testing the antibodies for B-cell depletion on tonsillar mononuclear cells (tMNC) was conducted as follows. In brief, human palatine tonsils were collected from >16 years old donors. Tonsillar mononuclear cells (tMNCs) were isolated by DNasel and collagenase D enzymatic digestion of tissue. The obtained cell suspension was filtered through a 100 μm cell strainer and tonsillar lymphocytes isolated though lymphoprep density gradient centrifugation in SepMate tubes. The isolated tMNCs were counted, resuspended in 10% FBS containing RPMI 1640 medium and cell suspension seeded per well of a 96-well U-bottom plate. Antibodies were diluted in PBS with 4 consecutive 10-fold dilutions. Each antibody dilution was added to wells containing cells, while PBS was added to vehicle controls. Cells were treated with P1AM3067, P1AN0288 and P1AN0289, as well as glofitamab, in a concentration range of 10 μM to 100 nM (5 concentrations, 10-fold dilutions). The setup of the assay is schematically depicted in FIG. 7.

Each concentration was tested in duplicate, while vehicle controls were tested in three replicas per plate. Cell cultures were incubated up to 72 h at 37 °C, 5% CO₂. At 24 h, cell culture supernatants were collected to determine cytokine concentration and at 72 h, cells were analyzed by flow cytometry. Cytokine measurement was performed using Invitrogen ProcartaPlex kit and Luminex 200 analyser, according to the manufacturer's instructions. The ZE5 instrument was used for multi-parameter flow cytometry sample acquisition.

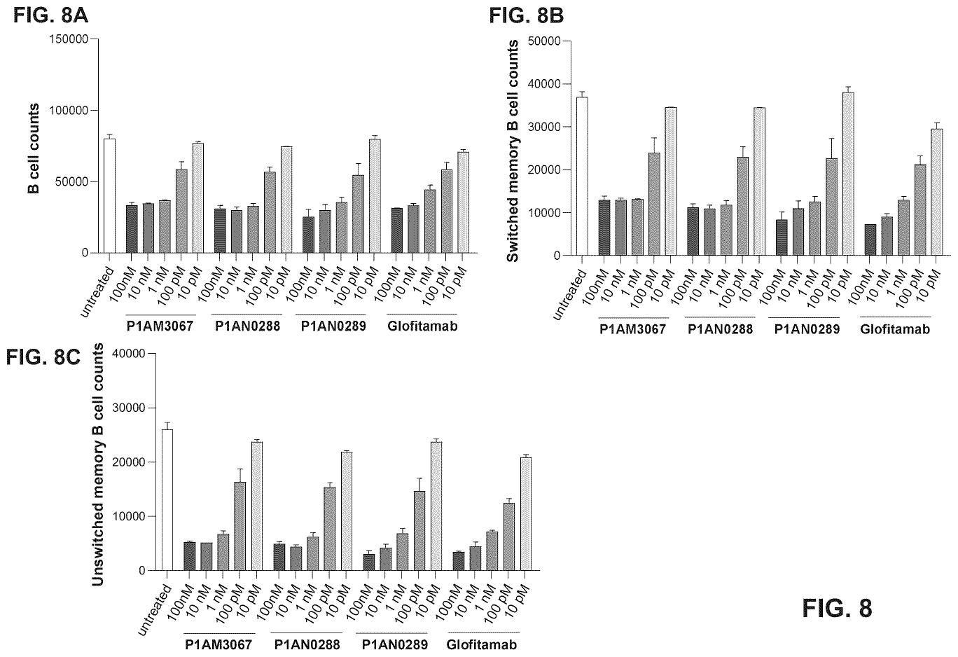

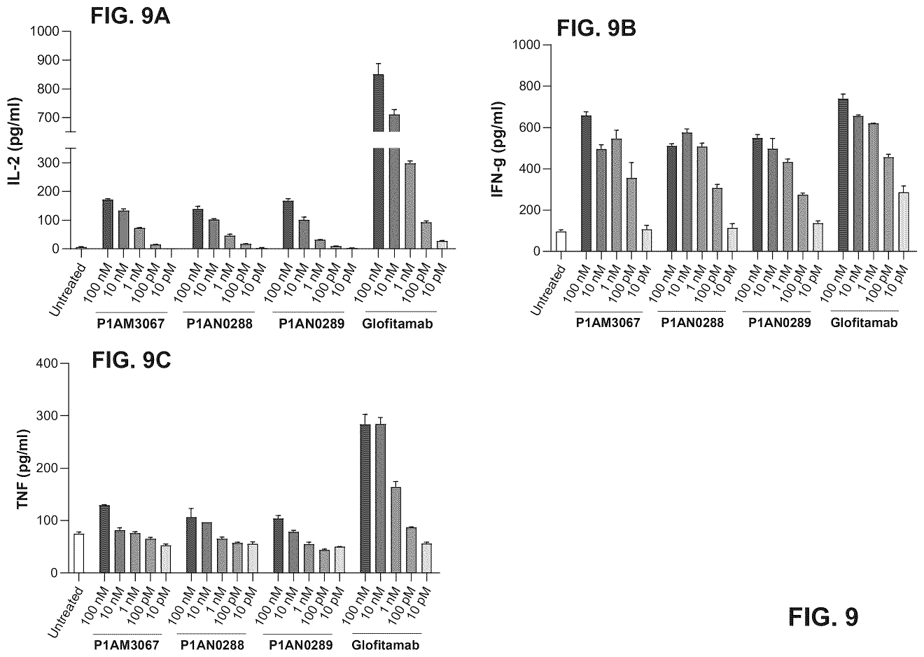

Potent B-cell depletion was reached with concentrations of >1 nM for all of the CD3×CD20 TCBs including glofitamab. Overall B-cell depletion and depletion of memory B-cell subsets was comparable between all TCBs and glofitamab (FIG. 8). In contrast, the TCBs with lower CD3 affinities showed strongly reduced IL-2 and TNF levels as well as decreased IFN-γ levels at 24 hours post-treatment, compared to corresponding doses of glofitamab (FIG. 9). Taken together, while the CD3×CD20 TCB molecules with lower CD3 affinity maintained the same potency in B-cell killing as glofitamab, they showed markedly reduced cytokine release.

### Example 11—Cytokine Release, Body Weight, and B-Cell Depletion Properties of Optimized Anti-CD3 (Multispecific) Antibodies In Vivo

The optimized anti-CD3 antibodies, as CD3×CD20 TCBs, were evaluated for their B-cell depletion potency, serum cytokine release and body weight kinetics in fully humanized BRGS-CD47 mice.

BRGS-CD47 female mice (immunodeficient mice expressing human SIRPA and CD47, developed by Roche and bred by Jackson Laboratories, Sacramento, CA) were humanized at 3-4 weeks of age by total body irradiation with 200 cGy, followed by intravenous injection of 10′ human hematopoietic stem cells. Twelve to fifteen weeks after stem cell injection, humanized BRGS-CD47 mice were screened for human cell engraftment in the blood by flow cytometry. The humanization and screening process was conducted at Jackson Laboratory, and only mice with a humanization rate greater than 20% (i.e., more than 20% of circulating human immune cells within all leukocytes) were shipped to Roche Innovation Center Zurich for subsequent experiments. Upon arrival, mice were maintained under specific-pathogen-free conditions and a 12 hour light/dark cycle according to committed guidelines (GV-Solas, Felasa, TierschG). The experimental study protocol was reviewed and approved by the local government (License ZH180-20). Health monitoring was performed regularly.

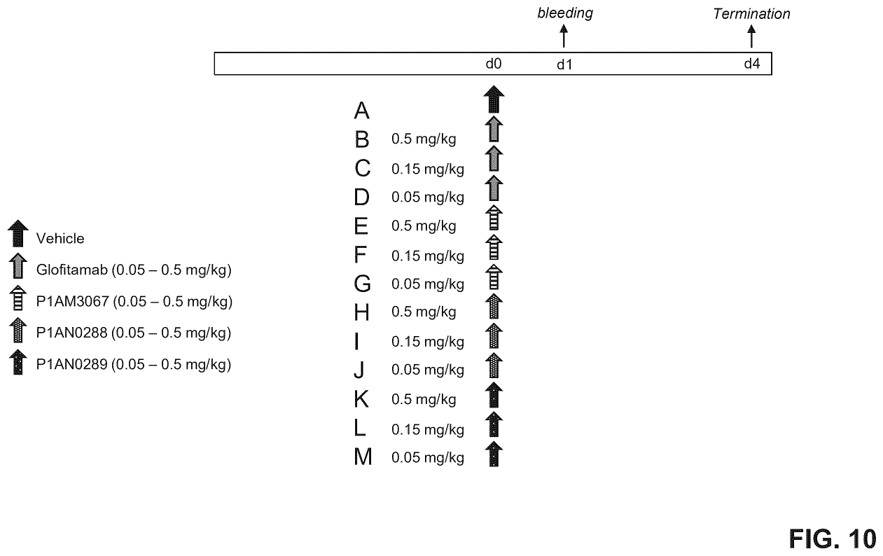

Mice were randomized into different treatment arms and treated with the various CD3×CD20 TCBs at three different doses each, or with histidine buffer (Vehicle; Group A) on day 0. FIG. 10 and Table 12 provide details on the doses, administration schedules, and routes of administration for all TCB molecules. All mice received the appropriate solutions. To achieve the correct amount of compounds per injection volume, the stock solutions were diluted with histidine buffer when necessary. Mice were bled, and serum was prepared for cytokine analysis on day 1 (24 hours after a single injection in all groups). Cytokine measurement was performed using the Luminex™ FLEXMAP 3D™ system with the Bio-Plex Pro Human Cytokine Assay, according to the manufacturer's instructions.

The study was terminated on day 4 post-injection, and blood and spleen samples were harvested from all mice and prepared for single-cell suspensions to evaluate B-cell counts by flow cytometry.

TABLE 12
Overview of study groups

| | No. of | | Dose | Injection | No. of |
|---|---|---|---|---|---|
| Group | animals | Compound | (mg/kg) | route | treatments |
| A | 5 | Vehicle | — | i.v. | once |
| B | 5 | Glofitamab | 0.5 | i.v. | once |
| C | 5 | Glofitamab | 0.15 | i.v. | once |
| D | 5 | Glofitamab | 0.05 | i.v. | once |
| E | 5 | P1AM3067 | 0.5 | i.v. | once |
| F | 5 | P1AM3067 | 0.15 | i.v. | once |
| G | 5 | P1AM3067 | 0.05 | i.v. | once |
| H | 5 | P1AN0288 | 0.5 | i.v. | once |
| I | 5 | P1AN0288 | 0.15 | i.v. | once |
| J | 5 | P1AN0288 | 0.05 | i.v. | once |
| K | 5 | P1AN0289 | 0.5 | i.v. | once |
| L | 5 | P1AN0289 | 0.15 | i.v. | once |
| M | 5 | P1AN0289 | 0.05 | i.v. | once |

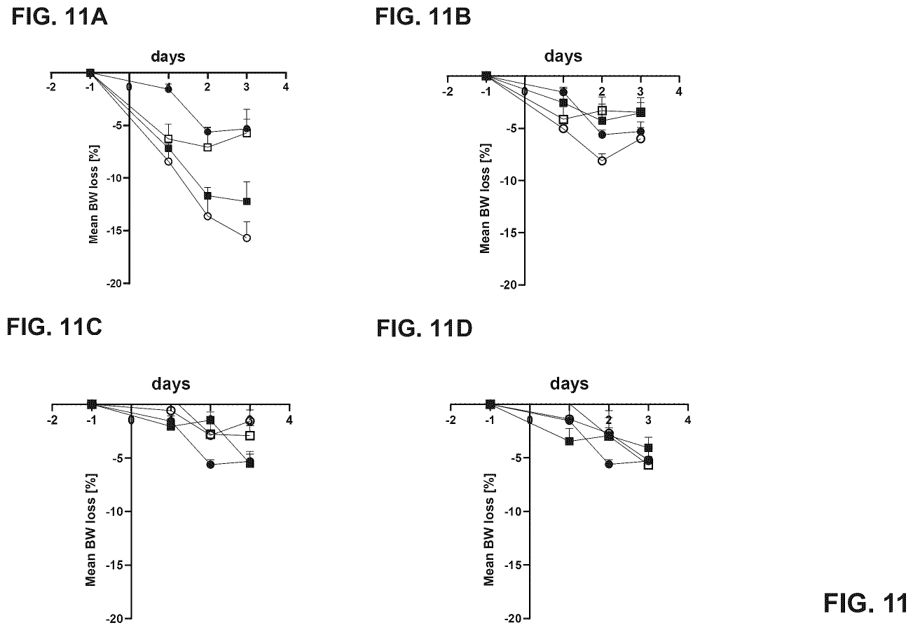

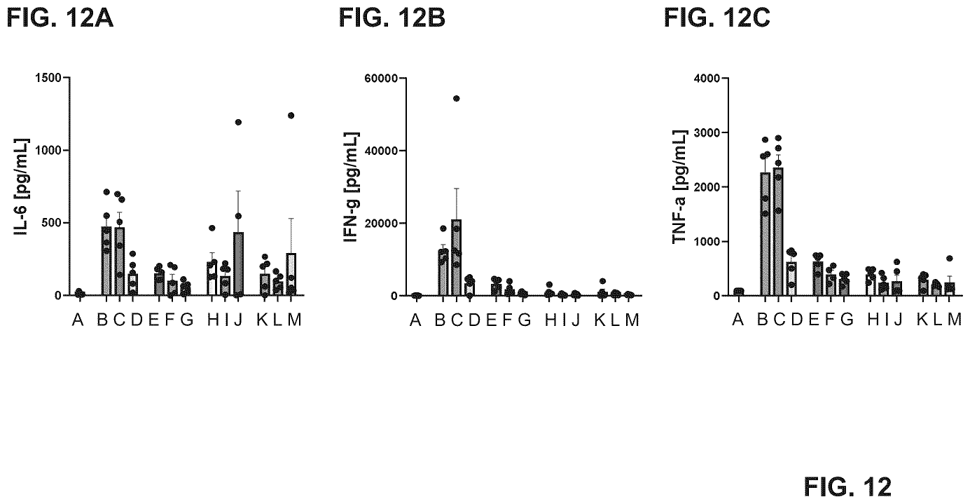

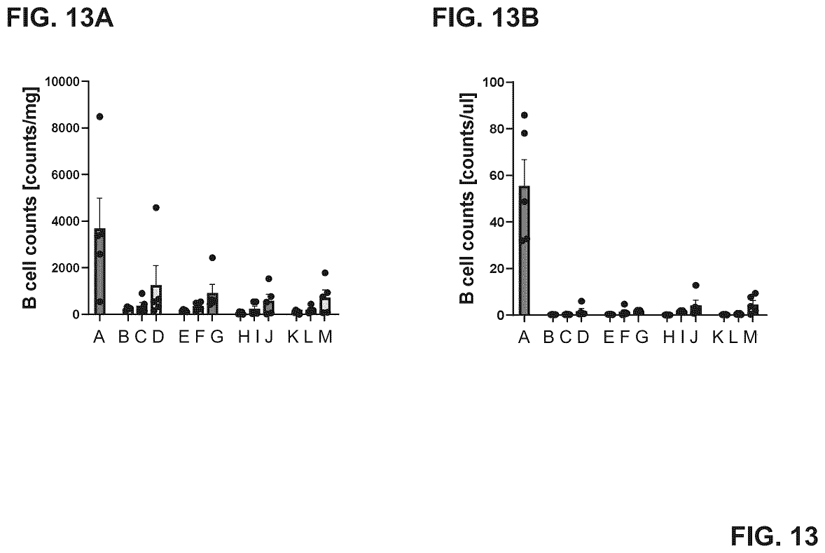

FIG. 11 illustrates the body weight kinetics in all groups as mean±SEM. The data indicate that mainly the higher doses of glofitamab resulted in significant body weight changes (0.5 and 0.15 mg/kg). The optimized molecules with lower CD3 affinities did not induce any measurable body weight drop. As depicted in FIG. 12, the TCBs with lower CD3 affinities show markedly reduced IL-6, IFN-γ, and TNF-α levels at 24 hours post-injection, as measured in serum samples, compared to corresponding doses of glofitamab. Importantly, the B-cell depletion properties in the spleen and periphery at study termination showed very comparable B-cell depletion to glofitamab (FIG. 13).

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. An antibody that binds to CD3, wherein the antibody comprises a heavy chain variable region (VH_{CD3}) and a light chain variable region (VL_{CD3}), wherein
a. the VH_{CD3} comprises the heavy chain complementarity determining region (HCDR) 1 of SEQ ID NO: 1, the HCDR 2 of SEQ ID NO: 9, and the HCDR 3 of SEQ ID NO: 10, and the VL_{CD3} comprises the light chain complementarity determining region (LCDR) 1 of SEQ ID NO: 5, the LCDR 2 of SEQ ID NO: 6, and the LCDR 3 of SEQ ID NO: 7;
b. the VH_{CD3} comprises the heavy chain complementarity determining region (HCDR) 1 of SEQ ID NO: 1, the HCDR 2 of SEQ ID NO: 2, and the HCDR 3 of SEQ ID NO: 3, and the VL_{CD3} comprises the light chain complementarity determining region (LCDR) 1 of SEQ ID NO: 5, the LCDR 2 of SEQ ID NO: 6, and the LCDR 3 of SEQ ID NO: 7; or
c. the VH_{CD3} comprises the heavy chain complementarity determining region (HCDR) 1 of SEQ ID NO: 1, the HCDR 2 of SEQ ID NO: 2, and the HCDR 3 of SEQ ID NO: 10, and the VL_{CD3} comprises the light chain complementarity determining region (LCDR) 1 of SEQ ID NO: 5, the LCDR 2 of SEQ ID NO: 6, and the LCDR 3 of SEQ ID NO: 13.

2. The antibody of claim 1, wherein
a. the VH_{CD3} comprises an amino acid sequence having at least 95%, at least 96%, at least 97%, at least 98%, at least 99% or 100% sequence identity to the VH sequence of SEQ ID NO: 11, and the VL_{CD3} comprises an amino acid sequence having at least 95%, at least 96%, at least 97%, at least 98%, at least 99% or 100% sequence identity to the VH sequence of SEQ ID NO: 8;
b. the VH_{CD3} comprises an amino acid sequence having at least 95%, at least 96%, at least 97%, at least 98%, at least 99% or 100% sequence identity to the VH sequence of SEQ ID NO: 4, and the VL_{CD3} comprises an amino acid sequence having at least 95%, at least 96%, at least 97%, at least 98%, at least 99% or 100% sequence identity to the VH sequence of SEQ ID NO: 8; or
c. the VH_{CD3} comprises an amino acid sequence having at least 95%, at least 96%, at least 97%, at least 98%, at least 99% or 100% sequence identity to the VH sequence of SEQ ID NO: 12, and the VL_{CD3} comprises an amino acid sequence having at least 95%, at least 96%, at least 97%, at least 98%, at least 99% or 100% sequence identity to the VH sequence of SEQ ID NO: 14.

3. An antibody that binds to CD3, comprising
a. a heavy chain variable region (VH_{CD3}) comprising the amino acid sequence of SEQ ID NO: 11, and a light chain variable region (VL_{CD3}) comprising the amino acid sequence of SEQ ID NO: 8;
b. a heavy chain variable region (VH_{CD3}) comprising the amino acid sequence of SEQ ID NO: 4, and a light chain variable region (VL_{CD3}) comprising the amino acid sequence of SEQ ID NO: 8; or
c. a heavy chain variable region (VH_{CD3}) comprising the amino acid sequence of SEQ ID NO: 12, and a light chain variable region (VL_{CD3}) comprising the amino acid sequence of SEQ ID NO: 14.

4. The antibody of any one of claims 1-3, wherein the antibody is
a. a full-length antibody, particularly a full-length IgG antibody; or
b. an antibody fragment, particularly an antibody fragment selected from the group of an Fv molecule, a scFv molecule, a Fab molecule, and a F(ab′)₂ molecule.

5. The antibody of any one of claims 1-4, wherein the antibody is a multispecific antibody, particularly a bispecific antibody.

6. The antibody of claim 5, wherein the antibody comprises
a. a first antigen binding domain that binds to CD3, comprising the VH_{CD3} and the VL_{CD3}, and
b. a second and optionally a third antigen binding domain that bind to a second antigen.

7. The antibody of claim 6, wherein the first antigen binding domain, the second antigen binding domain and/or the third antigen binding domain is a Fab molecule.

8. The antibody of claim 6 or 7, wherein the first antigen binding domain is a Fab molecule wherein the variable domains VH and VL or the constant domains CH1 and CL of the Fab heavy and light chains are replaced by each other.

9. The antibody of any one of claims 6-8, wherein the second antigen binding domain and, where present, the third antigen binding domain are each a conventional Fab molecule.

10. The antibody of any one of claims 6-9, wherein in the constant domain CL of the second antigen binding domain and, where present, the third antigen binding domain the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the constant domain CH1 of the second antigen binding domain and, where present, the third antigen binding domain the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

11. The antibody of any one of claims 6-10, wherein the second antigen is a target cell antigen, particularly a B-cell antigen.

12. The antibody of any one of claims 6-11, wherein the second antigen is CD20.

13. The antibody of claim 12, wherein the second antigen binding domain and, where present, the third antigen binding domain comprises a heavy chain variable region (VH_{CD20}) comprising the heavy chain complementarity determining region (HCDR) 1 of SEQ ID NO: 15, the HCDR 2 of SEQ ID NO: 16, and the HCDR 3 of SEQ ID NO: 17, and a light chain variable region (VL_{CD20}) comprising the light chain complementarity determining region (LCDR) 1 of SEQ ID NO: 19, the LCDR 2 of SEQ ID NO: 20, and the LCDR 3 of SEQ ID NO: 21.

14. The antibody of claim 12 or 13, wherein the second antigen binding domain and, where present, the third antigen binding domain comprises a VH_{CD20} comprising an amino acid sequence having at least 95%, at least 96%, at least 97%, at least 98%, at least 99% or 100% sequence identity to the VH sequence of SEQ ID NO: 18, and a VL_{CD20} comprising an amino acid sequence having at least 95%, at least 96%, at least 97%, at least 98%, at least 99% or 100% sequence identity to the VH sequence of SEQ ID NO: 22.

15. The antibody of any one of claims 6-14, wherein the first antigen binding domain, the second antigen binding domain and, where present, the third antigen binding domain are each a Fab molecule and (i) the first antigen binding domain is fused at the C-terminus of the Fab heavy chain to the N-terminus of the Fab heavy chain of the second antigen binding domain, or (ii) the second antigen binding domain is fused at the C-terminus of the Fab heavy chain to the N-terminus of the Fab heavy chain of the first antigen binding domain.

16. The antibody of claim 15, wherein
a. the antibody comprises an Fc domain composed of a first and a second subunit,
b. (i) the first antigen binding domain is fused at the C-terminus of the Fab heavy chain to the N-terminus of one of the subunits of the Fc domain, or (ii) the second antigen binding domain is fused at the C-terminus of the Fab heavy chain to the N-terminus of one of the subunits of the Fc domain, and
c. where present, the third antigen binding domain is fused at the C-terminus of the Fab heavy chain to the N-terminus of the other one of the subunits of the Fc domain.

17. The antibody of any one of claims 1-16, wherein the antibody comprises an Fc domain composed of a first and a second subunit.

18. The antibody of claim 16 or 17, wherein the Fc domain is an IgG Fc domain, particularly an IgG₁ Fc domain, more particularly a human IgG₁ Fc domain.

19. The antibody of any one of claims 16-18, wherein the Fc domain comprises a modification, particularly an amino acid substitution or combination of amino acid substitutions, promoting the association of the first and the second subunit of the Fc domain.

20. The antibody of any one of claims 16-19, wherein the Fc domain comprises a modification, particularly an amino acid substitution or combination of amino acid substitutions, that reduces the binding affinity to an Fc receptor and/or effector function of the antibody, as compared to a corresponding antibody comprising an Fc domain without such modification.

21. The antibody of any one of claims 16-20, wherein the Fc domain comprises a modification, particularly an amino acid substitution or combination of amino acid substitutions, that increases the binding affinity to an FcRn receptor and/or the serum half-life of the antibody, as compared to a corresponding antibody comprising an Fc domain without such modification.

22. The antibody of any one of claims 6-21, wherein the antibody comprises
a. a first heavy chain (HC1) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 23, a first light chain (LC1) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 28, a second heavy chain (HC2) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 25, and a second light chain (LC2) (particularly two LC2) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 26;
b. a first heavy chain (HC1) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 23, a first light chain (LC1) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 27, a second heavy chain (HC2) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 25, and a second light chain (LC2) (particularly two LC2) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 26; or
c. a first heavy chain (HC1) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 24, a first light chain (LC1) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 29, a second heavy chain (HC2) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 25, and a second light chain (LC2) (particularly two LC2) comprising an amino acid sequence having at least 90%, at least 91%, at least 92%, at least 93%, at least 94%, at least 95%, at least 96%, at least 97%, at least 98%, at least 99% or 100% sequence identity to the amino acid sequence of SEQ ID NO: 26.

23. An isolated polynucleotide encoding the antibody of any one of claims 1-22.

24. A host cell comprising the isolated polynucleotide of claim 23.

25. A method of producing an antibody that binds to CD3, comprising the steps of (a) culturing the host cell of claim 24 under conditions suitable for the expression of the antibody and optionally (b) recovering the antibody.

26. An antibody that binds to CD3 produced by the method of claim 25.

27. A pharmaceutical composition comprising the antibody of any one of claims 1-22 or 26 and a pharmaceutically acceptable carrier.

28. The antibody of any one of claims 1-22 or 26 or the pharmaceutical composition of claim 27 for use as a medicament.

29. The antibody of any one of claims 1-22 or 26 or the pharmaceutical composition of claim 27 for use in the treatment of a disease.

30. The antibody or pharmaceutical composition for use of claim 29, wherein the disease is cancer or an autoimmune disease.

31. Use of the antibody of any one of claims 1-22 or 26 or the pharmaceutical composition of claim 27 in the manufacture of a medicament.

32. Use of the antibody of any one of claims 1-22 or 36 or the pharmaceutical composition of claim 27 in the manufacture of a medicament for the treatment of a disease.

33. The use of claim 32, wherein the disease is cancer or an autoimmune disease.

34. A method of treating a disease in an individual, comprising administering to said individual an effective amount of the antibody of any one of claims 1-22 or 26 or the pharmaceutical composition of claim 37.

35. The method of claim 34, wherein the disease is cancer or an autoimmune disease.

36. The invention as described hereinbefore.


SEQUENCE LISTING (44 sequences)

SEQ ID NO: 1 - protein, 5 aa, synthetic construct
SYAMN

SEQ ID NO: 2 - protein, 19 aa, synthetic construct
RQRTQYNNREDYYADSVKG

SEQ ID NO: 3 - protein, 14 aa, synthetic construct
HSNFPSSYDSYFQY

SEQ ID NO: 4 - protein, 125 aa, synthetic construct
EVQLLESGGGLVQPGGSLRLSCAASGFTFSSYAMNWVRQAPGKGLEWVGRQRTQYNNREDYYADSVKGRFTISRDDSKNTLYLQMNSLRAEDTAVYYCVRHSNFPSSYDSYFQYWGQGTLVTVSS

SEQ ID NO: 5 - protein, 14 aa, synthetic construct
GSSTGAVTTSNYAN

SEQ ID NO: 6 - protein, 7 aa, synthetic construct
GTNKLAP

SEQ ID NO: 7 - protein, 9 aa, synthetic construct
ALWYSNLWY

SEQ ID NO: 8 - protein, 109 aa, synthetic construct
QAVVTQEPSLTVSPGGTVTLTCGSSTGAVTTSNYANWVQEKPGQPPRGLIGGTNKLAPGTPARFSGSLLGGKAALTLSGAQPEDEAEYYCALWYSNLWYFGGGTKLTVL

SEQ ID NO: 9 - protein, 19 aa, synthetic construct
RQRTQYHDRADYYADSVKG

SEQ ID NO: 10 - protein, 14 aa, synthetic construct
HSNFPTSYDSYFEY

SEQ ID NO: 11 - protein, 125 aa, synthetic construct
EVQLLESGGGLVQPGGSLRLSCAASGFTFSSYAMNWVRQAPGKGLEWVGRQRTQYHDRADYYADSVKGRFTISRDDSKNTLYLQMNSLRAEDTAVYYCVRHSNFPTSYDSYFEYWGQGTLVTVSS

SEQ ID NO: 12 - protein, 125 aa, synthetic construct
EVQLLESGGGLVQPGGSLRLSCAASGFTFSSYAMNWVRQAPGKGLEWVGRQRTQYNNREDYYADSVKGRFTISRDDSKNTLYLQMNSLRAEDTAVYYCVRHSNFPTSYDSYFEYWGQGTLVTVSS

SEQ ID NO: 13 - protein, 9 aa, synthetic construct
ALWYSNLWV

SEQ ID NO: 14 - protein, 109 aa, synthetic construct
QAVVTQEPSLTVSPGGTVTLTCGSSTGAVTTSNYANWVQEKPGQPPRGLIGGTNKLAPGTPARFSGSLLGGKAALTLSGAQPEDEAEYYCALWYSNLWVFGGGTKLTVL

SEQ ID NO: 15 - protein, 5 aa, synthetic construct
YSWIN

SEQ ID NO: 16 - protein, 17 aa, synthetic construct
RIFPGDGDTDYNGKFKG

SEQ ID NO: 17 - protein, 10 aa, synthetic construct
NVFDGYWLVY

SEQ ID NO: 18 - protein, 119 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 19 - protein, 16 aa, synthetic construct
RSSKSLLHSNGITYLY

SEQ ID NO: 20 - protein, 7 aa, synthetic construct
QMSNLVS

SEQ ID NO: 21 - protein, 9 aa, synthetic construct
AQNLELPYT

SEQ ID NO: 22 - protein, 112 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 23 - protein, 671 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCGGGGSGGGGQAVVTQEPSLTVSPGGTVTLTCGSSTGAVTTSNYANWVQEKPGQPPRGLIGGTNKLAPGTPARFSGSLLGGKAALTLSGAQPEDEAEYYCALWYSNLWYFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 671 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCGGGGSGGGGQAVVTQEPSLTVSPGGTVTLTCGSSTGAVTTSNYANWVQEKPGQPPRGLIGGTNKLAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 448 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 26 - protein, 219 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 232 aa, synthetic construct
EVQLLESGGGLVQPGGSLRLSCAASGFTFSSYAMNWVRQAPGKGLEWVGRQRTQYNNREDYYADSVKGRFTISRDDSKNTLYLQMNSLRAEDTAVYYCVRHSNFPSSYDSYFQYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 232 aa, synthetic construct
EVQLLESGGGLVQPGGSLRLSCAASGFTFSSYAMNWVRQAPGKGLEWVGRQRTQYHDRADYYADSVKGRFTISRDDSKNTLYLQMNSLRAEDTAVYYCVRHSNFPTSYDSYFEYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 232 aa, synthetic construct
EVQLLESGGGLVQPGGSLRLSCAASGFTFSSYAMNWVRQAPGKGLEWVGRQRTQYNNREDYYADSVKGRFTISRDDSKNTLYLQMNSLRAEDTAVYYCVRHSNFPTSYDSYFEYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 186 aa, Homo sapiens
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 31 - protein, 177 aa, Macaca fascicularis
QDGNEEMGSITQTPYQVSISGTTVILTCSQHLGSEAQWQHNGKNKEDSGDRLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

SEQ ID NO: 32 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 33 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 34 - protein, 107 aa, Homo sapiens [sequence omitted]

SEQ ID NO: 35 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 36 - protein, 328 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 37 - protein, 9 aa, synthetic construct
GGGGSGGGG

SEQ ID NO: 38 - protein, 360 aa, synthetic construct [sequence omitted]

SEQ ID NO: 39 - protein, 325 aa, synthetic construct
FKIPIEELEDRVFVNCNTSITWVEGTVGTLLSDITRLDLGKRILDPRGIYRCNGTDIYKDKESTVQVHYRMCRSEQLYFQGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKSGGLNDIFEAQKIEWHE

SEQ ID NO: 40 - protein, 19 aa, synthetic construct
RIRSKYNNYATYYADSVKG

SEQ ID NO: 41 - protein, 14 aa, synthetic construct
ASNFPASYVSYFAY

SEQ ID NO: 42 - protein, 125 aa, synthetic construct
EVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSS

SEQ ID NO: 43 - protein, 7 aa, synthetic construct
GTNKRAP

SEQ ID NO: 44 - protein, 109 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL